(19) United States
(12) Patent Application Publication
(10) Pub. No.: US 2012/0271036 A1
(43) Pub. Date: Oct. 25, 2012

(54) Solution Assay and High Through-Put Screen to Probe Interaction Between Human Cullin-Ring Ligase Complex and HIV-VIF Protein

(75) Inventors: Harold C. Smith, Rochester, NY (US); Jason D. Salter, Rochester, NY (US); Joseph E. Wedekind, Rochester, NY (US)

(73) Assignee: UNIVERSITY OF ROCHESTER

(21) Appl. No.: 13/504,690

(22) PCT Filed: Oct. 29, 2010
(86) PCT No.: PCT/US10/54657

§ 371 Date: Jul. 16, 2012

Related U.S. Application Data
(60) Provisional application No. 61/256,166, filed on Oct. 29, 2009.

(51) Int. Cl. C07K 14/00; C07K 19/00; C12N 15/62; C12N 5/071; C12N 15/63; C12P 21/00; G01N 33/566

(52) U.S. Cl. 530/350; 435/69.1; 435/69.7; 436/501; 435/375; 435/320.1; 536/23.4

(57) ABSTRACT

The present invention relates to large-scale production of ElonginB, ElonginC, Vif, and Cullin5. The present invention provides an assay for screening any agent that inhibits the ability of Vif to bind with Cul5. The invention provides an agent identified by the screening methods and methods of treatment using the identified agent.

### BACKGROUND OF THE INVENTION

Under normal circumstances, HIV-1 infection leads to the production of the viral protein Vif (viral infectivity factor). This viral protein is essential to evade the host's own APOBEC3G (A3G) and A3F defense factors. A3G is a member of the APOBEC3 (Apolipoprotein B mRNA-editing enzyme catalytic polypeptide-like editing complex 3) family of cytidine deaminases that deaminate deoxycytidine (dC) to deoxyuridine (dU) in a sequence specific manner on single stranded DNA and have potent antiretroviral activity (Cullen, 2006, J Virol 80:1067-76). In humans there are eight A3 genes (hA3A-H) clustered at one locus of chromosome 22 (Jarmuz, et al., 2002, Genomics 79:285-96). The A3 family of proteins is a subset of the APOBEC-related protein family, which also includes APOBEC-1 and AID (activation induced deaminase). APOBEC-1 is the catalytic subunit of a complex expressed in gastrointestinal tissue that deaminates a specific C to U on apolipoprotein B (apoB) mRNA thereby creating a premature stop codon, altering the size and biological function of the expressed apoB protein (Wedekind, et al., 2003, Trends Genet. 19:207-16). AID catalyzes target deamination of dC to dU on ssDNA with the variable region and class swithe region of immunoglobulin genes upon activation of B cells (Bransteitter, et al., 2003, Proc Natl Acad Sci USA 100:4102-7).

APOBEC proteins possess a zinc-dependent deaminase (ZDD) motif consensus sequence (H-x-E-X₂₇₋₂₈-P-C-xx-C) (Jarmuz et al., 2002, Genomics 79:285-96). This ZDD motif is also found in nucleotide cytidine deaminases and x-ray crystallographic models of nucleotide cytidine deaminases are available from E. coli (Betts et al., 1994, J Mol Biol 235:635-56), B. subtillis (Johansson et al., 2002, Biochemistry 41:2563-70), S cerevisiae (Xie et al., 2004, Proc Natl Acad Sci USA 101:8114-9), and human (Chung et al., 2005, J Med Chem 48:658-60). Each has a β₁β₂α₁β₃α₂β₄β₅ core catalytic cytidine deaminase fold (CDA) (Mian et al., 1998, J Comput Biol 5:57-72) and sequence alignments suggest that APOBEC proteins share this core catalytic fold (Wedekind et al., 2003, Trends Genet. 19:207-16); however, it is unclear how APOBEC proteins bind their nucleic acid substrates. Furthermore, A3G possesses two ZDD motifs in tandem, presumably arising through gene duplication, and thus is predicted to have two CDAs.

A3G is expressed in blood lymphocytes (Jarmuz et al., 2002, Genomics 79:285-96; Bishop et al., 2004, Curr Biol 14:1392-6); in CD4+ T cells, the primary target for HIV-1, hA3G is present as either a high or a low molecular mass form, HMM and LMM respectively, depending on cellular conditions and location (Chiu et al., 2005, Nature 435:108-14; Chelico et al., 2006, Nat Struct Mol Biol 13:392-9).

The LMM form of A3G is a potent post-entry restriction factor that protects resting CD4⁺ T cells and monocytes in peripheral blood from HIV-1 infection. In unstimulated CD4⁺ T cells in peripheral blood, A3G is predominantly present as a dimer (LMM) (Chiu et al., 2005, Nature 435:108-14), and blocks viral replication by two distinct mechanisms. First, LMM A3G induces dC to dU mutations on viral reverse transcripts which are transiently single stranded after RNase H dependent degradation of the viral RNA template (Chelico et al., 2006, Nat Struct Mol Biol 13:392-9; Lecossier et al., 2003, Science 300:1112; Mangeat et al., 2003, Nature 424:99-103; Zhang et al., 2003, Nature 424:94-8). This dU-laden DNA induces dG to dA mutations on the positive proviral DNA causing abortive infection. Secondly, evidence exists for an alternative, non-enzymatic, mechanism for blocking HIV-1 replication that involves binding of hA3 G to RNA of the reverse transcriptase complex thereby delaying the appearance of viral reverse transcripts. The N-terminal ZDD motif of A3G is not catalytically active and in vitro, it has been shown to bind RNA non-specifically (Newman et al., 2005, Curr Biol 15:166-70). Taken together with the fact that A3G mutants lacking a functional C-terminal ZDD still display an anti-retroviral phenotype (Newman et al., 2005, Curr Biol 15:166-70) this highly suggests that both ZDDs of A3G contribute to the antiretroviral activity by different mechanisms. The non-enzymatic mechanism has also been implicated in hA3G inhibition of hepatitis B (Turelli et al., 2004, Science 303:1829), human T-cell leukemia viral infections (Sasada et al., 2005, Retrovirology 2:32; Strebel, 2005, Retrovirology 2:37) and the non-autonomous retroelements Alu and hY (Chiu et al., 2006, Proc Natl Acad Sci USA 103:15588-93).

The HMM form of A3G does not restrict HIV-1. When naïve CD4⁺ T cells locate to lymphoid tissue or when they are stimulated with mitogens, A3G is sequestered into HMM (5-15 MDa) ribonucleoprotein complexes (Chiu et al., 2005, Nature 435:108-14; Kreisberg et al., 2006, J Exp Med 203:865-70). The HMM forms of A3G are inactive against HIV-1; however, RNase treatment of isolated HMM complexes restores the activity and the LMM A3G form (Chiu et al., 2005, Nature 435:108-14; Chelico et al., 2006, Nat Struct Mol Biol 13:392-9). Thus, CD4⁺ T cells in lymphoid tissue and stimulated CD4⁺ T cells of peripheral blood are susceptible to HIV-1 infection; but, during an HIV-1 (Δvif) infection, A3G is packaged into budding virions (Marin et al., 2003, Nat Med 9:1398-403; Mehle et al., 2004, Genes Dev 18:2861-6; Mariani et al., 2003, Cell 114:21-31; Rose et al., 2004, Trends Mol Med 10:291-7). In subsequently infected cells, encapsidated A3G associates with the reverse transcriptase complex and is a potent post-entry restriction factor, thereby preventing a sustainable HIV-1 infection (Mangeat et al., 2003, Nature 424:99-103).

HIV-1 vif is necessary to overcome the antiretroviral effects of hA3G. In HIV-1 wild type infections, vif prevents the encapsidation of A3G into virions. Vif, common to nearly all lentiviruses (Oberste et al., 1992, Virus Genes 6:95-102), is a 23 kD accessory protein localized to the cytoplasm and expressed late in the viral life cycle (Mehle et al., 2004, Genes Dev 18:2861-6; Yang et al., 1996, J Biol Chem 271:10121-9). After infecting a susceptible cell, HIV-1 vif specifically targets A3G for polyubiquitination and degradation by the 23S proteasome (Marin et al., 2003, Nat Med 9:1398-403; Yu et al., 2003, Science 302:1056-60; Sheehy et al., 2003, Nat Med 9:1404-7; Mehle et al., 2004, J Biol Chem 279:7792-8; Stopak et al., 2003, Mol Cell 12:591-601). Protein ubiquitination is complex, involving the coordinated activities of an E1 ubiquitin activating enzyme, an E2 ubiquitin conjugating enzyme, and an E3 ubiquitin ligase complex which facilitates transfer of an activated ubiquitin from E2 to a specific substrate. Vif acts as the substrate receptor specifically recruiting A3G to a cullin5 (Cul5) dependent E3 ligase comprising the substrate recognition adaptor molecules, Elongin B and Elongin C (EloB/C), and the E2 binding platform RING protein, Rbx2 (Shirakawa et al., 2006, Virology 344:263-6; Kobayashi, Met al., 2005, J Biol Chem 280:18573-8).

Vif binds EloB/C through a specific BC-box consensus sequence ¹⁴⁴(S,T,A,P)LxxxCxxx(L,I,A,V)¹⁵³, similar to BC-boxes of cellular suppressor of cytokine signaling (SOCS) proteins (Yu et al., 2004, Genes Dev 18:2867-72). The BC box of vif is predicted to form part of an alpha helix and bind tightly to a hydrophobic pocket of EloC (Yu et al., 2004, Genes Dev 18:2867-72) in a manner analogous to that for cellular BC-box proteins, as exemplified by the von Hippel-Lindau (VHL) protein. VHL recruits HIF-1α (hypoxia inducible factor) to an E3 ligase complex for polyubiquitination, binding EloB/C with its BC-box. The crystal structure of this ternary complex has been solved (Stebbins et al., 1999, Science 284:455-61; Hon et al., 2002, Nature 417:975-8). In vivo and in vitro studies in which one or more of the residues in the vif BC-box have been mutated indicate this motif is required for vif-mediated polyubiquitination of A3G and for successful HIV infection; not surprisingly, this is the most conserved region of lentiviral vif (Yu et al., 2004, Genes Dev 18:2867-72).

Vif specifically binds Cul5 through conserved hydrophobic residues within a novel Zn²⁺ binding ¹⁰⁸H-x₅-C-x₁₇₋₁₈-C-x₃₋₅-H¹³⁹ (HCCH) motif that spans 31 residues and is located just N-terminal of the BC-box (Xiao et al., 2006, Virology 349:290-9; Mehle et al., 2006, J Biol Chem 281:17259-65; Luo et al., 2005, Proc Natl Acad Sci USA 102:11444-9). A conserved hydrophobic patch (consensus sequence F-X₄-Φ-X₂-A-Φ) located between the two cysteines of the HCCH motif are predicted to be on the same face of an alpha helix that interacts with an exposed loop sequence that is unique to Cul5, among the Cullin family (Xiao et al., 2006, Virology 349:290-9). Interestingly, this mode of binding Cul5 differs from that of cellular substrate receptors that bind to Cul5 through a conserved sequence, called the Cul-5 box, that is C-terminal of the BC-box (Kamura et al., 2004, Genes Dev 18:3055-65).

Numerous N-terminal amino acids of vif have been shown to be involved in A3G and A3F binding. The N-terminus of vif has an unusually enriched tryptophan content, all of which are highly conserved. In particular, mutation of either Trp5, 21, 38, or 89 significantly reduces HIV-1 infectivity (Tian et al., 2006, J Virol 80:3112-5). Recruitment of A3G to the E3 ubiquitin ligase complex is thought to occur through an interaction between the N-terminus of vif and the N-terminal ZDD domain of A3G. This interaction has been shown to be disrupted by a single mutation of the negatively charged Asp 128 of A3G to a positively charged residue (Bogerd et al., 2004, Proc Natl Acad Sci USA 101:3770-4). High resolution structural models should reveal the chemical basis for binding of vif and A3G complexes. However, the biochemistry of vif cannot be adequately studied because recombinant vif is inherently insoluble.

Vif is a notoriously difficult protein to work with due to its tendency to aggregate during purification. Small fragments of Vif alone are not expressed well in E. coli nor are they soluble. Given these problems with Vif expression, high throughput assays to identify antiviral compounds that prevent Vif-dependent degradation of APOBEC3G/APOBEC3F have not been possible. Similarly the problems with Vif expression and solubility have been a roadblock to developing high-throughput assays for the discovery of compounds that inhibit Vif interactions with the ubiquitination machinery. There is a need in the art for methods of producing sufficient amounts of soluble vif in order to explore the biochemistry of vif. The present invention satisfies this need as well as other needs regarding treatment of HIV infection.

### SUMMARY OF THE INVENTION

The invention provides a method of producing soluble Vif. The method comprises contacting a cell with an isolated nucleic acid sequence encoding ElonginB, Vif, and ElonginC in a cell; expressing ElonginB, Vif, and ElonginC polypeptide from the cell; and isolating ElonginB, Vif, and ElonginC polypeptide from the cell, wherein the Vif so isolated is soluble.

In one embodiment, ElonginB and Vif are expressed as a fusion polypeptide having a protease cleavage site therebetween, and ElonginC is expressed as an additional polypeptide.

In another embodiment, the fusion polypeptide comprises amino acids 1-98 of ElonginB and amino acids 102-173 of Vif, and the additional polypeptide comprises amino acids 17-112 of ElonginC.

The invention provides a method of producing soluble Cullin5. The method comprises contacting a cell with an isolated nucleic acid sequence encoding Cullin5 having point mutations V341R and L345D; expressing the Cullin5 polypeptide from the cell; and isolating the Cullin5 from the cell, wherein the Cullin5 so isolated is soluble.

The invention provides an isolated protein complex comprising Vif, ElonginB, and ElonginC, wherein Vif is able to bind Cullin5.

In one embodiment, the isolated protein complex of further comprising Cullin5.

In another embodiment, the isolated protein complex comprises amino acids 1-98 of ElonginB fused to a protease cleavage site which in turn is fused to amino acids 102-173 of Vif, and ElonginC comprises amino acids 17-112.

In another embodiment, the isolated protein complex comprises amino acids 1-104 of ElonginB fused to a protease cleavage site which in turn is fused to amino acids 102-173 of Vif, and ElonginC comprises amino acids 17-112.

In another embodiment, the isolated protein complex comprises amino acids 1-118 of ElonginB fused to a protease cleavage site which in turn is fused to amino acids 102-173 of Vif, and ElonginC comprises amino acids 17-112.

In another embodiment, the isolated protein complex comprises amino acids 1-118 of ElonginB fused to a protease cleavage site which in turn is fused to amino acids 95-173 of Vif, and ElonginC comprises amino acids 17-112.

The invention provides an isolated Cullin5 polypeptide comprising point mutations V341 and L345D.

The invention provides a method of identifying a compound that binds to Vif. The method comprises contacting Vif with a test compound under conditions that are effective for binding of the compound with Vif; and detecting whether or not the test compound binds to Vif, wherein detection of the test compound bound to Vif identifies a compound that binds to Vif.

The invention provides a method of identifying a compound that inhibits the interaction between Vif and Cullin5. The method comprises contacting a protein complex comprising ElonginB, ElonginC, Vif, and Cullin5 with a test compound under conditions that are effective for binding of Vif to Cullin5; and detecting whether or not the test compound disrupts binding of Vif to Cullin5, wherein when binding is disrupted, the test compound has inhibited the interaction between Vif and Cullin5.

In one embodiment, the test compound that disrupts the binding between Vif and Cullin5 is an inhibitor of lentiviral infectivity.

In another embodiment, the method is a high throughput method.

In another embodiment, the high throughput method is Förster quenched resonance energy transfer (FqRET).

The invention includes compounds identified by the screening methods discussed elsewhere herein.

The invention provides a method for inhibiting infectivity of a lentivirus. The method comprising contacting a cell which is producing the virus with an antiviral-effective amount of a compound identified by the method of the invention, wherein the antiviral-effective amount of the compound does not substantially affect proteins in the cell other than lentivirus Vif. Preferably, the lentivirus expresses Vif. More preferably, the lentivirus is HIV.

In one embodiment, the compound inhibits the interaction of Vif with cellular Cullin5-E3 ubiquitin ligase, thereby preventing the degradation of the viral inhibitor, Apobec3G, and thus allowing the Apobec3G to inhibit viral infectivity.

The invention provides a method for inhibiting Vif protein activity in a cell. The method comprises contacting Vif protein with an inhibitory-effective amount of a compound identified by the methods of the invention, wherein the inhibitory-effective amount of compound does not substantially affect proteins in the cell other than Vif.

The invention provides a vector for coexpression of at least two target polynucleotides, wherein the first target polynucleotide comprises sequences encoding amino acids 1-98 of ElonginB and amino acids 102-173 of Vif having a flexible linker therebetween, and the second target polynucleotide comprises sequences encoding ElonginC, further wherein the linker comprises sequences encoding a protease cleavage site.

The invention provides an isolated nucleic acid molecule comprising sequences encoding amino acids 1-98 of ElonginB and amino acids 102-173 of Vif having a flexible linker sequence therebetween, wherein the linker sequence comprises sequences encoding a protease cleavage site.

The invention provides an isolated nucleic acid molecule comprising sequences encoding Cullin5, wherein the encoded Cullin5 comprises the V341 and L345D point mutations.

This invention provides compositions and methods for detecting the interaction between viral infectivity factor (Vif) and Cullin 5 (Cul5). The invention includes methods for identifying compounds that effect the binding between Vif and Cul5. The identified compounds are useful for treating a virus infection associated with Vif. Preferably, the virus infection associated with Vif is HIV.

The present invention is based on the discovery that large amounts of soluble Vif can be produced when Vif is fused to the C-terminus of Elongin B (e.g., Vif/Elogin B fusion protein). Preferably, the Vif/Elogin B fusion protein is expressed from a signal vector. In some instances, the Vif/Elogin B fusion protein is co-expressed with Elongin C from a single vector. The resulting two-polypeptide complex (Vif/Elogin B and Elogin C) can further be processed by designed proteolysis sites to generate a folded tripartite complex comprising ElonginB/ElonginC and Vif.

The present invention is based on the discovery that the Vif-interacting domain of Cullin5 (Cul5) can be produced recombinantly. Cul5 can be engineered to be fused with a reporter tag so that the tag can be removed when needed. Any tag can be used in the context of the invention. As a non-limiting example, Cul5 can be produced in the context of a fusion protein comprising glutathione-S-transferase (GST). In order to produce the soluble Cullin5 interacting domains, site directed mutants were introduced to the gene. Preferably, the two mutations introduced to Cullin5 to increase solubility are V341R and L345D.

Accordingly, the invention provides an assay for determining the binding between Cul5 with Vif, wherein Vif is in the context of the ElonginB/ElonginC/Vif complex. In one aspect, the invention includes a method of screening candidate compounds for their ability to inhibit the binding of Vif to Cul5. The method includes contacting Vif and Cul5 in the presence of a candidate compound. Detecting inhibition or a reduced amount of Vif/Cul5 complex in the presence of the candidate compound compared to the amount of Vif/Cul5 complex in the absence of the candidate compound as an indication that the candidate compound is an inhibitor of Vif/Cul5 interaction.

In one embodiment, the compounds are useful in treating a disease, disorder, or condition associated with Vif. Preferably, the compounds are useful in treating HIV infection.

In another aspect, the invention includes a method for inhibiting interaction of Vif with Cul5 in a mammal, where decreasing interaction between Vif and Cul5 serves to treat a mammal suffering from a viral infection associated with Vif, preferably HIV. The method comprises administering a compound that inhibits the interaction between Vif and Cul5 to the mammal in need thereof. Preferably, the mammal is a human.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a schematic of vif in the context of its binding partners.

FIG. 2 is a schematic of representative constructs expressed from a vector that can coexpress target genes. The first target gene depicted comprises ElonginB and Vif. The second target gene comprises ElonginC. Linker Regions to express the EloB-Vif fusion protein in the presence of co-expressed EloC is depicted.

FIG. 3 is an image demonstrating purification of EloB/EloC/vif complexes.

FIG. 4 is an image depicting gel filtration analysis of EloB/EloC/vif complexes.

FIG. 5 is a schematic diagram of a vif-EloB/EloC-Cullin5-Rbx2 E3 ubiquitin ligase complex. Vif binds the substrates A3G and A3F. Recruitment of substrates to the E3 complex facilitates their poly-ubiquitination and ultimately proteasomal degradation.

FIG. 6 is a series of images depicting EloB/EloC/vif complexes binding to Cul5. FIG. 6 shows GST pulldown assays demonstrating the interaction between EloB/Vif/EloC and Cul5 is mediated through the HIV-1 protein Vif.

FIG. 7 is a image depicting isothermal titration calorimetry (ITC) demonstrating specific binding of Cul5N to the EloB/Vif/EloC complex. Data was fit to a one-site binding model; the measured equilibrium K_{D}=266 nM and the N value was 0.7.

FIG. 8 is a series of images depicting crystallization and X-ray diffraction of two EloB/Vif/EloC complexes.

FIG. 9 is an image supporting the presence of zinc in vif. Fluorescence scans around the zinc K absorbance edge were performed and are depicted for an EloB(1-104)/Vif(102-173)/EloC(17-112) crystal (blue), an EloB-linker-vif/EloC crystal (red), a small molecule crystal with zinc (orange), and a small molecule crystal without zinc (black). The increases in fluorescence near the zinc K absorption edge for both of the EloB/EloC/vif crystals supports the presence of zinc in vif.

FIG. 10, comprising FIGS. 10A-10D, is a series of images summarizing ITC analysis of the interaction between EloBC-vif complexes and Cul5(N). The 1:1 binding stoichiometry observed for the EloBCvif interaction with Cul5 is consistent with the binding stoichiometry of complexes comprising EloBC with cellular SOCS-box proteins and Cul5.

FIG. 11, comprising FIGS. 11A-11F, is a series of images of GST pull-down experiments showing that the interaction between EloBC-vif and Cul5 is mediated by vif. FIG. 11A depicts GST-Cul5(N)-bound to glutathione-resin (lane 1) and GST-bound glutathione-resin (lane 2). FIGS. 11B-11F depict purified EloBCvif or BC complexes used as input for the pull-down experiments (lane 1). The supernatant from the final wash of GST-Cul5(N)-bound resin after 2 hr incubation with input EloBCvif or BC complexes (lane 2). GST-Cul5(N)-bound glutathione-resin after washing away unbound EloBCvif or BC complexes (lane 3). GST-bound glutathione-resin after washing away unbound EloBC-vif or BC complexes (lane 4).

FIG. 12, comprising FIGS. 12A and 12B, is a series of images depicting Vif sequence and constructs. FIG. 12A depicts the amino acid sequence of SEQ ID NO: 12 with conserved motifs for the vif C-terminus of HIV-1 variant HXB-2. Conserved residues within the zinc-binding domain are bold; zinc ligands are purple; residues of the BC-box are orange; and residues of the dimerization motif are in blue. FIG. 12B is a schematic representation of each tripartite developed for this study. EloB is full-length human EloB. EloC is residues 17-112 of human EloC. All double-mutant pairs were introduced into the BCvif₉₅₋₁₉₂ complex. The A123S, L124S double mutant was also introduced into the BCvif₉₅₋₁₅₅ complex.

FIG. 13 is an image depicting representative size-exclusion chromatography profiles of several BC-vif complexes. 300 μL of ˜2.5 mg ml⁻¹ of purified EloBC-vif tripartite complexes or EloBC dimer were injected onto an S-100 sephacryl size-exclusion chromatography column with a flow rate of 0.25 ml min⁻¹ at 20° C. The molecular mass calibration standards, albumin (67 kD), ovalbumin (43 kD), Chymotrypsinogen (25 kD), and RNase A (13.7 kD) were injected under identical conditions and eluted with peak positions indicated by black arrows. The void volume elution time was 137.5 minutes.

FIG. 13, comprising FIGS. 13A-13C, is a series of images depicting purification of tripartite EloBCvif complexes and BC heterodimer. FIG. 13A depicts an SDS-PAGE analysis depicting various stages of purification of BCvif₉₅₋₁₉₂. The lanes are: 1) NiNTA-purified EloB-linker-vif₉₅₋₁₉₂/EloC; 2) eluted complex after cleavage with ProTEV protease; lane 3) Mᵣ markers with corresponding values in kDa; lane 4) Re-purification of the cleaved tripartite BCvif₉₅₋₁₉₂ complex after passing through Ni-NTA resin; lanes 5 to 14) Size-exclusion chromatography fractions for the elution peak of tripartite complex BCvif₉₅₋₁₉₂. All lanes were separated by 4-12% Bis-Tris gradient gels visualized with Coomassie Brilliant Blue. FIG. 13B is an image of an SDS-PAGE analysis depicting purification of BCvif₉₅₋₁₅₅ tripartite complex. The lanes are: 1) NiNTA-purified EloB-linker-vif₉₅₋₁₅₅/EloC eluted with Buffer E. 2) eluted protein complex after cleavage with 5 units ProTEV protease/milligram protein for 20 h at 4° C. 3) Tripartite BCvif₉₅₋₁₅₅ complex after re-incubation of cleaved material with Ni-NTA resin. 4-12) S-100 size-exclusion chromatography fractions encompassing the elution peak of tripartite complex BCvif₉₅₋₁₅₅. FIG. 13C is an image of an SDS-PAGE analysis depicting purity of tripartite EloBCvif complexes comprising wild type and mutant vif proteins of various length. To illustrate purity, fractions pooled from S-100 elution peaks were analyzed by SDS-PAGE. Each representative complex is greater than 95% pure as evaluated with Coomassie Brilliant Blue. Lanes labeled WT possess the wild-type vif moiety of the indicated length. SS, GA, and VF represent the mutations to A123 and L124 respectively. Mᵣ are commercially available molecular mass standards (Invitrogen and Fermentas), with molecular masses indicated (kDa). In several samples, EloC and vif moieties migrate to the same position (BCvif95-173, BCvif95-160, B104Cvif102-173).

FIG. 15 is an image demonstrating that the experimentally determined molecular mass (Mᵣ) of each purified EloBC-vif complex is greater than the protein-sequence calculated molecular mass of each tripartite complex. Experimentally determined Mw are calculated from the elution volume peak for each complex with the standard curve generated with molecular weight standards. The expected molecular mass was calculated for 1:1:1 molar ratio tripartite complexes or for the 1:1 molar ratio of dimeric BC complex from respective protein sequences. EloBCvif complexes were subjected to SEC; each EloBCvif tripartite complex eluted from the S-100 SEC column with an experimentally determined Mᵣ greater than the value expected for 1:1:1 molar ratio complexes, but lower than the value expected for 2:2:2 molar ratio complexes. The BC sample eluted with an experimentally determined Mᵣ proximal to that calculated for a 1:1 molar ratio heterodimer.

FIG. 16, comprising FIGS. 16A-16E, is a series of images demonstrating the characterization of oligomeric assembly of BC and BCvif complexes.

FIG. 17, comprising FIGS. 17A through 17M, is a series of images depicting the sequences of SEQ ID NO: 17-57.

### DETAILED DESCRIPTION OF THE INVENTION

The present invention is based, at least in part, on the ability to produce large amounts of recombinant Vif and Cul5 proteins. Preferably, the recombinant Vif and Cul5 proteins are soluble. The Vif and Cul5 proteins are useful for use in screening assays to identify agents that are able to inhibit the interaction between Vif and Cul5. In one embodiment, the assay of the invention is able to screen for an agent that inhibits the ability of Vif to bind with Cul5. Without wishing to be bound by any particular theory, inhibiting Vif binding to Cul5 also inhibits recruitment of E3 ligase, which is thought to be an important component of the cellular ubiquitin protease machinery. The agent can target a domain in the Vif protein that is required for the interaction with Cul5. In some instances, the agent can target a domain in the Cul5 protein that is required for the interaction with Vif. In another instance, the agent can target a domain in the Vif protein and a domain in the Cul5 protein.

The present invention provides a Vif-mediated assay and agents identified by the Vif-mediated assay. Accordingly, the invention provides a method of preventing ubiquitination of APOBEC3G without broadly inhibiting the cell's ability to carry out ubiquitination on other proteins. In other words, the invention demonstrates a means of creating ubiquitination scaffolds/complexes whose assembly are dependent on Vif and therefore enable for the first time the screening for compounds that have antiviral activity based on their ability to disrupt Vif dependent ubiquintination of APOBEC3G.

Inhibiting or reducing the interaction between Vif and Cul5 allows the virus to become sensitive to the antiviral activities of Apobec3G (or one of the other noted Apobec 3 proteins). Therefore, if a cell that is producing virus is treated with an agent that inhibits Vif and Cul5 interaction, virus that is being produced by the cell is inactivated and thus is unable (or exhibits a reduced capacity) to carry out future rounds of infection. In this manner, infectivity of the virus is inhibited by the compounds identified by the screening methods of the invention.

The method disclosed herein allows for rapid screening of agents for their ability to inhibit interaction between Vif and Cul5, which agents are important potential therapeutics for use in methods where selectively inhibiting of Vif and Cul5 binding provides a therapeutic benefit, including, but not limited to, development of agents useful for treating viral infection, while reducing the risk of cell toxicity that might otherwise arise form general inhibitors of ubiquitination Preferably, the viral infection is HIV.

### DEFINITIONS

As used herein, each of the following terms has the meaning associated with it in this section.

The articles “a” and “an” are used herein to refer to one or to more than one (e.g., to at least one) of the grammatical object of the article. By way of example, “an element” means one element or more than one element.

The term “binding” refers to a direct association between at least two molecules, due to, for example, covalent, electrostatic, hydrophobic, ionic and/or hydrogen-bond interactions under physiological conditions.

A “fusion protein” is a fusion of a first amino acid sequence encoded by a polynucleotide with a second amino acid sequence defining a domain foreign to and not substantially homologous with any domain of the first amino acid sequence.

As used herein, the term “fragment,” as applied to a nucleic acid, refers to a subsequence of a larger nucleic acid. A “fragment” of a nucleic acid can be at least about 20 nucleotides in length; for example, at least about 50 nucleotides to about 100 nucleotides; preferably at least about 100 to about 500 nucleotides, more preferably at least about 500 to about 1000 nucleotides, even more preferably at least about 1000 nucleotides to about 1500 nucleotides; particularly, preferably at least about 1500 nucleotides to about 2500 nucleotides; most preferably at least about 2500 nucleotides.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., retroviruses, lentiviruses, adenoviruses, and adeno-associated viruses) that incorporate the recombinant polynucleotide.

As used herein, the term “gene” refers to an element or combination of elements that are capable of being expressed in a cell, either alone or in combination with other elements. In general, a gene comprises (from the 5′ to the 3′ end): (1) a promoter region, which includes a 5′ nontranslated leader sequence capable of functioning in any cell such as a prokaryotic cell, a virus, or a eukaryotic cell (including transgenic mammals); (2) a structural gene or polynucleotide sequence, which codes for the desired protein; and (3) a 3′ nontranslated region, which typically causes the termination of transcription and the polyadenylation of the 3′ region of the RNA sequence. Each of these elements is operatively linked by sequential attachment to the adjacent element. A gene comprising the above elements is inserted by standard recombinant DNA methods into any expression vector.

As used herein, “gene products” include any product that is produced in the course of the transcription, reverse-transcription, polymerization, translation, post-translation and/or expression of a gene. Gene products include, but are not limited to, proteins, polypeptides, peptides, peptide fragments, or polynucleotide molecules.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 5′TATGGC3′ share 50% homology.

As used herein, “homology” is used synonymously with “identity.”

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. Moreover, an “isolated” nucleic acid molecule can be substantially free of other cellular material, or culture medium when produced by recombinant techniques, or substantially free of chemical precursors or other chemicals when chemically synthesized.

As used herein, an “inhibitory-effective amount” is an amount that results in a detectable (e.g., measurable) amount of inhibition of an activity of Vif, such as its ability to target and degrade A3G in a cell infected by a lentivirus. In some instance, the activity of Vif is its ability to bind with Cullin5.

The term “lentivirus” as used herein may be any of a variety of members of this genus of viruses. In one embodiment of the invention, the lentivirus contains a Vif gene. The lentivirus may be, e.g., one that infects a mammal, such as a sheep, goat, horse, cow or primate, including human. Typical such viruses include, e.g., Vizna virus (which infects sheep); simian immunodeficiency virus (SIV), bovine immunodeficiency virus (BIV), chimeric simian/human immunodeficiency virus (SHIV), feline immunodeficiency virus (FIV) and human immunodeficiency virus (HIV). “HIV,” as used herein, refers to both HIV-1 and HIV-2. Much of the discussion herein is directed to HIV or HIV-1; however, it is to be understood that other suitable lentiviruses are also included.

The term “mammal” as used herein refers to any non-human mammal. Such mammals are, for example, rodents, non-human primates, sheep, dogs, cows, and pigs. The preferred non-human mammals are selected from the rodent family including rat and mouse, more preferably mouse. The preferred mammal is a human.

A “nucleic acid molecule” is intended generally to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids which can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptide, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “polynucleotide” includes cDNA, RNA, DNA/RNA hybrid, anti-sense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gene, including but not limited to deletion, insertion, substitution of one or more nucleotides, or fusion to other polynucleotide sequences, provided that such changes in the primary sequence of the gene do not alter the expressed peptide ability to elicit passive immunity.

“Pharmaceutically acceptable” means physiologically tolerable, for either human or veterinary applications. In addition, “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. Essentially, the pharmaceutically acceptable material is nontoxic to the recipient. The carrier would naturally be selected to minimize any degradation of the active ingredient and to minimize any adverse side effects in the subject, as would be well known to one of skill in the art. For a discussion of pharmaceutically acceptable carriers and other components of pharmaceutical compositions, see, e.g., Remington's Pharmaceutical Sciences, 18th ed., Mack Publishing Company, 1990.

As used herein, “pharmaceutical compositions” include formulations for human and veterinary use.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

A “recombinant nucleic acid” is any nucleic acid that has been placed adjacent to another nucleic acid by recombinant DNA techniques. A “recombined nucleic acid” also includes any nucleic acid that has been placed next to a second nucleic acid by a laboratory genetic technique such as, for example, transformation and integration, transposon hopping or viral insertion. In general, a recombined nucleic acid is not naturally located adjacent to the second nucleic acid.

The term “recombinant protein” refers to a protein of the present invention which is produced by recombinant DNA techniques, wherein generally DNA encoding the expressed protein is inserted into a suitable expression vector which is in turn used to transform a host cell to produce the heterologous protein. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein.

“Test agents” or otherwise “test compounds” as used herein refers to an agent or compound that is to be screened in one or more of the assays described herein. Test agents include compounds of a variety of general types including, but not limited to, small organic molecules, known pharmaceuticals, polypeptides; carbohydrates such as oligosaccharides and polysaccharides; polynucleotides; lipids or phospholipids; fatty acids; steroids; or amino acid analogs. Test agents can be obtained from libraries, such as natural product libraries and combinatorial libraries. In addition, methods of automating assays are known that permit screening of several thousands of compounds in a short period.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

“Viral infectivity” as that term is used herein means any of the infection of a cell, the replication of a virus therein, and the production of progeny virions therefrom.

A “virion” is a complete viral particle; nucleic acid and capsid, further including and a lipid envelope in the case of some viruses.

### DESCRIPTION

The present invention is based on the discovery that recombinant Vif and Cullin5 can be produced in a large-scale quantity sufficient for at least in vitro studies. The disclosure presented herein demonstrates that recombinant Vif can be produced in a soluble form when Vif is in the context of an ElonginB/ElonginC/Vif complex. Importantly, the ElonginB/ElonginC/Vif complex comprises a binding region for Cullin5 to bind with Vif. The biological significance is that the resulting proteins form an interaction network that allows for assessing and high throughput screening of Vif-mediated and selective interaction between Cullin5 and ElonginB/C. Without wishing to be bound by any particular theory, the present method of assessing Vif-mediated interaction between Cullin5 and ElonginB/C allows for assessing the activity of the Cullin-RING E3 ligase which is responsible for degradation of the innate antiviral proteins such as APOBEC3G (A3G) and APOBEC3F (A3F).

The unique design of the recombinant proteins enables the ability to generate ElonginB/ElonginC/Vif as well as Cullin5 in soluble forms in a large-scale production setting allows for the development of an assay to screen for agents that block the Vif interaction with Cullin5. The assay provides a method to screen for any agent that inhibits the ability of Vif bind with Cul5. The agent can target a domain in the Vif protein, Cul5 protein, or both whereby interaction between Vif and Cul5 is inhibited.

The invention is also based on the discovery that ElonginB, ElonginC, Vif and Cullin5 can be produced in a large-scale quantity and purified in large quantities. These proteins can form stable Elognin B/C-Vif-Cullin5 that are suitable as starting material for high throughput screening assays.

### Compositions

The present invention relates to compositions that are useful for screening for agents that inhibit binding between Vif and Cul5. The invention is partly based on the discovery of the macromolecular composition that enables the large-scale production of Vif and Cul5 can be produced recombinantly. The large amount of recombinant protein produced allows for the use of these proteins in a screening assay to identify agents that are able to inhibit the interaction between Vif and Cul5.

Large amounts of Vif can be produced by cloning a Vif/ElonginB fusion protein containing a flexible and cleavable linker between Elongin B and Vif. For example, the relevant interacting domain of Vif can be fused to the C-terminus of Elongin B thereby generating the Vif/ElonginB fusion protein. In a preferred embodiment, the Vif/ElonginB fusion protein is also co-expressed with Elongin C from a single vector. As a non-limiting example, a vector that can express the Vif/Elongin B and Elogin C transcript is the pETDuet expression vector. However, any vector that can coexpress two target genes can be used to generate the two polypeptides. For example, such a vector can comprise two multiple cloning sites (MCS) each of which is preceded by a promoter. However, it is envisioned that ElonginB/ElonginC/Vif may not need to by expressed from one vector. Rather, each protein can be produced separately and the proteins can be subsequently combined to generate the ElonginB/ElonginC/Vif tripartite complex.

In one embodiment, large quantities of soluble Vif are produced by engineering a fusion comprising the Cullin5 Box domain of Vif (amino acids 95-173; SEQ ID NO: 9) with the C-terminal polypeptide chain of ElonginB (amino acids 1-118; SEQ ID NO: 10). The Vif/ElonginB transcript is co-expressed with ElonginC from a single vector. Preferably, amino acids 17-122 of EloninC (SEQ ID NO: 2) is used to generate the protein complex. In this context, ElonginC is the partner protein for Vif/ElongB thereby allowing for solubilization of the fusion protein complex. The resulting bi-polypeptide complex is processed by protease cleavage to remove a flexible linker that joined the C-terminus of ElonginB and the N-terminus of the Vif construct. The resulting tripartite complex of ElonginB/ElonginC/Vif remains folded as a compact globular shape in solution.

In another embodiment, any sequence corresponding to Vif can be used to generate an ElonginB/Vif fusion protein. Preferably, the Vif sequence comprises sequences corresponding to the Cullin5 Box domain. For example, Vif (amino acids 95-173; SEQ ID NO: 9) or Vif (amino acids 102-173; SEQ ID NO: 14) can be used.

In another embodiment, any sequence corresponding to ElonginB can be used to generate an ElonginB/Vif fusion protein. For example, ElonginB (amino acids 1-118; SEQ ID NO: 10), ElonginB (amino acids 1-104; SEQ ID NO: 12), or ElonginB (amino acids 1-98; SEQ ID NO: 13) can be used.

In order to evaluate the Vif-mediated interaction between Cul5 and ElonginB/ElonginC, an N-terminal domain of Cullin5 (amino acids 1-384; SEQ ID NO: 11) that encompasses the Vif interaction domain was generated. To improve solubility, two point mutations were introduced within the Cul5 fragment. The point mutations are V341R and L345D. For purification purposes, a reporter or tag can be engineered to the Cul5 fragment.

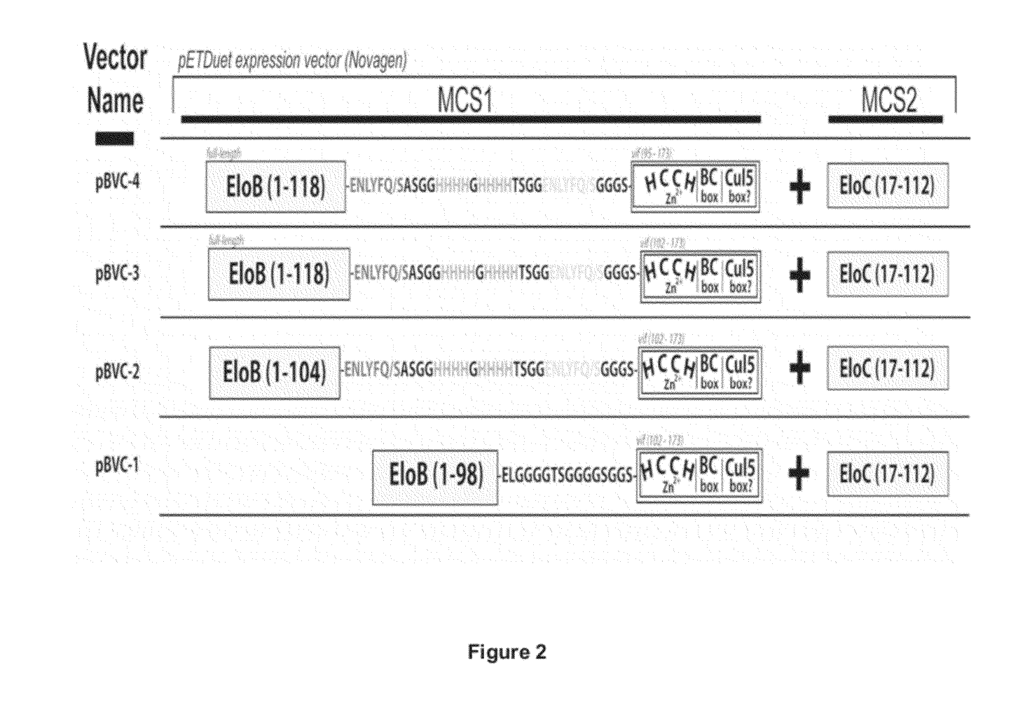

The present disclosure is the first time large-scale production of Vif, ElonginB, ElonginC, Cul5 has been successfully demonstrated. The level of protein production is suitable for at least in vitro high-throughput screening. Based on the information provided herein, the polypeptides of the invention can be produced recombinantly using standard techniques well known to those of skill in the art or produced by a host cell. For example, the sequences of Vif, ElonginB, ElonginC, Cul5 are known and can be used to engineer the polypeptides of the invention (e.g., FIG. 2). The nucleic acid sequence may be optimized to reflect particular codon “preferences” for various expression systems according to methods known in the art.

In general, the fusion polypeptides of the invention can be produced by preparing a fused gene comprising a first DNA segment and a second DNA segment. Each fused gene is assembled in, or inserted into an expression vector. Recipient cells capable of expressing the gene products are then transfected with the genes. The transfected recipient cells are cultured under conditions that permit expression of the incorporated genes and the expressed and fusion proteins are harvested.

Using the sequence information provided herein, the nucleic acids may be synthesized according to a number of standard methods known in the art. Oligonucleotide synthesis, is carried out on commercially available solid phase oligonucleotide synthesis machines or manually synthesized using the solid phase phosphoramidite triester method described by Beaucage et. al., 1981 Tetrahedron Letters. 22: 1859-1862.

Once a nucleic acid encoding a the desired polypeptide is synthesized, it may be amplified and/or cloned according to standard methods in order to produce recombinant polypeptides. Molecular cloning techniques to achieve these ends are known in the art. A wide variety of cloning and in vitro amplification methods suitable for the construction of recombinant nucleic acids are known to those skilled in the art.

Examples of techniques sufficient to direct persons of skill through in vitro amplification methods, including the polymerase chain reaction (PCR), the ligase chain reaction (LCR), and other DNA or RNA polymerase-mediated techniques are found in Sambrook et al., MOLECULAR CLONING: A LABORATORY MANUAL, volumes 1-3 (3rd ed., Cold Spring Harbor Press, NY 2001).

Once the nucleic acid for a desired polypeptide is cloned, a skilled artisan may express the recombinant gene(s) in a variety of engineered cells. Examples of such cells include bacteria, yeast, filamentous fungi, insect (especially employing baculoviral vectors), and mammalian cells. It is expected that those of skill in the art are knowledgeable in the numerous expression systems available for expressing the polypeptides of the invention.

The present invention also provides for analogs of polypeptides of the invention. Analogs may differ from naturally occurring proteins or polypeptides by conservative amino acid sequence differences or by modifications which do not affect sequence, or by both. For example, conservative amino acid changes may be made, which although they alter the primary sequence of the protein or polypeptide, do not normally alter its function (e.g., secretion and capable of blocking virus infection). Conservative amino acid substitutions typically include substitutions within the following groups: (a) glycine, alanine; (b) valine, isoleucine, leucine; (c) aspartic acid, glutamic acid; (d) asparagine, glutamine; (e) serine, threonine; (f) lysine, arginine; (g) phenylalanine, tyrosine.

Modifications (which do not normally alter primary sequence) include in vivo, or in vitro, chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

The present invention should also be construed to encompass “mutants,” “derivatives,” and “variants” of the peptides of the invention (or of the DNA encoding the same) which mutants, derivatives and variants are altered in one or more amino acids (or, when referring to the nucleotide sequence encoding the same, are altered in one or more base pairs) such that the resulting peptide (or DNA) is not identical to the sequences recited herein, but has the same biological property as the polypeptides disclosed herein, in that the peptide has biological/biochemical properties.

### Vectors

Nucleic acids encoding the desired polypeptide or equivalents may be replicated in wide variety of cloning vectors in a wide variety of host cells.

In brief summary, the expression of natural or synthetic nucleic acids encoding a desired polypeptide will typically be achieved by operably linking a nucleic acid encoding the desired polypeptide or portions thereof to a promoter, and incorporating the construct into an expression vector. The vectors can be suitable for replication and integration. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors. In some aspects, the expression vector is selected from the group consisting of a viral vector, a bacterial vector, and a mammalian cell vector. Numerous expression vector systems exist that comprise at least a part or all of the compositions discussed above. Prokaryote- and/or eukaryote-vector based systems can be employed to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al., MOLECULAR CLONING: A LABORATORY MANUAL, volumes 1-3 (3rd ed., Cold Spring Harbor Press, NY 2001), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (e.g., WO 01/96584; WO 01/29058; and U.S. Pat. No. 6,326,193).

For expression of the polypeptides of the invention or portions thereof, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” e.g., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (e.g., U.S. Pat. No. 4,683,202, U.S. Pat. No. 5,928,906).

Naturally, it will be important to employ a promoter and/or enhancer that effectively directs the expression of the DNA segment in the cell type, organelle, and organism chosen for expression. The promoters employed may be constitutive, tissue-specific, inducible, and/or useful under the appropriate conditions to direct high level expression of the introduced DNA segment, such as is advantageous in the large-scale production of recombinant proteins and/or polypeptides. The promoter may be heterologous or endogenous.

An example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of the polypeptides of the invention or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

The invention includes a tag polypeptide that can be covalently linked thereto to the polypeptides of the invention. That is, the invention encompasses a recombinant nucleic acid wherein the nucleic acid encoding the tag polypeptide is covalently linked to the nucleic acid of the polypeptides of the invention. Such tag polypeptides are well known in the art and include, for instance, green fluorescent protein (GFP), myc, myc-pyruvate kinase (myc-PK), His₆, maltose binding protein (MBP), an influenza virus hemagglutinin tag polypeptide, a flag tag polypeptide (FLAG), a glutathione-S-transferase (GST) tag polypeptide, and a EGFP protein. However, the invention should in no way be construed to be limited to the nucleic acids encoding the above-listed tag polypeptides. Rather, any nucleic acid sequence encoding a polypeptide which may function in a manner substantially similar to these tag polypeptides should be construed to be included in the present invention. Further, addition of a tag polypeptide facilitates isolation and purification of the “tagged” protein such that the protein of the invention can be produced and purified readily.

### Methods of Introduction and Expression

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., MOLECULAR CLONING: A LABORATORY MANUAL, volumes 1-3 (3rd ed., Cold Spring Harbor Press, NY 2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape.

Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Chol”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol.

“Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Certain post-translational modifications are the result of the action of recombinant host cells on the expressed polypeptide. Glutaminyl and aspariginyl residues are frequently post-translationally deamidated to the corresponding glutamyl and aspartyl residues. Alternatively, these residues are deamidated under mildly acidic conditions. Either form of these residues falls within the scope of this invention.

Other post-translational modifications include hydroxylation of proline and lysine, phosphorylation of hydroxyl groups of seryl, threonyl or tyrosyl residues, methylation of the α-amino groups of lysine, arginine, and histidine side chains (T E Creighton (1983) Proteins: Structure and Molecular Properties, W.H. Freeman & Co., San Francisco, pp. 79-86).

Regardless of the method used to introduce exogenous nucleic acids into a host cell or otherwise expose a cell to the nucleic acid, in order to confirm the presence of the recombinant DNA sequence in the host cell, a variety of assays may be performed. Such assays include, for example, “molecular biological” assays well known to those of skill in the art, such as Southern and Northern blotting, reverse transcription polymerase chain reaction (RT-PCR) and PCR; “biochemical” assays, such as detecting the presence or absence of a particular peptide, e.g., by immunological means (ELISAs and Western blots).

### Screening Assay

As a non-limiting example, a 71 amino acid domain of Vif was produced as a fusion protein with human Elongin B, and co-expressed with human Elongin C to produce milligram quantities of soluble and functional protein. Moreover, the production of a soluble Cullin5 protein, sufficient to interact with Vif, provides an opportunity to initiate in vitro drug discovery assays. The assay are useful for identifying antiviral compounds that selectively bind to Vif or selectively inhibit Vif-mediated interactions between Cullin 5 and EloB/C, which is necessary for ubiquitination and degradation of the innate antiviral proteins APOBEC3G and APOBEC3F. The assays described here are unique and are an enabling technology for the HIV/AIDS drug discovery industry.

The invention provides a method for identifying compounds that bind to Vif when the ElonginB-Vif chimera or the interacting domain of Vif are used in assays to contact chemistries from a library of compounds. In such instances, ElonginB-Vif chimera or the interacting domain of Vif with an appropriate tag (such as GST, poly Histine or epitope tag etc) would be immobilized on a solid support and interacted with compounds with chemical libraries with the expressed intent of identify those compounds that bound to Vif.

The invention provides a method of identifying compounds that block the interaction between Cul5 and Vif. Without wishing to be bound by any particular theory, it is believed that blocking the interaction between Cul5 and Vif protects the infected cell's own innate immune factors, such as A3G and A3F. That is, under normal circumstances, HIV-1 infection leads to the production of the viral protein Vif. This viral factor is essential to evade the host's own A3G and A3F defense factors. Vif works in concert with the cell's own ubiquitin ligase machinary to promote degradation of A3G and A3F by the 26S proteasome. This requires a direct interaction between the HIV-1 protein Vif and Cullin 5.

Accordingly, the invention includes compounds identified by the screening methods that block the virus/host protein interaction. These compounds are considered antiviral compounds because they prevent Vif-APOBEC3G or Vif-APOBEC3F from interacting with Culin 5 and the other components of the ubiquitination machinery. Consequently APOBEC3G and APOBEC3F are not destroyed by Vif and the increased intracellular abundance of these host-defense factors enables them to enter nascent viral particles from which point the host-defense factors are positioned to interact with viral replication complexes and assemble with viral particles following infection and thereby block viral infectivity.

One aspect of the invention is a method for identifying an agent (e.g. screening putative agents for one or more that elicits the desired activity) that inhibits the infectivity of a lentivirus (e.g., a lentivirus which expresses a Vif protein). Typical such lentiviruses include, e.g., SW, SHIV and/or HIV. The method takes advantage of the successful production of large-scale amounts of recombinant Vif and Cul5 proteins. These proteins allow for assays for detecting an agent that is capable of interfering with the interaction between Vif and Cul5. An agent that interferes with Vif/Cul5 complex would be expected to inhibit infectivity of a lentivirus that expresses a Vif protein. Furthermore, because the assay is Vif-mediated or otherwise Vif dependent (Vif is not found in other cellular proteins), such an agent would not be expected to interfere with the function of cellular proteins and thus would be expected to elicit few, if any, side effects as a result of the binding.

The method comprises: (a) contacting a putative inhibitory agent with a mixture comprising Vif and Cul5 under conditions that are effective for Vif/Cul5 complex formation; and (b) detecting whether the presence of the agent decreases the level of Vif/Cul5 complex formation. In some instances, the agent binds to Vif and thereby inhibits Vif/Cul5 complex formation. In another instance, the agent binds to Cul5 and thereby inhibits Vif/Cul5 complex formation. Any of a variety of conventional procedures can be used to carry out such an assay.

The invention encompasses methods to identify a compound that inhibits the interaction between Vif and Cul5. In one embodiment, the invention provides an assay for determining the binding between Cul5 with Vif, wherein Vif is in the context of ElonginB/ElonginC/Vif complex. The method includes contacting recombinant Vif and Cul5 in the presence of a candidate compound. Detecting inhibition or a reduced amount of Vif/Cul5 complex in the presence of the candidate compound compared to the amount of Vif/Cul5 complex in the absence of the candidate compound is an indication that the candidate compound is an inhibitor of Vif/Cul5 interaction.

Based on the disclosure presented herein, the screening method of the invention is applicable to a robust Förster quenched resonance energy transfer (FqRET) assay for high-throughput compound library screening in microtiter plates. The assay is based on selective placement of chromoproteins or chromophores that allow reporting on complex formation between the EloB/C/Vif protein and EloC in vitro. For example, an appropriately positioned FRET donor and FRET quencher will results in a “dark” signal when the quaternary complex is formed between Vif and Cullin5. However, the screening methods should not be limited solely to the assays disclosed herein. Rather, the recombinant proteins of the invention can be used in any assay, including other high-throughput screening assays, that are applicable for screening agents that regulate the binding between to proteins. Thus, the invention encompasses the use of the recombinant proteins of the invention in any assay that is useful for detecting an agent that interferes with protein-protein interaction. Furthermore, the screening methods should also not be limited to Cullin5-Vif antagonist as Vif-ElonginB antogonist and Vif-ElonginC antongist are also envisioned and these would also have value as to antiviral compounds that prevent APOBEC3G/3F ubiquitination and degradation.

The skilled artisan would also appreciate, in view of the disclosure provided herein, that standard binding assays known in the art, or those to be developed in the future, can be used to assess the binding of Vif with Cul5 using the recombinant proteins of the invention in the presence or absence of the test compound to identify a useful compound. Thus, the invention includes any compound identified using this method.

The screening method includes contacting a mixture comprising recombinant Vif and Cul5 with a test compound and detecting the presence of the Vif/Cul5 complex, where a decrease in the level of Vif/Cul5 complex compared to the amount in the absence of the test compound or a control indicates that the test compound is able to inhibit the binding between Vif and Cul5. In certain embodiments, the control is the same assay performed with the test compound at a different concentration (e.g. a lower concentration), or in the absence of the test agent, etc.

Without wishing to be bound by any particular theory, it is believed that the Vif/Cul5 complex contains a ceiling level of complex formation because the presence the two proteins have a propensity to bind with one another and in the absence of the cullin5 scaffold, E2 ligase cannot ubiquinate Vif or APOBEC3G/3F and thus Vif will not mediate the distruction of APOBEC3G/3F. The activity of a test compound can be measured by determining whether the test compound can decrease the level of Vif/Cul5 complex formation.

Determining the ability of the test compound to interfere with the formation of the Vif/Cul5 complex, can be accomplished, for example, by coupling the Vif protein or the Cul5 protein with a tag, radioisotope, or enzymatic label such that the Vif/Cul5 complex can be measured by detecting the labeled component in the complex. For example, a component of the complex (e.g., Vif or Cul5) can be labeled with ³²P, ¹²⁵I, ³⁵S, ¹⁴C, or ³H, either directly or indirectly, and the radioisotope detected by direct counting of radioemission or by scintillation counting. Alternatively, a component of the complex can be enzymatically labeled with, for example, horseradish peroxidase, alkaline phosphatase, or luciferase, and the enzymatic label is then detected by determination of conversion of an appropriate substrate to product.

Determining the ability of the test compound to interfere with the Vif/Cul5 complex can also be accomplished using technology such as real-time Biomolecular Interaction Analysis (BIA) as described in Sjolander et al., 1991, Anal. Chem. 63:2338-2345 and Szabo et al., 1995, Curr. Opin. Struct. Biol. 5:699-705. BIA is a technology for studying biospecific interactions in real time, without labeling any of the interactants (e.g., BIAcore, BIAcore International AB, Uppsala, Sweden). Changes in the optical phenomenon of surface plasmon resonance (SPR) can be used as an indication of real-time reactions between biological molecules.

In more than one embodiment of the methods of the present invention, it may be desirable to immobilize either Vif or Cul5 to facilitate separation of complexed from uncomplexed forms of one or both of the molecules, as well as to accommodate automation of the assay. The effect of a test compound on the Vif/Cul5 complex, can be accomplished using any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/target fusion proteins can be adsorbed onto glutathione sepharose beads (Sigma Chemical, St. Louis, Mo.) or glutathione-derivatized micrometer plates, which are then combined with the other corresponding component of the Vif/Cul5 complex in the presence of the test compound. The mixture is incubated under conditions conducive to complex formation (e.g., at physiological conditions for salt and pH). Following incubation, the beads or microtiter plate wells are washed to remove any unbound material, the matrix is immobilized in the case of beads, and the formation of the complex is determined either directly or indirectly, for example, as described above.

Other techniques for immobilizing proteins on matrices can also be used in the screening assays of the invention. For example, either Vif or Cul5 can be separated from a mixture using conjugated biotin and streptavidin. For example, biotinylated Cul5 or Vif can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates. Alternatively, antibodies reactive with for example Cul5 or Vif, but which do not interfere with binding of Vif with Cul5 can be derivatized to the wells of the plate. Methods for detecting such complexes, in addition to those described above for the GST-immobilized complexes, include immunodetection of complexes using reactive antibodies, as well as enzyme-linked assays.

The test compounds can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam et al., 1997, Anticancer Drug Des. 12:45).

Examples of methods for the synthesis of molecular libraries can be found in the art, for example, in: DeWitt et al., 1993, Proc. Natl. Acad. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria (Ladner U.S. Pat. No. 5,223,409), spores (Ladner U.S. Pat. No. '409), plasmids (Cull et al., 1992, Proc. Natl. Acad. Sci. USA 89:1865-1869) or on phage (Scott and Smith, 1990, Science 249:386-390; Devlin, 1990, Science 249:404-406; Cwirla et al., 1990, Proc. Natl. Acad. Sci. USA 87:6378-6382; Felici, 1991, J. Mol. Biol. 222:301-310; and Ladner supra).

In situations where “high-throughput” modalities are preferred, it is typical to that new chemical entities with useful properties are generated by identifying a chemical compound (called a “lead compound”) with some desirable property or activity, creating variants of the lead compound, and evaluating the property and activity of those variant compounds. The current trend is to shorten the time scale for all aspects of drug discovery.

In one embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

### Methods of Treatment

In one embodiment, the present invention provides methods of treating a disease, disorder, or condition associated with a viral infection. Preferably, the viral infection is associated with Vif, more preferably, the viral infection is HIV. The method comprises administering to a subject, such as a mammal, preferably a human, a therapeutically effective amount of a pharmaceutical composition that inhibits the interaction between Vif and Cullin5.

The invention includes compounds identified using the screening methods discussed elsewhere herein. Such a compound can be used as a therapeutic to treat an HIV infection or otherwise a disorder associated with Vif.

The ability for a compound to inhibit the interaction between Vif and Cullin5 can provide a therapeutic to protect or otherwise prevent viral infection, for example HIV infection.

Thus, the invention includes pharmaceutical compositions. Pharmaceutically acceptable carriers that are useful include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey), the disclosure of which is incorporated by reference as if set forth in its entirety herein.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic peritoneally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for oral, rectal, vaginal, peritoneal, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

The compositions of the invention may be administered via numerous routes, including, but not limited to, oral, rectal, vaginal, peritoneal, topical, pulmonary, intranasal, buccal, or ophthalmic administration routes. The route(s) of administration will be readily apparent to the skilled artisan and will depend upon any number of factors including the type and severity of the disease being treated, the type and age of the veterinary or human patient being treated, and the like.

As used herein, “peritoneal administration” of a pharmaceutical composition includes any route of administration characterized by physical breaching of a tissue of a subject and administration of the pharmaceutical composition through the breach in the tissue. Peritoneal administration thus includes, but is not limited to, administration of a pharmaceutical composition by injection of the composition, by application of the composition through a surgical incision, by application of the composition through a tissue-penetrating non-surgical wound, and the like. In particular, peritoneal administration is contemplated to include, but is not limited to, subcutaneous, intraperitoneal, intramuscular, intrasternal injection, and kidney dialytic infusion techniques.

A pharmaceutical composition can consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

Controlled- or sustained-release formulations of a pharmaceutical composition of the invention may be made using conventional technology.

Formulations of a pharmaceutical composition suitable for peritoneal administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for peritoneal administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for peritoneal administration, the active ingredient is provided in dry (i.e., powder or granular) form for reconstitution with a suitable vehicle (e.g., sterile pyrogen-free water) prior to peritoneal administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic peritoneally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Typically, dosages of the compound of the invention which may be administered to an animal, preferably a human, will vary depending upon any number of factors, including but not limited to, the type of animal and type of disease state being treated, the age of the animal and the route of administration.

The compound can be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, and the like. Preferably, the compound is, but need not be, administered as a bolus injection that provides lasting effects for at least one day following injection. The bolus injection can be provided intraperitoneally.

### EXAMPLES

The invention is now described with reference to the following Examples. These Examples are provided for the purpose of illustration only, and the invention is not limited to these Examples, but rather encompasses all variations which are evident as a result of the teachings provided herein.

The examples presented therein demonstrate a method of assaying for agents that are useful for treating HIV invention. CD4⁺ T cells, the primary target for HIV-1 infectivity, express the protein APOBEC3G, a cytidine deaminase that possesses an innate anti-retroviral activity. The enzyme catalyzes sequence-specific dC-to-dU deamination on negative-polarity single-stranded HIV-1 reverse transcripts. This reaction produces G-to-A mutations of the positive polarity (coding) strand of HIV-1 genomic RNA and coincides subsequently with diminished viral infectivity.

HIV-1 suppresses the innate antiretroviral activity of A3G with its protein vif (viral infectivity factor), a diminutive 23 kD essential accessory protein. Vif functions by directly binding A3G and recruiting it to a Cullin-RING E3 ubiquitin ligase (CRL) for ubiquitination and subsequent proteasomal degradation. This CRL comprises the cellular proteins Cullin5 (Cul5), ElonginB, ElonginC (EloB/C), and Rbx2. Acting as a substrate receptor for the CRL, vif binds A3G with N-terminal residues and makes critical contacts with EloB/C, and Cul5 through conserved C-terminal motifs. Essentially, vif tricks the cell into destroying its own antiretroviral protein just when it is needed most.

Several truncated forms of vif have been designed and recombinantly co-expressed with EloB/C to form highly purified tripartite complexes. These complexes are capable of binding recombinantly expressed Cul5 in a Vif-dependent manner, suggesting they possess biological functionality and are thus good candidates for high throughput screening (drug targets) and crystallization. Several small crystals of these EloB/C-vif complexes have been grown that display Bragg diffraction upon exposure to x-rays. The results presented herein demonstrate that the compositions and methods of the invention are useful for producing superior crystals of vif and cul5 suitable for structure determination.

### Example 1

### Largescale Production of Recombinant Elongin B, Elongin C, Cullin 5 and the HIV-1 Protein Vif

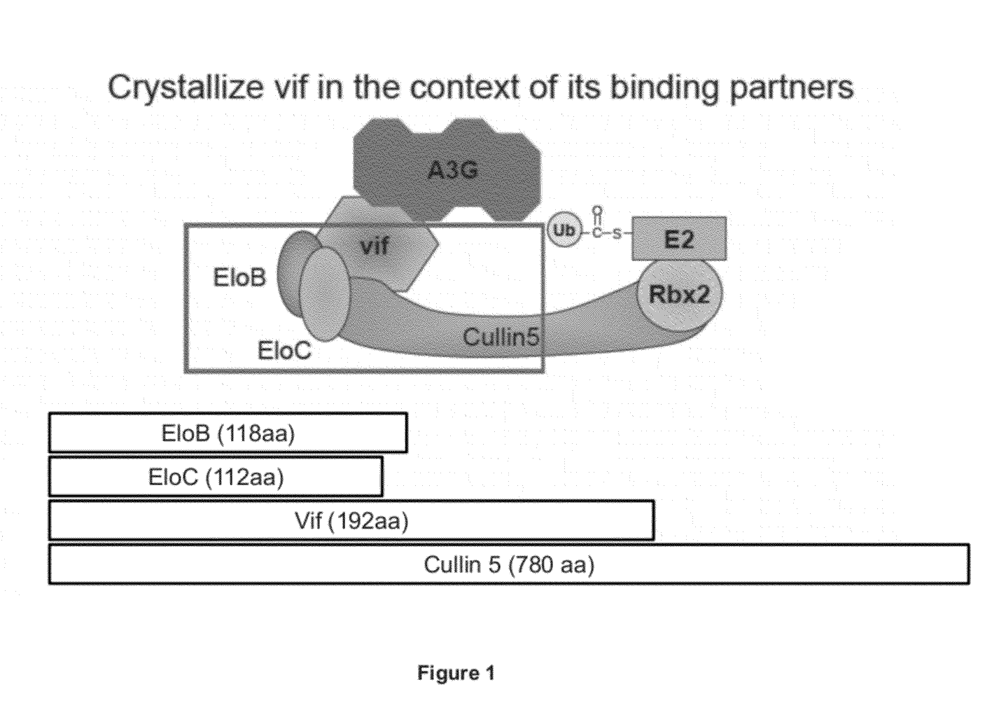

Experiments were designed to produce various recombinant proteins associated with Vif. For example, Elongin B, Elongin C, Cullin 5 and the HIV-1 protein Vif were produced on a milligram scale (FIG. 1). The present invention is the first time these proteins have been produced on such a large-scale. Large-scale production of these proteins is significant because the production of HIV-1 Vif in large quantities has been a major hurdle in the field. The large-scale production of biologically active Vif offers the ability to establish an in vitro high-throughput screening method aimed at identifying antiviral compounds.

When expressed on its own as an isolated protein, HIV-1 Vif protein is insoluble or aggregative, rendering production of quantities of this protein difficult. In order to produce large quantities of soluble Vif, a structure-guided design method was used to generate the Cullin5 Box domain of Vif in the context of a the C-terminal polypeptide chain (amino acids 95-173) fused to ElonginB (amino acids 1-118). The ElonginC partner protein is also co-expressed for solubilization and is required due to its interaction with Vif via a SOCS Box-like motif (FIG. 2). The resulting bi-polypeptide complex can be processed by TEV protease to remove a flexible linker that joins the C-terminus of ElonginB and the N-terminus of the Vif construct.

An N-terminal domain of Cullin5 (amino acids 1-384) that encompasses the Vif interaction domain was also generated. To improve solubility and purification, this protein was made as a GST-fusion protein. Two point mutants were utilized within the Cullin5 fragment to increase the solubility; these are V341R and L345D.

One novel technical innovation of the method discussed herein is the production of a Vif fusion protein that entails the use of an engineered, linker that covalently tethers Vif to EloB. This approach has successfully been used to express other proteins such as APOBEC3G and Vif. Vectors have been developed to facilitate the coexpression of Elongin C in the context of the desired fusion protein. The linker is further functionalized to include tandem His tags for affinity purification using NiNTA resin, as well as specific protease sites to remove the linker. A schematic representation of the linkers created and the proteins expressed is given in FIG. 2. Only the multiple cloning sites are provided since the vectors are commercially available. Each respective vector derived for production of EloB-Vif/EloC (i.e. the EloC-Vif fusion protein coexpressed with EloC protein) is described herein.

Vector pBVC-1:

Human Elongin B protein (amino acids 1-98) are expressed as an N-terminal fusion with HIV-1 (group M, subtype B) Vif protein (amino acids 102-173). Vif includes a C-terminal 6H is tag from multiple-cloning-site(MCS) 1 of pETDuet (Novagen, WI) for purification purposes. A designed 16 residue linker between ElonginB and Vif allows for flexibility and conformational changes upon binding ElonginC. The verified sequence of the 194 amino acid ElonginB-linker-Vif fusion protein is:

(SEQ ID NO: 1)
MGDVFLMIRRHKTTIFTDAKESSTVFELKRIVEGILKRPPDEQRLYK
DDQLLDDGKTLGECGFTSQTARPQAPATVGLAFRADDTFEALCIEPF
SSPPELGGGGTSGGGGSGGSLADQLIHLYYFDCFSDSAIRKALLGHI
VSPRCEYQAGHNKVGSLQYLALAALITPKKIKPPLPSVTKLTEDRGA
HHHHHH.

The flexible linker is underlined. This linker design contains neither the tandem His tag or protease cleavage sites. The fusion protein has a predicted MW of 21.1 kDa.

Human Elongin C protein (amino acids 17-112) is co-expressed from MCS2 of the pETDuet vector. Its entire sequence is:

(SEQ ID NO: 2)
MGYVKLISSDGHEFIVKREHALTSGTIKAMLSGPGQFAENETNEVNFRE
IPSHVLSKVCMYFTYKVRYTNSSTEIPEFPIAPEIALELLMAANFLDC.
The predicted MW is 10.9 kDa.

Vector pBVC-2:

Human Elongin B protein (amino acids 1-104) are expressed as a N-terminal fusion with HIV-1 (group M, subtype B) Vif protein (amino acids 102-173) from MCS1 of pETDuet. The 35 residue linker, ENLYFQSASGGHHHHGHHHHTSGGENLYFQSGGGS (SEQ ID NO: 3), between the Elongin B and Vif polypeptide termini comprises a HHHHGHHHH tag (SEQ ID NO: 4) for purification that is flanked by two tobacco etch virus (TEV) protease cleavage sites for linker removal. Human Elongin C protein (amino acids 17-112) are co-expressed from MCS2 of the pETDuet vector (sequence and calculated MW values are as indicated as described for pBVC-1). The entire amino acid sequence of this 212 residue ElonginB-linker-Vif fusion protein is:

(SEQ ID NO: 5)
MGDVFLMIRRHKTTIFTDAKESSTVFELKRIVEGILKRPPDEQRLYK
DDQLLDDGKTLGECGFTSQTARPQAPATVGLAFRADDTFEALCIEPF
SSPPELPDVMKENLYFQ/SASGGHHHHGHHHHTSGGENLYFQ/SGGG
SLADQLIHLYYFDCFSDSAIRKALLGHIVSPRCEYQAGHNKVGSLQY
LALAALITPKKIKPPLPSVTKLTEDR.

The two TEV protease cleavage-recognition sites are underlined in italics; cleavage occurs between Q and S of the underlined sequences. The internal tandem 4H is tag for NiNTA purification is shown in boldface type. The predicted MW of this ElonginB-linker-Vif peptide prior to TEV cleavage is 23.6 kDa. The calculated MW values of the Elongin B and Vif proteins after cleavage are 12.6 kDa and 8.4 kDa, respectively.

Vector pBVC-3:

Full-length human Elongin B protein (amino acids 1-118) are expressed as a N-terminal fusion with HIV-1 (group M, subtype B) Vif protein (amino acids 102-173) from MCS1 of pETDuet. The 35 residue linker, ENLYFQ/SASGGHHHHGHHHHTSGGENLYFQ/SGGGS (SEQ ID NO: 3), between Elongin B and Vif termini comprises a HHHHGHHHH (SEQ ID NO: 4) tag for purification that is flanked by two tobacco etch virus (TEV) protease cleavage sites for linker removal (comparable to pBVC-2). The main difference of this construct is the length of EloB, which was perceived to influence Cul5 binding. Human Elongin C protein (amino acids 17-112) are co-expressed from MCS2 of the pETDuet vector (sequence and MW values for EloC are as indicated above for pBVC-2). The entire sequence of this 226 residue ElonginB-linker-vif fusion protein is:

(SEQ ID NO: 6)
MGDVFLMIRRHKTTIFTDAKESSTVFELKRIVEGILKRPPDEQRLY
KDDQLLDDGKTLGECGFTSQTARPQAPATVGLAFRADDTFEALCIE
PFSSPPELPDVMKPQDSGSSANEQAVQENLYFQ/SASGGHHHHGHH
HHTSGGENLYFQ/SGGGSLADQLIHLYYFDCFSDSAIRKALLGHIV
SPRCEYQAGHNKVGSLQYLALAALITPKKIKPPLPSVTKLTEDR.

The two TEV protease cleavage-recognition sites are underlined in italics; cleavage occurs between Q and S of the underlined sequences. The predicted MW value of this ElonginB-linker-Vif peptide prior to TEV cleavage is 25.0 kDa. The predicted MW values of the Elongin B and Vif proteins after TEV cleavage are 14.0 kDa and 8.4 kDa, respectively.

Vector pBVC-4:

Full-length human Elongin B protein (amino acids 1-118) are expressed as a N-terminal fusion with HIV-1 (group M, subtype B) Vif protein (amino acids 95-173) from MCS1 of pETDuet. A 34 residue linker, ENLYFQ/SASGGHHHHGHHHHTSGGENLYFQ/SGGGS (SEQ ID NO: 3), between Elongin B and Vif termini comprises a HHHHGHHHH (SEQ ID NO: 4) tag for purification that is flanked by two tobacco etch virus (TEV) protease cleavage sites for linker removal. The main difference here to pBVC-3 is that the N-terminus of Vif has been extended to start at amino cid 95 rather than 102, which is hypothesized to influence Vif dimerization and/or Cul5 interactions. Human Elongin C protein (amino acids 17-112) is co-expressed from MCS2 of the pETDuet vector (sequence and MW are as indicated above for pBVC-3). The entire sequence of this 232 amino acid ElonginB-linker-Vif fusion protein is:

(SEQ ID NO: 7)
MGDVFLMIRRHKTTIFTDAKESSTVFELKRIVEGILKRPPDEQRLYK
DDQLLDDGKTLGECGFTSQTARPQAPATVGLAFRADDTFEALCIEPF
SSPPELPDVMKPQDSGSSANEQAVQENLYFQ/SASGGHHHHGHHHHT
SGGENLYFQ/SGGGSTQVDPELADQLIHLYYFDCFSDSAIRKALLGH
IVSPRCEYQAGHNKVGSLQYLALAALITPKKIKPPLPSVTKLTEDR.

The two TEV protease cleavage-recognition sites are underlined in italics; cleavage occurs between Q and S of the underlined sequences. The tandem 4H is tags are indicated in bold. The predicted MW of this ElonginB-linker-Vif fusion protein prior to TEV cleavage is 25.6 kDa. The predicted MW values of the Elongin B and Vif proteins after cleavage are 14.0 kDa and 8.4 kDa, respectively.

Vector pG-Cul5N:

Human Cullin5 (amino acids 2-384) was expressed as a truncated polypeptide with mutations V341R and L345D, which were introduced based on homology to the reported Cul1 structure and expression strategy. The two hydrophobic-to-hydrophilic mutations (V341R, L345D) are required to maintain solubility of the Cullin5 moiety when expressed as an N-terminal truncation. The C-terminally truncated Cul5 protein (i.e. Cul5N) is expressed as a C-terminal fusion with GST (glutathione S transferase) protein in MCS1 of pRSFDuet (Novagen, WI). A PreScission™ Protease cleavage site was introduced between the GST moleculae and the Cul5N protein. The sequence expressed from pG-Cul5N is:

(SEQ ID NO: 8)
MGSPILGYWKIKGLVQPTRLLLEYLEEKYEEHLYERDEGDKWRNKK
FELGLEFPNLPYYIDGDVKLTQSMAIIRYIADKHNMLGGCPKERAE
ISMLEGAVLDIRYGVSRIAYSKDFETLKVDFLSKLPEMLKMFEDRL
CHKTYLNGDHVTHPDFMLYDALDVVLYMDPMCLDAFPKLVCFKKRI
EAIPQIDKYLKSSKYIAWPLQGWQATFGGGDHPPKSDLEVLFQ/GP
MHMGGSTSNLLKNKGSLQFEDKWDFMRPIVLKLLRQESVTKQQWFD
LFSDVHAVCLWDDKGPAKIHQALKEDILEFIKQAQARVLSHQDDTA
LLKAYIVEWRKFFTQCDILPKPFCQLEITLMGKQGSNKKSNVEDSI
VRKLMLDTWNESIFSNIKNRLQDSAMKLVHAERLGEAFDSQLVIGV
RESYVNLCSNPEDKLQIYRDNFEKAYLDSTERFYRTQAPSYLQQNG
VQNYMKYADAKLKEEEKRALRYLETRRECNSVEALMECCVNALVTS
FKETILAECQGMIKRNETEKLHLMFSLMDKVPNGIEPMLKDLEEHI
ISAGLADMVAAAETITTDSEKYREQLDTLFNRFSKLVKEAFQDDPR
FLTARDKAYKAVVNDATIFK.

The PreScission™ Protease cleavage site is underlined; cleavage occurs between Q and G of the underlined sequence (shown above). The predicted MW of the GST-Cul5N protein is 71.6 kDa. After cleavage with PreScission™ Protease, the predicted MW of the Cul5N moiety is 45.1 kDa.

Expression and Purification of EloB(1-98)-linker-Vif(102-173)/(EloC(17-112):

BL21 (DE3) cells (Invitrogen, Carlsbad, Calif.) transformed with vector pBVC-1 were incubated in 50 mL LB media containing 100 ug/mL carbenicillin at 37° C. for 12 hr in 0.25 L flasks with shaking at 225 rpm. The cell culture was diluted to 0.05 OD₆₀₀ in LB containing 100 ug/mL carbenicillin and growth continued until the OD₆₀₀ was 0.6. The temperature was reduced to 30° C. and 1 mM IPTG was added for induction of expression. After a 4 hour incubation at 30° C., the cells were harvested by centrifugation at 4000 rpm for 15 min. Cell pellets were removed from the supernatant and flash frozen in liquid nitrogen prior to storage at −80° C.

Cell pellets were thawed and solubilized with 4 mL of Cell Lysis Buffer (comprising 400 mM NaCl, 50 mM HEPES pH 7.4, 5 mM beta-mercaptoethanol (B-Me), 10 mM imidazole) per 1 gram of cells. One tablet of EDTA-free protease inhibitor (Roche Applied Sciences, Indianapolis, Ind.) is added per 10 mL of cell suspension. Lysozyme was added to a final concentration of 2 mg/mL and the cell suspension was incubated on ice for 20 min, followed by sonication. 100 ug/mL RNase A (Sigma, St. Louis, Mo.) and 100 ug/mL DNase I (Sigma) were added to the cell suspension. After a 20 min incubation on ice the cell suspension was centrifuged at 13,000 rpm for 25 min. The supernatant was incubated with Nickel-nitrilo-triacetic acid (NiNTA) resin (Qiagen, Valencia, Calif.) at 4° C. for 2 hr. The resin was washed with 40 volumes of Buffer A (400 mM NaCl, 50 mM HEPES pH 7.4, 10 mM imidazole, 5 mM B-Me), followed by 10 volumes of Buffer B (Buffer A plus 50 mM imidazole). Protein was eluted with 5 volumes of Buffer C (Buffer A plus 240 mM imidazole). The NiNTA-purified complex is then passed through a size-exclusion chromatography (SEC) column of Sephacryl S-100 (GE Healthcare, Piscataway, N.J.) using a Beckman (Fullerton, Calif.) System Gold 126 HPLC pump equilibrated with Buffer D (125 mM NaCl, 25 mM HEPES pH 7.4, 5 mM B-mercaptoethanol). Protein elution was monitored with a Beckman System Gold 168 UV/Vis diode array spectrophotometer at 280 nm. The protein eluted with several peaks; the predominant peak eluted at a MW close to the predicted MW of a 1:1 EloB(1-98)-linker-Vif(102-173)/EloC(17-112) complex. Protein fractions corresponding to the predominant peak were pooled and subjected to a second round of SEC. Protein eluted from the column as a single peak with an apparent MW close to the predicted MW of a 1:1 EloB(1-98)-linker-Vif(102-173)/EloC(17-112) complex.

Expression and Purification of EloB(1-104)/EloC(17-112)/vif(102-173):

BL21 (DE3) cells (Invitrogen, Carlsbad, Calif.) transformed with vector pBVC-2 were incubated in 50 mL LB media+100 ug/mL carbenicillin at 37° C. for 12 hr in 0.25 L flasks shaking at 225 rpm. The cell culture was diluted to 0.05 OD₆₀₀ in LB containing 100 ug/mL carbenicillin and incubation continued until OD₆₀₀ was 0.6. The temperature was reduced to 30° C. and 1 mM IPTG was added. After a 4 hr induction at 30° C., cells were harvested by centrifugation at 4,000 rpm for 15 min. Cell pellets were removed from the supernatant and frozen in liquid nitrogen.

Cell pellets were thawed and solubilized with 4 ml of Cell Lysis Buffer per 1 gram of cells. One tablet of EDTA-free protease inhibitor (Roche Applied Sciences, Indianapolis, Ind.) is added per 10 mL of cell suspension. Lysozyme was added to a final concentration of 2 mg/mL and the cell suspension was incubated on ice for 20 min, followed by sonication. 100 ug/mL RNase A (Sigma, St. Louis, Mo.) and 100 ug/ml DNase I (Sigma) were added to the cell suspension. After a 20 min incubation on ice the cell suspension was centrifuged at 13,000 rpm for 25 mM. The supernatant was incubated with NiNTA resin (Qiagen, Valencia, Calif.) at 4° C. for 2 hr. The resin was washed with 40 volumes of Buffer A, followed by 10 volumes of Buffer B. Protein was eluted with 5 volumes of Buffer C. The eluted protein was buffer exchanged into Buffer A on a 10 DG disposable desalting column (BioRad, Hercules, Calif.) to remove imidazole. The linker between ElonginB and vif was removed by cleavage with 5 units of ProTEV protease (Promega, Madison Wis.) per mg of eluted protein at 8° C. for 24 hr. The cleaved linker, uncleaved protein complex, and partially cleaved protein complex were removed by incubation with NiNTA resin. The cleaved protein complex comprising EloB(1-104), EloC(17-112), and Vif (102-173) remains unbound and in solution. The protein complex is >95% pure at this point by SDS PAGE gels stained with Coomassie blue dye. The NiNTA-purified complex is then passed through an S-100 sephacryl column (GE Healthcare, Piscataway, N.J.) using a Beckman (Fullerton, Calif.) System Gold 126 HPLC pump equilibrated with Buffer D. Protein elution was monitored with a Beckman System Gold 168 UV/Vis diode array spectrophotometer at 280 nm. The protein eluted with a predominant peak at an apparent Mw close to the predicted Mw of a 1:1:1 EloB(1-104)/EloC(17-112)/Vif(102-173) complex.

Expression and Purification of EloB(1-118)/EloC(17-112)/vif(102-173):

BL21 (DE3) cells (Invitrogen, Carlsbad, Calif.) transformed with vector pBVC-3 were incubated in 50 mL LB media+100 ug/mL carbenicillin at 37° C. for 12 hr in 0.25 L flasks shaking at 225 rpm. The cell culture was diluted to 0.05 OD₆₀₀ in LB containing 100 ug/mL carbenicillin and incubation continued until OD₆₀₀ was 0.6. The temperature was reduced to 30° C. and 1 mM IPTG was added. After a 4 hr incubation at 30° C., the cells were harvested by centrifugation at 4,000 rpm for 15 min. Cell pellets were removed from the supernatant and frozen in liquid nitrogen.

Cell pellets were thawed and solubilized with 4 mL of Cell Lysis Buffer per 1 gram of cells. 1 tablet of EDTA-free protease inhibitor (Roche Applied Sciences, Indianapolis, Ind.) is added per 10 mL of cell suspension. Lysozyme was added to a final concentration of 2 mg/mL and the cell suspension was incubated on ice for 20 min, followed by sonication. 100 ug/mL RNase A (Sigma, St. Louis, Mo.) and 100 ug/mL DNase I (Sigma) were added to the cell suspension. After a 20 min incubation on ice the cell suspension was centrifuged at 13,000 rpm for 25 min. The supernatant was incubated with NiNTA resin (Qiagen, Valencia, Calif.) at 4° C. for 2 hr. The resin was washed with 40 volumes of Buffer A, followed by 10 volumes of Buffer B. Protein was eluted with 5 volumes of Buffer C (Buffer A plus 240 mM imidazole). The eluted protein was buffer exchanged into Buffer A on a 10 DG disposable desalting column (BioRad, Hercules, Calif.) to remove imidazole. The linker between EloB and Vif was removed by cleavage with 5 units of ProTEV protease (Promega, Madison Wis.) per mg of eluted protein at 8° C. for 24 hr. The cleaved linker, uncleaved protein complex, and partially cleaved protein complex were removed by incubation with NiNTA resin. The cleaved protein complex comprising EloB(1-118), EloC(17-112), and Vif (102-173) remains unbound and in solution. The protein complex is >95% pure at this point. The NiNTA-purified complex is then passed through a SEC S-100 Sephacryl column (GE Healthcare, Piscataway, N.J.) using a Beckman (Fullerton, Calif.) System Gold 126 HPLC pump equilibrated with Buffer D. Protein elution was monitored with a Beckman System Gold 168 UV/vis diode array spectrophotometer at 280 nm. The protein eluted with a predominant peak at MW close to the predicted MW of a 1:1:1 EloB(1-118)/EloC(17-112)/Vif(102-173) complex.

### Expression and Purification of EloB(1-118)/EloC(17-112)/Vif(95-173):

BL21 (DE3) cells (Invitrogen, Carlsbad, Calif.) transformed with vector pBVC-4 were incubated in 50 mL LB media+100 ug/mL carbenicillin at 37° C. for 12 hr in 0.25 mL flasks shaking at 225 rpm. The cell culture was diluted to 0.05 OD₆₀₀ in LB containing 100 ug/mL carbenicillin and incubation continued until OD₆₀₀ was 0.6. The temperature was reduced to 30° C. and 1 mM IPTG was added. After a 4 hr induction at 30° C., the cells were harvested by centrifugation at 4,000 rpm for 15 min. Cell pellets were removed from the supernatant and frozen in liquid nitrogen.

Cell pellets were thawed and solubilized with 4 mL of Cell Lysis Buffer per 1 gram of cells. One tablet of EDTA-free protease inhibitor (Roche Applied Sciences, Indianapolis, Ind.) was added per 10 mL of cell suspension. Lysozyme was added to a final concentration of 2 mg/mL and the cell suspension was incubated on ice for 20 min, followed by sonication. 100 ug/mL RNase A (Sigma, St. Louis, Mo.) and 100 ug/ml DNase I (Sigma) were added to the cell suspension. After a 20 min incubation on ice the cell suspension was centrifuged at 13,000 rpm for 25 min. The supernatant was incubated with NiNTA resin (Qiagen, Valencia, Calif.) at 4° C. for 2 hr. The resin was washed with 40 volumes of Buffer A, followed by 10 volumes of Buffer B. Protein was eluted with 5 volumes of Buffer C. The eluted protein was buffer exchanged into Buffer A on a 10 DG disposable desalting column (BioRad, Hercules, Calif.) to remove imidazole. The linker between EloB and Vif was removed by cleavage with 5 units of ProTEV protease (Promega, Madison Wis.) per mg of eluted protein at 8° C. for 24 hr. The cleaved linker, uncleaved protein complex, and partially cleaved protein complex were removed by incubation with NiNTA resin. The cleaved protein complex comprising EloB(1-118), EloC(17-112), and Vif (102-173) remains unbound and in solution. The protein complex is >95% pure at this point. The NiNTA-purified complex is then passed through an S-100 Sephacryl column (GE Healthcare, Piscataway, N.J.) using a Beckman (Fullerton, Calif.) System Gold 126 HPLC pump equilibrated with Buffer D. Protein elution was monitored with a Beckman System Gold 168 UV/Vis diode array spectrophotometer at 280 nm. The protein eluted with a predominant peak at a MW close to the predicted MW of a 1:1:1 EloB(1-118)/EloC(17-112)/Vif(95-173) complex.

### Expression and Purification of GST-Cul5(N)(2-384)(V341R, L345D):

BL21 (DE3) cells (Invitrogen) transformed with vector pG-Cul5N were incubated in 50 mL LB media containing 100 ug/mL carbenicillin at 37° C. for 12 hr in 0.25 L flasks shaking at 225 rpm. The cell culture was diluted to 0.05 OD₆₀₀ in LB containing 100 ug/mL carbenicillin and incubation continued until OD₆₀₀ was 0.6. The temperature was reduced to 30° C. and 1 mM IPTG was added. After a 4 hr induction at 30° C., the cells were harvested by centrifugation at 4,000 rpm for 15 min. Cell pellets were removed from the supernatant and frozen in liquid nitrogen.

Cell pellets were thawed and solubilized with 4 ml of Cell Lysis Buffer-2 (200 mM NaCl, 50 mM HEPES pH 7.4, 5 mM B-Me) per 1 gram of cells. One tablet of EDTA-free protease inhibitor (Roche Applied Sciences, Indianapolis, Ind.) was added per 10 mL of cell suspension. Lysozyme was added to a final concentration of 2 mg/mL and the cell suspension was incubated on ice for 20 min, followed by sonication. 100 ug/mL RNase A (Sigma, St. Louis, Mo.) and 100 ug/mL DNase I (Sigma) was added to the cell suspension. After a 20 min incubation on ice the cell suspension was centrifuged at 13,000 rpm for 25 min. The supernatant was incubated with glutathione resin (GE Healtcare) at 4° C. for 2 hr. The resin was washed with 40 volumes of Buffer E (200 mM NaCl, 50 mM HEPES pH 7.4, 5 mM B-Me). Protein was eluted with 5 volumes of Buffer F (Buffer E plus 25 mM glutathione). The glutathione-resin-purified complex is then passed through an S-100 Sephacryl column (GE Healthcare, Piscataway, N.J.) using a Beckman (Fullerton, Calif.) System Gold 126 HPLC pump equilibrated with Buffer D. Protein elution was monitored with a Beckman System Gold 168 UV/Vis diode array spectrophotometer at 280 nm. The protein eluted with a predominant peak at a MW close to the predicted MW of monomeric GST-Cul5(N).

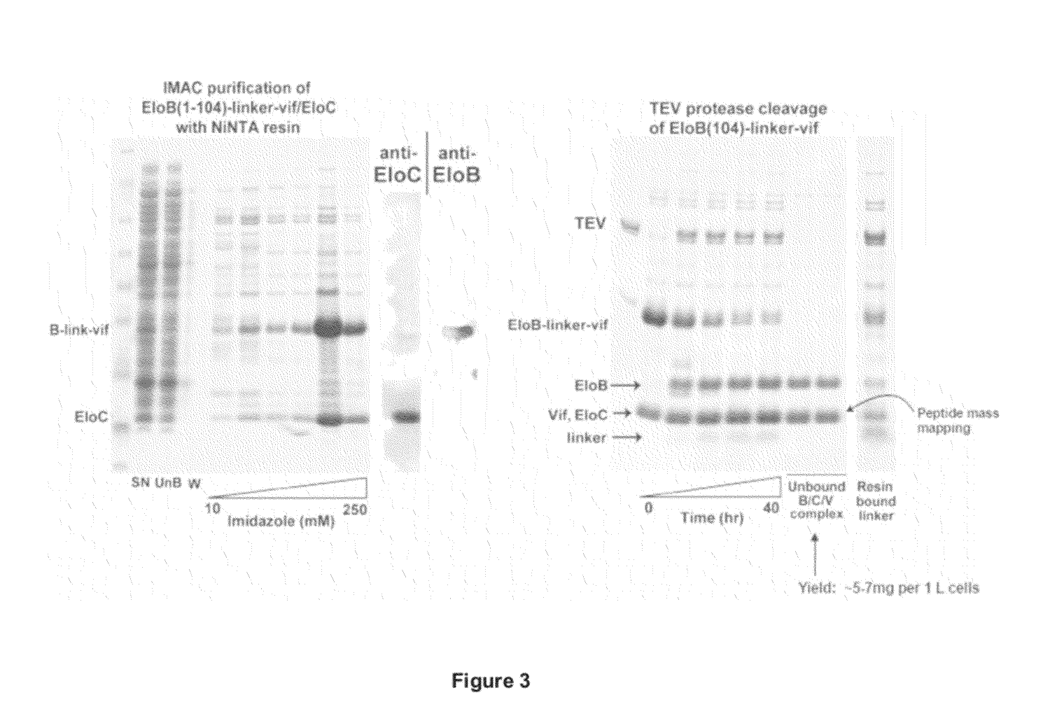

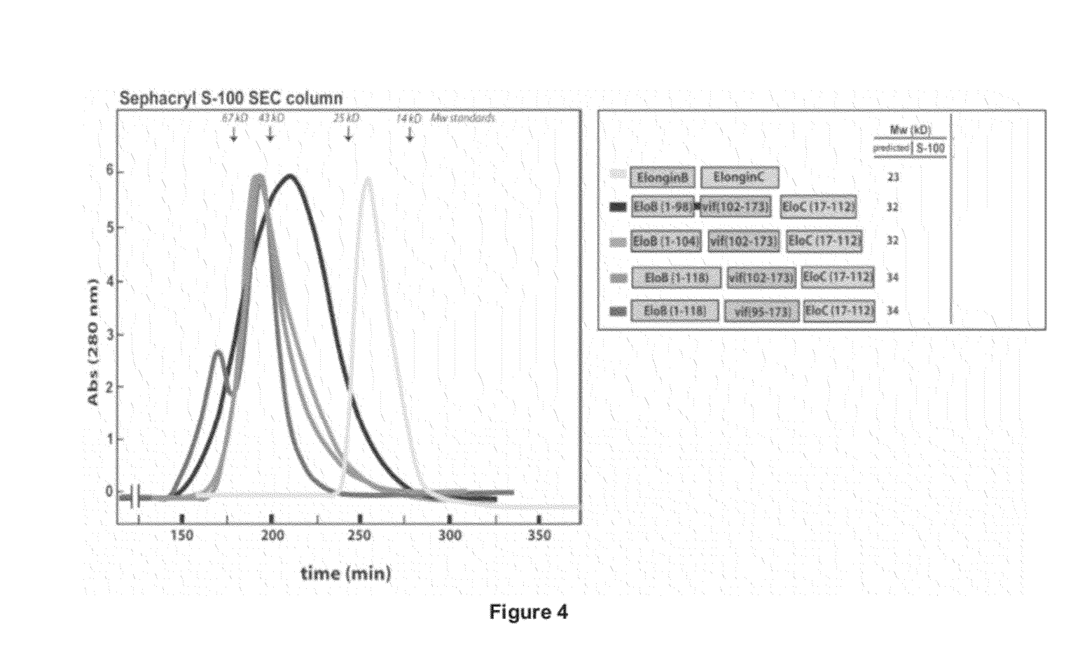

It was observed that the resulting tripartite complex remains folded as a compact globular shape in solution as assessed by gel filtration chromatography and dynamic light scattering (FIGS. 3 and 4).

### Example 2

### Vif-Mediated Interaction

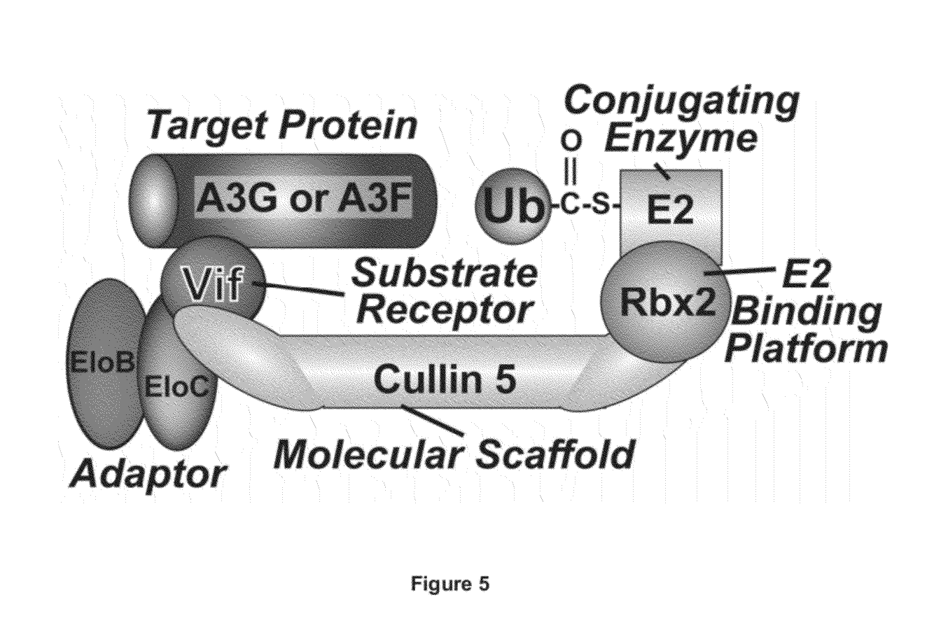

The next set of experiments was designed to develop an assay that demonstrates the biologically relevant assembly of purified components of Elongin B, Elongin C, Cullin 5 and Vif. The significance of the assembled complex is that it provides a model system to probe the HIV-1 Vif-mediated interaction between Cul5 and the EloB/C complex. To evade the host cell's immune system, HIV-1 utilizes the Vif protein as a substrate receptor that recruits the innate, antiviral factors APOBEC3G (A3G) and APOBEC3F (A3F) to the cell's own E3 ligase machinery (FIG. 5). Molecular interactions between the HIV-1 protein Vif and the human protein Cul5 represent a novel host-virus protein-protein interface that is necessary to promote viral infectivity. The ability to generate a soluble, pure complex comprising EloB/C/Vif/Cul5, and an assay to detect the interaction between Vif and Cul5 represents a major milestone in the field. This accomplishment opens the door to high throughput drug screening, as well as a means to probe the specific amino acid residues required for the molecular interaction between Vif and Cul5.

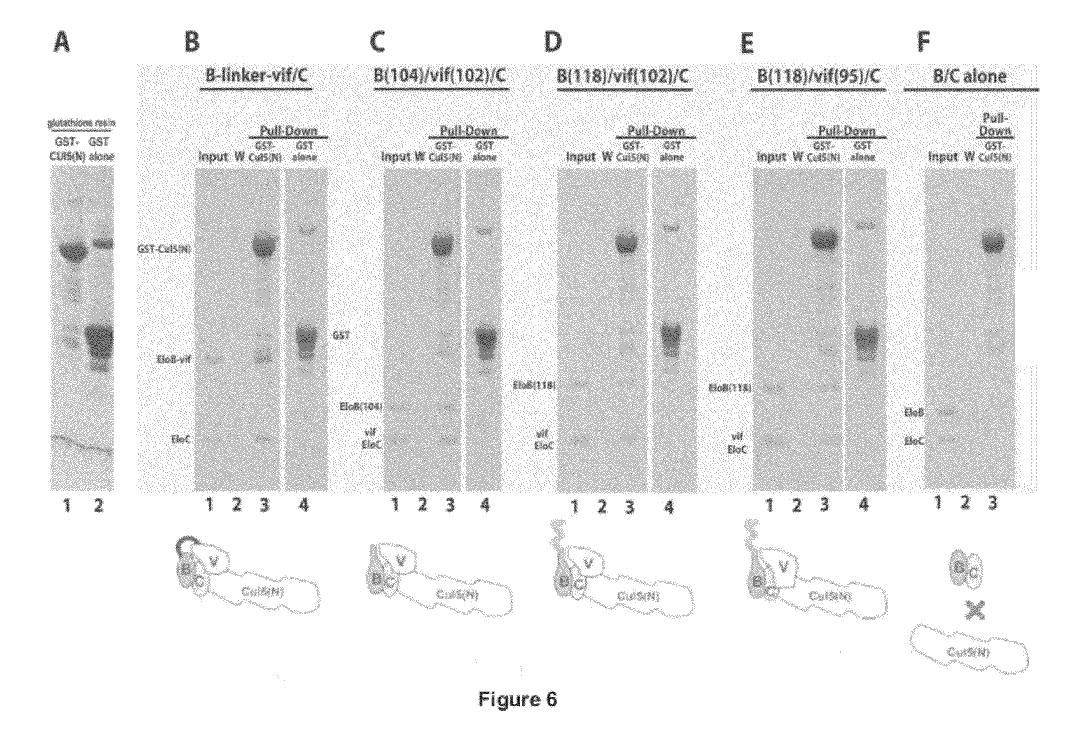

A non-limiting assay is a protein pulldown assay in which Cul5 is tagged with glutathione-S-transferase (GST). The GST-Cullin5 N-terminal protein is soluble and competent to recruit the tripartite ElonginB/ElonginC/Vif complex in the pulldown assay. Non-specific binding was not observed. Formation of the pulldown interaction is dependent on the presence of Vif (95-173), but has also been observed with shorter segments (102-173) (FIG. 6).

One mL of a 0.1 mg/mL purified GST-Cul5(N) or GST alone is incubated with 50 ul of packed glutathione sepharose 4B resin (GE Healthcare) for 2 hr at 4° C. GST-Cul5(N)-bound resin and GST-bound resin were washed extensively with 4 ml Buffer E (125 mM NaCl, 25 mM HEPES pH 7.4, 5 mM B-Me, 0.01% Brij-35). 10 ul of GST-Cul5(N)-bound resin and 10 ul of GST-bound resin were combined with 10 ul 2×SDS buffer, boiled for 4 min and examined by SDS-PAGE (FIG. 6A, lanes 1 & 2 for GST-Cul5(N)-bound resin and GST-bound resin, respectively). The remaining 40 ul of GST-Cul5(N)-bound resin and GST-bound resin were each incubated separately for 2 hr at 4° C. with 2.5 ml each of 0.1 mg/mL of one of the purified EloB/EloC/Vif complexes described above [i.e. EloB-Vif/EloC from pBVC-1 (FIG. 6B, lane 1); EloB(1-104)/EloC(17-112)/Vif(102-173) from pBVC-2 (FIG. 6C, Lane 1); EloB(1-118)/EloC(17-112)/Vif(102-173) from pBVC-3 (FIG. 6D, Lane 1); EloB(1-118)/EloC(17-112)/vif(95-173) (FIG. 6E, Lane 1); and EloB/EloC alone as a negative control (FIG. 6F). Subsequently, resins are spun down, the supernatant is removed, and the resin is again washed with 4×1 mL aliquots of Buffer E. The final wash aliquot was diluted 1:1 in 2×SDS buffer and examined by SDS-PAGE (Lane 2 of FIGS. 6B, 6C, 6D, 6E, and 6F). Resin was then mixed with 30 uL of 2×SDS-PAGE loading buffer, boiled for 4 min, and examined by SDS-PAGE (Lane 3 of FIGS. 6B, 6C, 6D, 6E, and 6F) for GST-Cul5(N) bound resin and associated EloB/Vif/EloC, and Lane 4 of FIGS. 6B, 6C, 6D, and 6E for GST (alone) bound to resin, which is not expected to interact with the EloB/Vif/EloC complex.

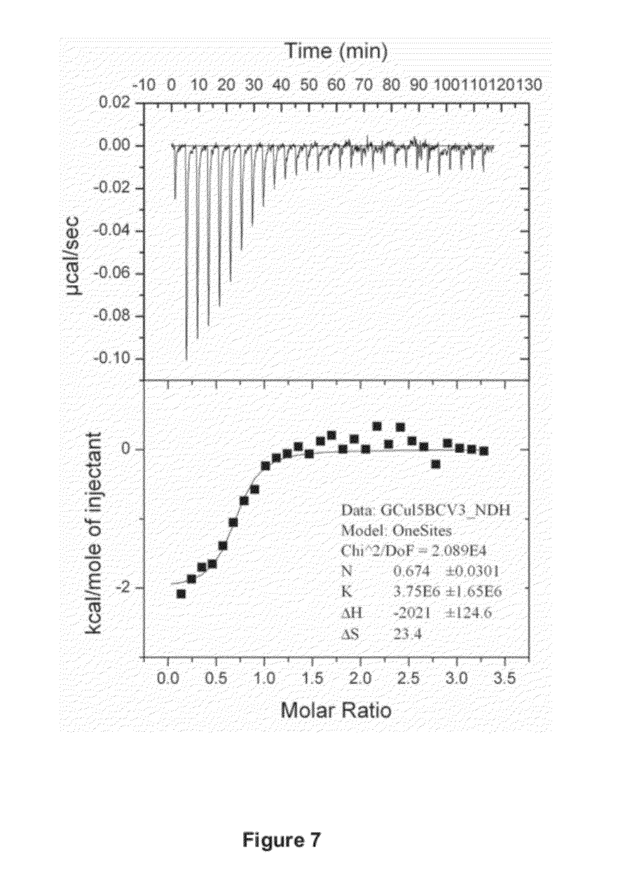

The next set of experiments was designed to characterize the specific binding between Cul5 and EloB/Vif/EloC. Isothermal Titration calorimetry (ITC) experiments were performed with a VP-ITC isothermal titration calorimeter (MicroCal, Northampton, Mass.) at 30° C. using a reference power of 15 ucal/sec and a 307 rpm stirring rate. Purified GST-Cul5(N) and EloB(1-104)/EloC(17-112)/Vif(102-173) were dialyzed against 1300 volumes of dialysis buffer (100 mM NaCl, 20 mM HEPES pH 7.4, 0.2 mM TCEP) at 4° C. for 6 hr. Thirty 10 uL aliquots of 150 uM GST-Cul5(N) were titrated into the 1.42 mL solution of 10 uM EloB(1-104)/EloC(17-112)/Vif(102-173) complex at 240 second intervals. The heat of binding was monitored as shown in FIG. 7. Data were fit using the MicroCal software package Origin 7.0. The heats of injection from the last three injections were averaged and subtracted from all data points to account for the heat of dilution upon titrating GST-Cul5(N) into dialysis buffer. The results revealed an equilibrium association constant K_{A} from the fit of the experimental data to a one-site-binding model is 3.75×10⁶ M⁻¹ (K_{D}=266 nM). Notably, the reported dissociation constants between complexes of cellular SOCS-box proteins with EloB/EloC and GST-Cul5(N) have ranged from <100 nM to 1000 nM; thus, this value is not unexpected, but represents the first such characterization of the Cul5 interaction with the HIV-1 protein. Importantly, this experiment verifies the binding interaction between the EloB/EloC/Vif complex and GST-Cul5(N), and corroborates the GST-Cul5(N) pull-down experiments. Importantly, the use of ITC requires large amounts of proteins and provides an experimental proof of principle for the robustness of our expression and purification methodology, which should be translatable to high-throughput screening. Knowledge of the thermodynamic parameters for Cul5 binding to EloB/Vif/EloC complexes are essential for the design of hearty high throughput screens since it reveals binding association from a highly quantitative vantage point (as compared to pulldown assays alone).

The screening assay of the invention provides an in vitro forum to identify small molecule compounds in a high-throughput format that selectively bind to Vif and/or inhibit Vif-dependent interactions between the human antiviral proteins APOBEC3G or APOBEC3F and the cellular ubiquitination machinery whose function is co-opted by the virus and whose independent and nonselective targeting would otherwise be toxic to the host cell. The key interaction targeted in the assay is between Cullin5 of the host Cullin-RING ligase complex and the C-terminal ‘Cullin-Box’ of Vif, which is included as one of the proteins in our EloginB/ElonginC/Vif tripartite complex. Specifically targeting this region of Vif avoids toxic effects to the cell that may arise from targeting the ubiquitination machinery of the host.

The results presented herein demonstrate a solution in the art for assaying for agents that bind to Vif as here-to-fore the methods for purifying sufficient soluble and functional Vif for high throughput screening were not known. The propensity of recombinant Vif to aggregate when expressed alone and during isolation and purification has render drug targeting of this essential viral protein impossible. Compounds that bind to Vif are candidate antiviral compounds as they may inhibit Vif dependent ubiquitination of APBOEC3G/3F but also may interfere with Vif binding to APOBEC3G/3F or Vif binding to viral capsid proteins and thereby enable increase amounts of APOBEC3G/3F to become incorporated into viral particles leading to the inhibition of viral replication.

The results presented herein demonstrate a solution in the art for assaying for agents that interfere with the binding between Vif and Cul5. The main solution is a method to produce HIV-1 Vif in quantities sufficient for in vitro studies. The results led to the discovery of a method to produce an ElonginB/ElonginC/Vif complex, as well as a region of Cullin5 competent to bind Vif in the context of the former tripartite assembly. The biological significance is that resulting proteins form an interaction network that is necessary and sufficient for probing the Vif-mediated interaction between Cullin5 and ElonginB/C, which is essential to activate the Cullin-RING E3 ligase responsible for degradation of the innate antiviral proteins APOBEC3G and APOBEC3F. The mode of generating these proteins, their demonstrated solubility, and milligram production scale are well suited to development of an assay to screen for small molecule compounds that block the Vif interaction with Cullin5, thus preserving innate antiviral function. The approach disclosed here gets around a standing roadblock in the field in which traditional methods to produce Vif have revealed it is prone to aggregation or insolubility and therefore does not lend itself to in vitro assays required for large-scale, high-throughput screening.

Protein-protein interactions of vif are critical for its function as substrate receptor in the E3 ligase complex and HIV-1 infectivity. Thus, blocking one or more of these interactions could abrogate vif-mediated degradation of hA3G, leaving hA3G free to carry out its antiretroviral activities. Structure Assisted Drug Design (SADD) involves using pharmacophore maps for screening virtual compound libraries to identify compounds that may prevent vif protein-protein interactions (Waszkowycz et al., 2001, IBM Systems Journal 40:360-376).

A precedence for using SADD to develop small molecule inhibitors exists and is exemplified well with HIV-1 protease, for which a number of peptidomimetics, such as saquinavir, ritonavir, and indinavir have been designed and act as transition state analogs that bind and block HIV protease activity (Wlodawer, and Vondrasek, 1998, Annu Rev Biophys Biomol Struct 27:249-84). Many other transition state analogs for HIV-1 protease, as well as reverse transcriptase, have been developed with SADD and are in clinical use to treat HIV-1 as a part of HAART (highly active antiretroviral therapy) (Wlodawer, and Vondrasek, 1998, Annu Rev Biophys Biomol Struct 27:249-84).

### Example 3

### FqRET Assay for High-Throughput Screening

The next set of experiments was designed to develop an assay to detect the interaction between Cul5 and Vif in a high-throughput format. The significance of this assay is that it allows large-scale screening of compound libraries in an in vitro format. This assay allows for the identification of candidate molecules to block the Cul5 interaction with Vif. Since the assay relies on a Vif-mediated interaction with Cul5, the assay allows for selective targeting of the unique interface to obstruct Vif-mediated degradation of A3G or A3F.

The next set of experiments was designed to develop a Förster quenched resonance energy transfer (FqRET) assay for high-throughput screening of inhibitors of Vif-mediated binding of Cullin5 to the Elongin B/C complex. Without wishing to be bound by any particular theory, it is believed that since this process requires fusion with proteins such as EGFP and REACh2, the desired tag can be substituted during the cloning of the fusion proteins. For example, GST may be replaced with the 26.9 kDa EGFP protein at the N-terminus of Cullin5 without concern that it will interfere with Vif binding since the pulldown experiments already proved GST-Cullin5 was a competent target. With respect to the REACh2 (a variant of YFP) tag, it can be attached to either the N- or C-terminus of ElonginC, or to the C-terminus of Vif.

FqRET complexes can also be produced by chemically conjugating Cullin 5 in vitro with commercially available fluorescence donors and conjugating Elongin B-Vif in vitro with commercially available fluorescence quenchers. FqRET assays based on complexes composed of proteins chemically conjugated with donor and quencher have greater differentials between quenched and unquenched signals compared to what can be observed in complexes containing EGFP and REACh2 fusion proteins because chemical conjugation position multiple donors and quenchers along the coupled protein based on the availability of primary and secondary amine groups.

To the establishment a robust Förster quenched resonance energy transfer (FqRET) assay for high-throughput compound library screening in microtiter plates. The assay is based on selective placement of chromoproteins or chromophores that allow reporting on complex formation between the EloB/C/Vif protein and EloC in vitro. For example, an appropriately positioned FRET donor and FRET quencher results in a “dark” signal when the quaternary complex is formed. The assay is designed to achieve FqRET based on formation of a complex between EloB/Vif/EloC and Cul5 in which the latter is expressed as (StrepII)-EGFP-Cul5 fusion protein as the fluorescence donor, and the former is expressed as an EloB-Vif-ReACH2-6H is fusion protein, which is the fluorescence quencher. Quenched complexes assembled in vitro and dispensed into microtiter plates will retain low levels of fluorescence. When the appropriate compound has been identified to block the Vif-Cul5 interaction, the complex disassociates leading to a fluorescence signal. Without wishing to be bound by any particular theory, it is believed that the use of small molecule chromophores to tag the respective proteins as well, which should elicit the same response FqRET response.

Based on the disclosure presented herein, the design of an FqRET assay for high-throughput screening of inhibitors of Vif-mediated binding of Cullin 5 to the Elongin B/C complex can be adapted to any appropriate chemical conjugation of fluorescence donors and quenchers to interacting proteins involved in complex formation.

### Example 4

### Crystallization and X-Ray Diffraction

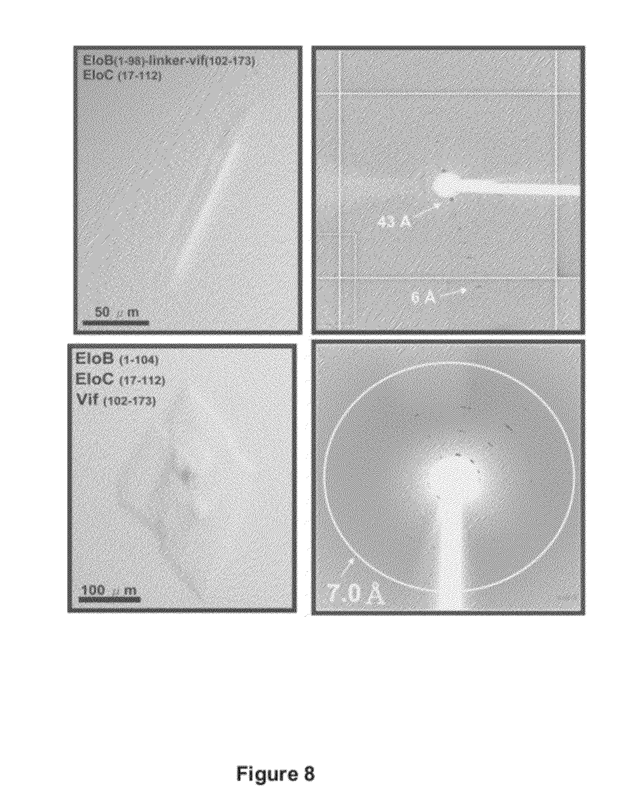

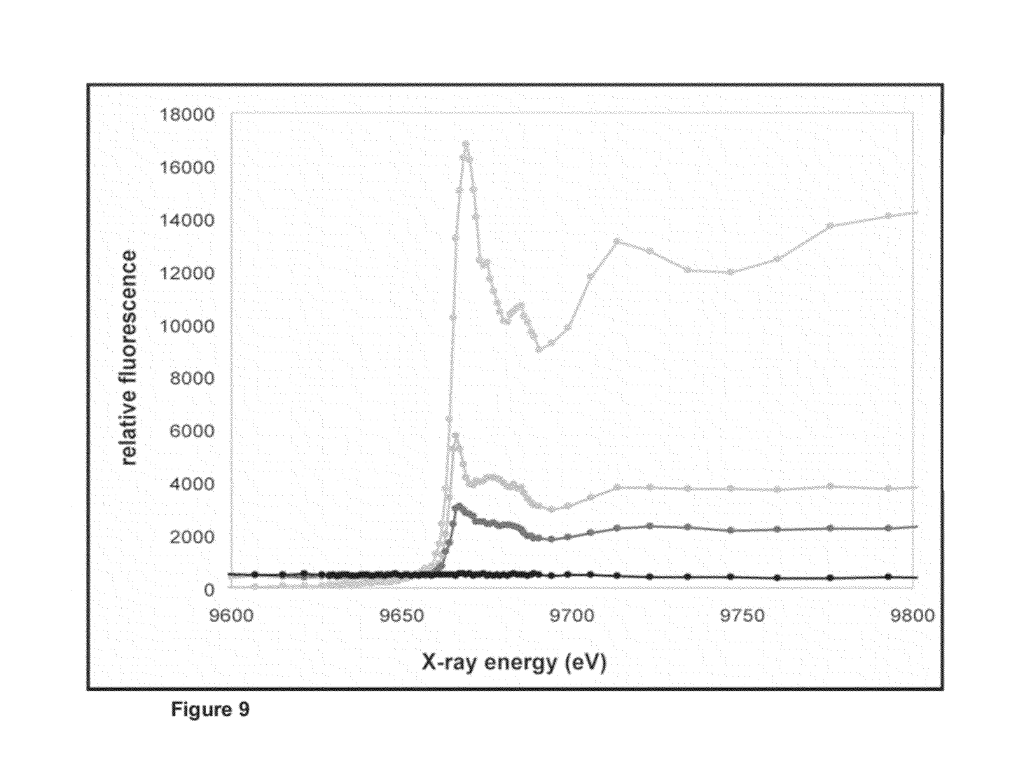

Several small crystals of EloB/Vif/EloC complexes have been grown that display Bragg diffraction upon exposure to x-rays. Purified EloB/Vif/EloC complexes have been screened for crystallization conditions between 5 and 45 mg/ml at 4° C. and 20° C. using the hanging-drop vapor diffusion method. Crystals of EloB(1-104)/EloC(17-112)/vif(102-173) complex grew from 0.1 M Tris-HCl, 20% 2-methyl-2,4-pentanediol (MPD) in three weeks at 20° C. Crystals of EloB(1-98)-linker-vif(102-173)/EloC(17-112) grew from 0.1 M Bicine, 20% MPD in 4 weeks at 20° C. Crystals were cryoprotected by serial transfer into synthetic mother liquor with 20, 25, 30, 35, 40% (v/v) MPD, mounted in rayon loops, and flash-frozen in liquid nitrogen. X-ray diffraction data was collected at the Stanford Synchrotron Radiation Laboratory (Stanford, Calif.). (FIG. 8).

The next set of experiments was designed to further characterize the EloB/Vif/EloC complexes. A fluorescence scan was performed on crystals of EloB(1-104)/EloC)/vif(102-173), EloB-link-vif/EloC, and small molecules with and without zinc. The increase in fluorescence at the zinc K absorbance edge (9.658 keV) supports the presence of zinc in the protein crystals. This suggests that the HCCH motif of vif coordinates zinc.

The results presented herein demonstrate the ability to produce highly purified (>95%) EloB/EloC/vif(C-term) complexes with a novel expression method. Crystallization trials were conducted for four different truncations of the EloB/EloC/vif complex; each complex differed by either length of EloB or vif or by presence of linker between EloB and vif; the length of EloC expressed (17-112) remained invariant. While each complex was capable of binding GST-Cul5(N), crystals grew for only the two smallest complexes (EloB(1-104)/EloC/vif(102-173) and EloB-linked-vif/EloC). Crystals grew in thin 2-D plates and rods. Although the diffraction was weak and the spots were streaky, the observed low resolution diffraction and the regular spacing between diffraction spots is supportive of macromolecular crystallization. Preliminary ITC experiments verified the binding observed between EloB(1-104)/EloC/vif(102-173) and GST-Cul5(N).

Experiments can be designed to focus on improving the quality of crystals to allow structure determination. Crystallization of the HCCH motif in vif may require the presence of Cul5. ITC experiments and GST pull-down experiments can be conducted to determine a minimal domain of Cul5 capable of forming a stable complex with EloB/Vif/EloC. Analytical ultracentrifugation sedimentation velocity experiments can be performed to determine the oligomeric state of purified B/C/vif complexes. These experiments can help guide crystallization experiments by identifying complexes most suitable for screening.

### Example 5

### Design of Recombinant Elongin B, ElonginC, Cullin 5 and Vif Proteins

The following experiments were designed to generate desired recombinant proteins. The term EloBCvif refers to a generalized ternary complex of human EloB, EloC, and HIV-1 vif from strain HXB2. In this context, the sequence lengths of the respective polypetides are unspecified. As discussed elsewhere herein, B refers to the full-length EloB protein (residues 1-118). B₁₀₄ refers to the EloB C-terminal truncation constructs (residues 1-104). C refers to the only EloC sequence used herein, comprising residues 17-112. The sequence length of vif is indicated in subscript form. Vif double mutations A123S/L124S, A123G/L124A, and A123V/L124F were introduced into BCvif₉₅₋₁₉₂ and are referred to as BCvif_{SS}, BCvif_{GA}, BCvif_{VF}. Additionally the double mutation, A123S/L124S, was introduced into BCvif₉₅₋₁₅₅ and is referred to as BCvif_{155-SS}.

The term B₉₈-LINKER-vif/C refers to a complex in which the fusion protein comprising EloB (residues 1-98) is covalently linked to vif (residues 102-173) with an intact GGGGTSGGGGSGGS (SEQ ID NO: 15) polypeptide linker.

### Design of an EloB-vif Linked Fusion Protein for Production of an EloB-vif/EloC Complex

An E. coli expression-optimized synthetic gene encoding human EloB₁₋₉₈-GGGGTSGGGGSGGS-HIV-1 HXB-2 vif₁₀₂₋₁₇₃-GAHHHHHH was generated by BioBasic, Inc. and cloned into MCS1 of pETDuet-1 (EMD4Biosciences) using the restriction enzymes NcoI and EcoRI at the 5′ and 3′ ends respectively. The linker sequence (GGGGTSGGGGSGGS; SEQ ID NO: 15) between EloB and vif was engineered to be flexible with a length to accommodate the spatial constraints imposed by the termini of the EloB-EloC-vif₁₄₀₋₁₅₅ model (PDB entry 3DCG){Stanley, 2008 #2360}. The DNA sequence of the linker contains SacI, SpeI, and BamHI restriction sites to facilitate modification of EloB and vif sequence lengths as well as the linker composition, as described elsewhere herein. A tagless human EloC₁₇₋₁₁₂ DNA sequence was purchased from Open Biosystems, and was PCR-amplified for cloning into MCS2 of the pETDuet-1 vector using NdeI and BglII. The complete dual expression vector is called pB₉₈-vif/C.

Dideoxynucleotide sequencing of this and all other pETDuet-1 vectors herein was performed with Novagen sequencing primers, pET Upstream and DuetDOWN1 for MCS1, and DuetUP2 and T7-terminator for MCS2. All sequence reactions were analyzed by the Functional Genomics Center (University of Rochester, N.Y.).

### Design of the EloB-vif Cleavable Fusion Proteins for Production of the EloB-EloC-vif Ternary Complexes.

The ternary complexes, B₁₀₄-C-vif₁₀₂₋₁₇₃, B₁₀₄C-vif₉₅₋₁₇₃, BC-vif₉₅₋₁₉₂, BC-vif₉₅₋₁₇₃, BC-vif₉₅₋₁₆₀, BC-vif₉₅₋₁₅₅, where B is full-length EloB₁₋₁₁₈ and C is EloC₁₇₋₁₁₂, were expressed from vectors described elsewhere herein that contain modifications in MCS1 of the parent vector pB₉₈-vif/C; expression of EloC from MCS2 was not altered. A synthetic dsDNA oligo (IDT) coding for LINK_{clv}, the 35-amino acid removable linker of sequence ENLYFQSASGGHHHHGHHHHTSGGENLYFQSGGGS (SEQ ID NO: 3, was cloned into MCS1 of pB₉₈-vif/C, replacing the previous non-removable linker. Two tobacco-etch virus (TEV) protease cleavage sites (ENLYFQ/S) flank the internal His-tag (HHHHGHHHH; SEQ ID NO: 4) and facilitate linker removal following protein purification. Likewise, synthetic DNA oligos were also used to increase the length of EloB₁₋₉₈ to either EloB₁₋₁₀₄ or full-length EloB₁₋₁₁₈. Expression sequences for the respective Vif₉₅₋₁₉₂, Vif₉₅₋₁₇₃, vif₉₅₋₁₆₀, and vif₉₅₋₁₅₅ sequences were PCR-amplified from HIV-1 (HXB-2) DNA and cloned into pB₉₈-vif/C, replacing the previous vif₁₀₂₋₁₇₃-GAHHHHHH sequence and removing the C-terminal His-tag. DNA and protein sequences of each dual expression pETDuet vector are provided in Table 1.

TABLE 1
Protein complex naming convention

| pETDuet-1 | Protein Complex | Proteins |
|---|---|---|
| Expression Vector | Name | (after TEV protease cleavage) |
| pB₉₈-vif/C | B₉₈-LINKER-vif/C | EloB (1-98)-LINKER-vif (102-173) |
| | | EloC (17-112) |
| pB₁₀₄-LINK_{CLV}-vif₁₀₂₋₁₇₃/C | B₁₀₄Cvif₁₀₂₋₁₇₃ | EloB (1-104) |
| | | EloC (17-112) |
| | | Vif (102-173) |
| pB₁₀₄-LINK_{clv}-vif₉₅₋₁₇₃/C | B₁₀₄Cvif₉₅₋₁₇₃ | EloB (1-104) |
| | | EIOC (17-112) |
| | | Vif (95-173) |
| pB-LINK_{clv}-vif₉₅₋₁₉₂/C | BCvif₉₅₋₁₉₂ | EloB (1-118) |
| | | EloC (17-112) |
| | | Vif (95-192) |
| pB-LINK_{clv}-vif₉₅₋₁₇₃/C | BCvif₉₅₋₁₇₃ | EloB (1-118) |
| | | EloC (17-112) |
| | | Vif (95-173) |
| pB-LINK_{clv}-vif₉₅₋₁₆₀/C | BCvif₉₅₋₁₆₀ | EloB (1-118) |
| | | EloC (17-112) |
| | | Vif (95-160) |
| pB-LINK_{clv}-vif₉₅₋₁₅₅/C | BCvif₉₅₋₁₅₅ | EloB (1-118) |
| | | EloC (17-112) |
| | | Vif (95-155) |
| pB-LINK_{clv}-vif_{AL/GA}/C | BCvif_{GA} | EloB (1-118) |
| | | EloC (17-112) |
| | | vif (95-192) (A123G, L124A) |
| pB-LINK_{clv}-vif_{AL/VF}/C | BCvif_{VF} | EloB (1-118) |
| | | EloC (17-112) |
| | | vif (95-192) (A123V, L124F) |
| pB-LINK_{clv}-vif_{AL/SS}/C | BCvif_{SS} | EloB (1-118) |
| | | EloC (17-112) |
| | | vif (95-192) (A123S, L124S) |
| pB-HIS/C | BC | EloB (1-118) |
| | | EloC (17-112) |
| pB-LINK_{clv}-vif(155)_{AL/SS}/C | BCvif_{155-SS} | EloB (1-118) |
| | | EloC (17-112) |
| | | Vif (95-155) (A123S, L124S) |
| pGST-Cul5(N) | GST-Cul5(N) | Cul5(2-384) (V341R, L345D) |
| | | GST |

### Site-Directed Mutagenesis and Expression of Vif Complexes and the EloB/C Heterodimer.

The vif double mutants A123G/L124A, A123V/L124F, A123S/L124S were each cloned in the context of the BC-vif₉₅₋₁₉₂ ternary complex by creating mutations in pB-LINK_{clv}-vif₉₅₋₁₉₂/C using the Quick Change Lightning Site-Directed Mutagenesis Kit (Stratagene) according to the manufacturer's protocol. Additionally, the A123S/L124S double mutant was produced in the context of the BC-vif₉₅₋₁₅₅ ternary complex by mutating pB-LINK_{clv}-vif₉₅₋₁₅₅/C. Similarly, the construct pB-His/C for expression of EloB₁₋₁₁₈-EloC₁₇₋₁₁₂ heterodimer was produced by site-directed mutagenesis of pB-LINK_{clv}-vif₉₅₋₁₉₂/C to incorporate a STOP codon immediately following the HHHHGHHHH (SEQ ID NO: 4) sequence, allowing for removal of the purification tag from EloB with the proximal TEV protease site. The mutant vectors, associated ternary BC-vif, and heterodimeric EloBC complexes are disclosed elsewhere herein.

### Expression of Soluble N-Terminal Cul5 as a GST-Fusion Protein.

To express GST-Cul5₂₋₃₈₄—hereafter called GST-Cul5(N)—human Cul5₂₋₃₈₄ DNA sequence (Open Biosystems) was amplified with primers comprising 5′ BamHI and 3′ EcoRI restriction enzyme sites and ligated into MCS1 of pRSFDuet-1 (EMD4Biosciencs). Subsequently, the DNA sequence of glutathione-S-transferase (GST) with a C-terminal linker containing a 3C protease site was PCR-amplified from pGEX-6 μl (GE Healthcare), which was cloned upstream of the Cul5 sequence using 5′ NcoI and 3′ BamHI restriction enzyme sites. To enhance solubility, the mutations V341R and L345D were introduced into Cul5 using site-directed mutagenesis (as described above). These two mutations are analogous to those reported for Cul1 (Zheng et al., 2002 Nature 416, 703-709). This vector—hereafter known as pGST-Cul5(N)—was sequenced with Novagen sequencing primers, ACYCDuetUP1 and DuetDOWN1.

### Expression of BCvif and EloBC Protein Complexes

A general description of expression of the tripartite complexes and Cul5(N) proteins is provided below. A single colony of BL21(DE3) (Invitrogen) transformed with one of the vectors listed in Table 1 was incubated in 50 ml of LB media containing an appropriate antibiotic—either 100 μg ml⁻¹ carbenicillin (pETDuet-1 vectors) or 60 μg ml⁻¹ kanamycin (pRSFDuet-1 vector)—at 37° C. for 10-12 h in a 0.25 L flask shaking at 225 rpm. The cell culture was diluted to 0.05 OD₆₀₀ in freshly prepared LB with the appropriate antibiotic and incubation continued until an OD₆₀₀ reading of ˜0.6. The temperature was reduced to 30° C. and 1 mM isopropyl-β-D-thiogalactosce (IPTG) was added. After 4 h incubation at 30° C., the cells were harvested by centrifugation at 2.8K×g for 15 min; cell pellets were frozen in liquid nitrogen and stored at −90° C.

### Purification of BCvif and EloBC Complexes

Cell pellets were thawed and resuspended with Cell Lysis Buffer (CLB) comprising 0.40 M NaCl, 0.050 M HEPES pH 7.4, 0.005 M (3-mercaptoethanol (β-Me) and 0.010 M imidazole; each gram of cells was resuspended with 4 ml. One tablet of EDTA-free protease inhibitor (Roche Applied Sciences) was added per 10 ml of cell lysis buffer. Lysozyme was added to a final concentration of 2 mg ml⁻¹ and the cell suspension was incubated on ice for 20 min prior to cell lysis with a Sonic Dismembrator 60 (Fisher Scientific). Nucleic acids were then digested by use of 50 μg ml⁻¹ RNase A (Sigma) and 50 μml⁻¹ DNase I (Sigma), which were added to the cell suspension. After 20 mM incubation on ice the suspension was centrifuged at 20K×g for 25 min. The supernatant was incubated with 1 ml nickel-nitrilo-triacetic acid (Ni-NTA) resin (Qiagen) per 4 g of cell pellet at 4° C. for 2 h. Protein-bound resin was washed with 40 bed volumes of Buffer A (0.40 M NaCl, 0.050 M HEPES pH 7.4, 0.010 M imidazole, 0.005 M B-Me), followed by ten bed volumes of Buffer B (Buffer A plus 0.040 M imidazole). Protein was eluted with Buffer C (Buffer A plus 0.240 M imidazole). The eluted protein was exchanged into Buffer A on a 10 DG disposable desalting column (BioRad) to reduce imidazole. The linker between EloB and vif (or the HIS tag of the BC dimer) was removed by protease cleavage with 5 units of ProTEV (Promega) per mg of eluted protein at 4°-8° C. for 24 h. The cleaved linker, uncleaved protein complex, and partially cleaved protein complexes were removed by incubation with Ni-NTA resin. The fully cleaved protein complex comprised individual EloB, EloC, and vif moieties did not bind and remained in solution with a purity level>95%. The Ni-NTA-treated complexes were subjected to a Sephacryl S-100 column (GE Healthcare) using a Beckman System Gold 126 HPLC pump equilibrated with Buffer D (0.125 M NaCl, 0.025 M HEPES pH=7.4, and 0.005 M β-mercaptoethanol). Protein elution was monitored with a Beckman System Gold 168 UV/vis diode array spectrophotometer at 280 nm. Protein fractions were collected with a Beckman SC 100 fraction collector. Appropriate fractions were pooled, concentrated to ˜2.5 mg ml⁻¹, frozen as beads in N₂(l), and stored at −90° C. Purification of the B₉₈-linked-vif/C was the same as described above except that it was not treated with ProTEV.

### Purification of Cul5(N)

Cell pellets were thawed and solubilized with CLB made in the absence of imidazole. Cells were lysed and nucleic acids were digested as described above. The supernatant was incubated with 1 ml packed Glutathione Sepharose 4B (GE Healthcare) per 4 g of cell pellet at 4° C. for 2 h. Protein-bound resin was washed with 40 bed volumes of CLB. The resin was then suspended in a volume of CLB equal to 4 bed volumes of the resin. On-resin cleavage was conducted using 10 units PreScission Protease (GE Healthcare) per ml resin at 4° C. for 48 h. This step efficiently liberates Cul5(N) by cutting the 3C protease site of fusion protein, which leaves GST immobilized on the resin. The desired protein was recovered by washing of the resin with four column volumes of CLB. The protein was concentrated to 5 mg ml⁻¹ and frozen as described elsewhere herein.

### Protein Identification

All proteins (Cul5, EloB, EloC, vif) were identified by peptide mass mapping of excised bands from coomassie-stained SDS-PAGE using an AB 4700 Proteomics Analyzer (PAN Facility, Stanford University) (data not shown).

### Dynamic Light Scattering Measurements of EloBCvif Complexes

DLS measurements were made on purified EloBCvif complexes to assess the size, aggregation, dispersity, and the likelihood of crystallization. Scattering data were collected from EloBC-vif complexes on a DynaPro 801 Molecular Sizing Instrument (Protein Solutions, Inc.) in a 12 μL cell with a 25 mW laser at 750 nm and 22° C. Protein concentrations ranged from 1.4 to 3.0 mg ml⁻¹. The hydrodynamic radius (Hᵣ) and apparent molecular weight of each sample was calculated using the software package Dynamics v. 4.0 (Protein Solutions, Inc.).

GST-Cul5 Pull-Down Experiments with EloB-EloC-Vif Complexes

A 1 ml volume of 0.1 mg ml⁻¹ purified GST-Cul5(N) or GST alone was incubated with 50 μl of packed Glutathione Sepharose 4B resin (GE Healthcare) for 2 h at 4° C. GST-Cul5(N)-bound resin and GST-bound resin were washed extensively with 4 ml Buffer E comprising 0.125 M NaCl, 0.025M HEPES pH 7.4, 0.005 M n-Me, 0.01% (v/v) Brij-35. Bound material was assessed by combining 10 μl of either GST-Cul5(N)-bound resin or 10 μl of GST-bound resin with 10 μl of 2×LDS buffer (Thermo Fisher Scientific), boiled for 4 min and examined by SDS-PAGE. The remaining 40 μl of GST-Cul5(N)-bound resin and GST-bound resin were each incubated separately for 2 h at 4° C. with 2.5 ml of 0.1 mg ml⁻¹ of the respective, purified protein complexes: B₉₈-linker-vif₁₀₂₋₁₇₃/C, B₁₀₄Cvif₁₀₂₋₁₇₃, BCvif₁₀₂₋₁₇₃, BCvif₉₅₋₁₇₃ or BC. Resins were subsequently spun down by microcentrifugation, the supernatant was removed, and the resin was again washed with four successive 1 ml aliquots of Buffer X. The final wash aliquot was diluted with an equal volume of 2×LDS buffer and examined by SDS-PAGE. The remaining 30 μl of resin was then mixed with 30 μl of 2×LDS buffer, boiled for 4 min, and separated by SDS-PAGE.

### Isothermal Titration Calorimetry

ITC experiments were conducted with a VP-ITC isothermal titration calorimeter (MicroCal) at 30° C. using a reference power of 15 μcal s⁻¹ and a 307 rpm stirring rate. Purified Cul5(N) and EloBC-vif complexes were dialyzed at 4° C. for 8 h against 1300 volumes of ITC buffer comprising 0.125 M NaCl, 0.20 M HEPES pH 7.4, 0.002 M TCEP. Twenty-nine 10 μl aliquots of Cul5(N), ranging in concentration from 150 to 190 uM were titrated at 240 s intervals into a cell harboring EloBC or EloBCvif complexes, ranging in concentration from 19 to 27 uM. For the titration of GST-Cul5(N) into BC, the concentrations were 90 and 11 uM, respectively. The specific concentration of protein in the syringe and cell for individual experiments is provided elsewhere herein. To account for the heat of dilution of the injectant (Cul5(N)) in most experiments, the heats of injection from the last three injections were averaged and subtracted from each data point. For Cul5(N) titration into BC, the heat of dilution of Cul5(N) was accounted for by subtraction of the value of heat equal to the value of the y-intercept of a line fit to the plot of the integrated heats of injection versus injection number from the titration of Cul5(N) into buffer alone. Injection of protein into pure buffer was also conducted to establish that the heat of dilution was relatively constant over the course of the entire experiment.

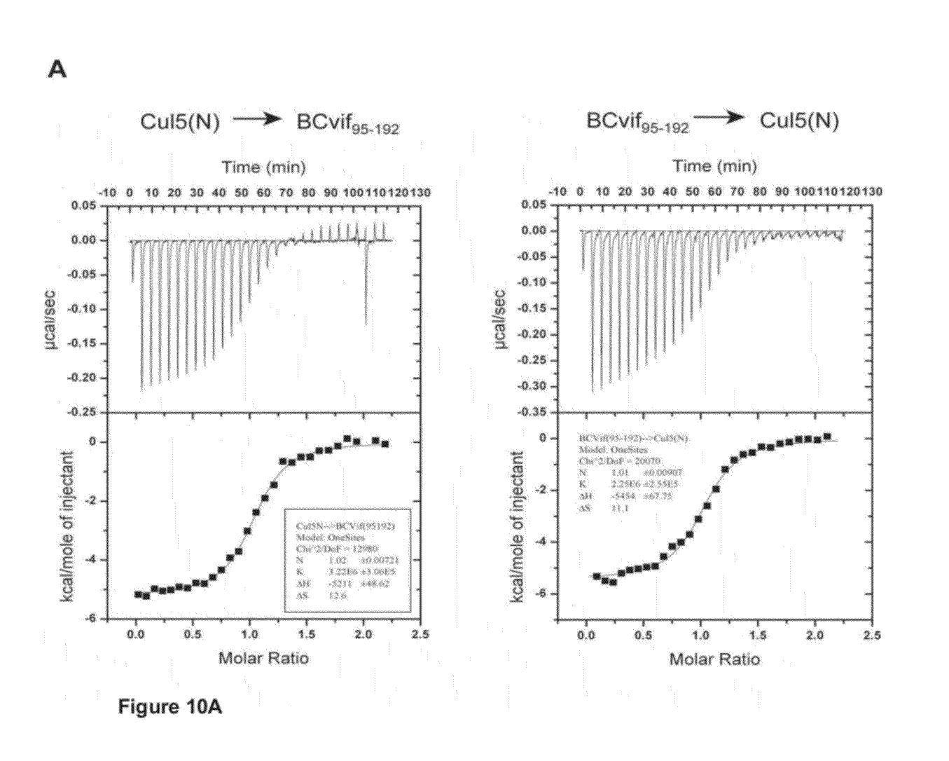

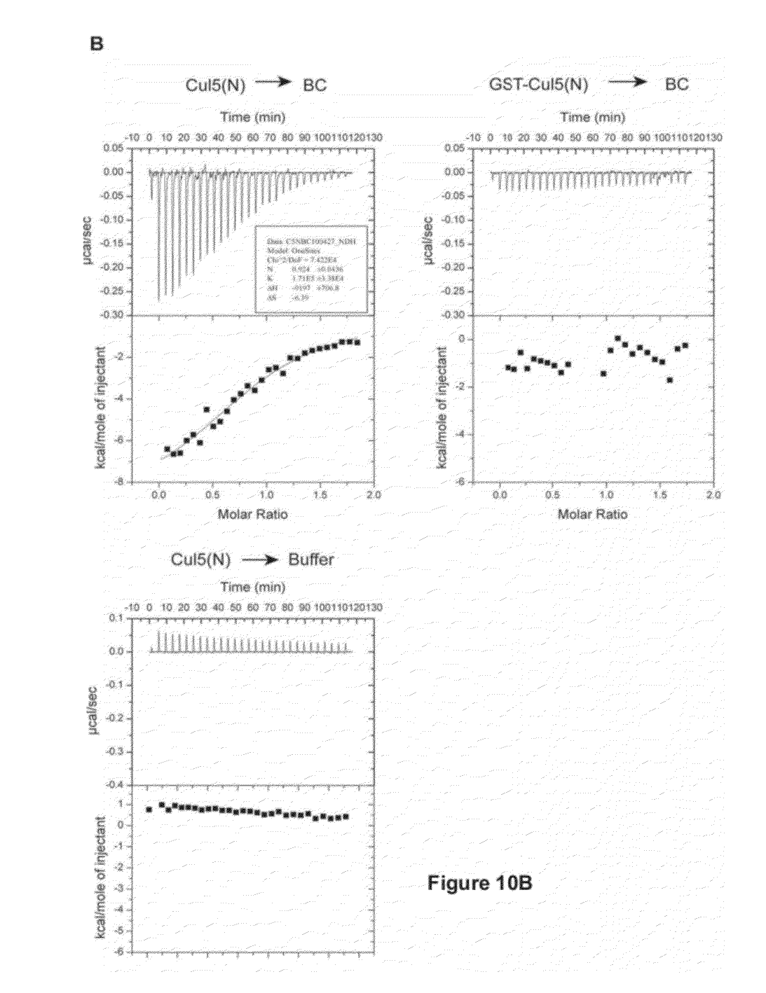

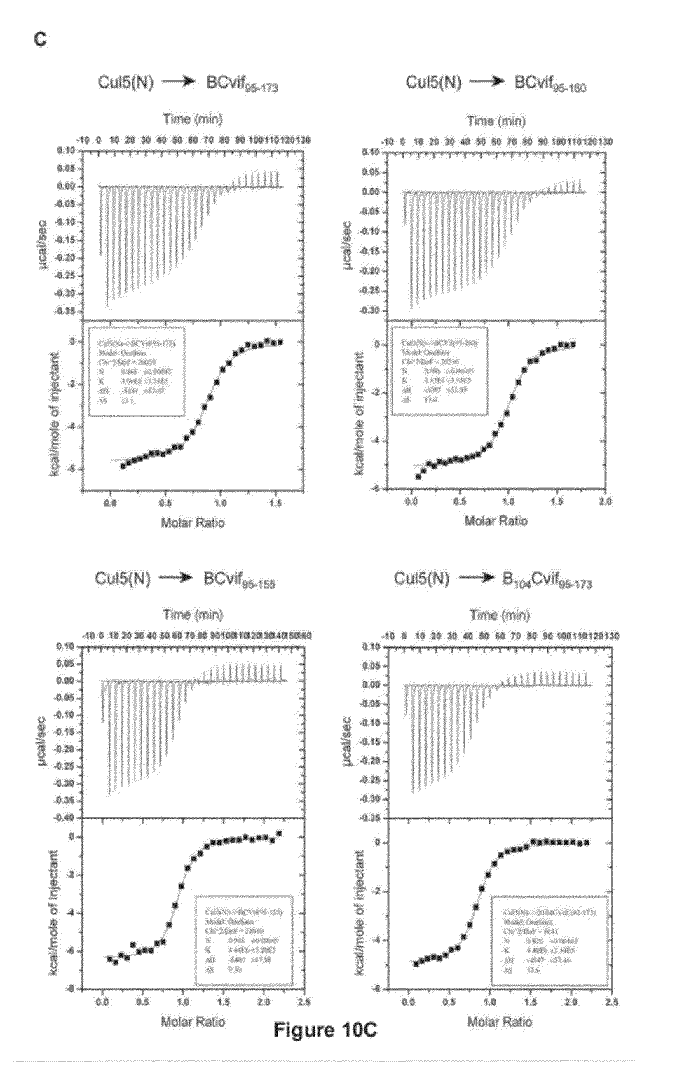

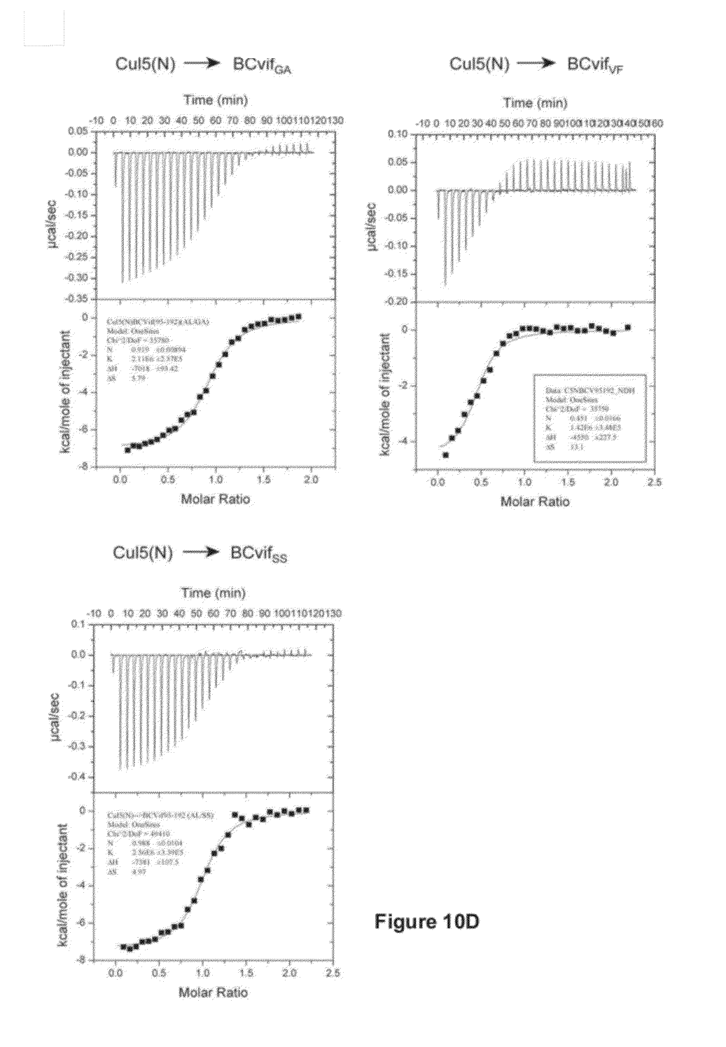

Interestingly, the heat of dilution of Cul5(N) into buffer was endothermic (average heat of injection=638±185 cal mol⁻¹). Thus, it was not surprising that upon reaching saturation, the heats of injection for titrations of Cul5(N) into BC or EloBCvif complexes were also endothermic. All data were fit with the one set of sites binding model to determine the parameters K_{A} (1/K_{D}), n, and ΔH using the Origin 7.0 software package (MicroCal). AG was calculated from ΔG=−RTlnK_{A}, where R=1.98722 cal K⁻¹ mol⁻¹, T=absolute temperature (Kelvin). ΔS was calculated from the equation, ΔG=ΔH−TΔS. The reported thermodynamic parameters and associated errors are the average and standard deviation from at least two replicates, except for the titration of BCvif₉₅₋₁₉₂ into Cul5(N) and the titration of GST-Cul5(N) into BC, which were done once. Errors for the thermodynamic parameters and the calculated χ²/degrees-of-freedom are shown as insets within the binding isotherms for representative individual experiments (FIG. 10).

### Sedimentation Velocity Analytical Ultracentrifugation of EloB-EloC-Vif Ternary Complexes.

Sedimentation velocity analytical ultracentrifugation (SV-AUC) experiments were performed with a Beckman Coulter ProteomeLab XL-A analytical ultracentrifuge equipped with absorbance optics and an eight-hole An-50 Ti analytical rotor. Sedimentation velocity experiments were carried out at 10° C. and 50,000 rpm (200,000×g) using 3-mm two-sector charcoal-filled Epon centerpieces with quartz windows. Three different concentrations ˜0.2 to 1.8 mg ml⁻¹ of each complex were diluted from respective stock solutions and analyzed. Each sample was scanned for absorbance at 230 nm for 300 total scans. Sedimentation boundaries were analyzed by the continuous sedimentation coefficient distribution (c(s)) method using SEDFIT.

Probing the EloBC-vif Complex Interaction with the N-Terminal Domain of Cul5

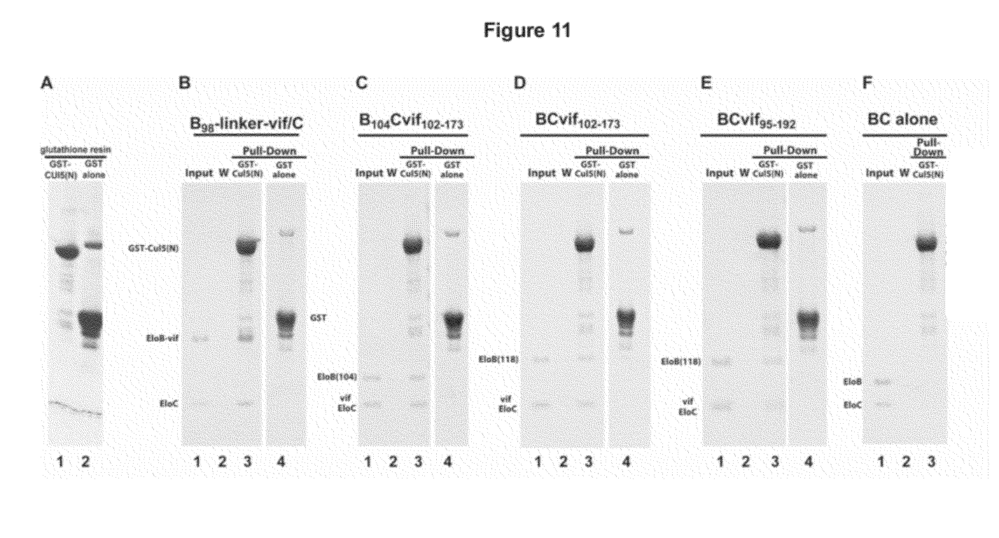

The N-terminal domain of human Cul5 was established to be necessary and sufficient for binding to cellular EloBC-SOCS-box protein complexes (Babon et al., 2009 J Mol Biol 387, 162-174). To establish comparable binding and boundary requirements of the HIV-1 protein vif, GST-Cul5(N) pull-down assays using purified EloBC-vif complexes in which variable lengths where chosen for the respective EloB and vif proteins were used. The specific ternary complexes tested included: EloB₁₋₁₀₄-EloC₁₇₋₁₁₂-vif₁₀₂₋₁₇₃, EloB₁₋₁₁₈-EloC₁₇₋₁₁₂-vif₁₀₂₋₁₇₃, and EloB₁₋₁₁₈-EloC₁₇₋₁₁₂-vif₉₅₋₁₇₃. Each complex demonstrated binding to GST-Cul5(N) but not GST alone (FIG. 11C-11E). Furthermore, the EloBC heterodimer did not bind GST-Cul5(N) (FIG. 11F). The binding results suggested that each vif truncation was sufficient to bind Cul5. Likewise, the EloB₁₋₉₈(linker)vif₁₀₂₋₁₇₃-EloC complex also bound Cul5(N), and indicated that the vif moiety is positioned in a biologically active conformation despite covalent attachment to the C-terminal of EloB (FIG. 11B). This observation lends credibility to the approach of expressing the EloB-linker-vif as a fusion protein, which was necessary to surmount solubility problems. These results also suggest that the C-terminal segment of EloB from 105 to 118 is expendable for formation of the EloBCvif complex. The interaction between Cul5(N) with isolated vif was not tested because it was not possible to purify appreciable amounts of the vif protein in the absence of EloBC. The observation that key complexes of EloBCvif were capable of cognate interactions prompted us to quantify the equilibrium binding and thermodynamic parameters of the Cul5(N) interaction with the tripartite complex using ITC.

Quantifying the EloBC-vif Interaction with Cul5(N)

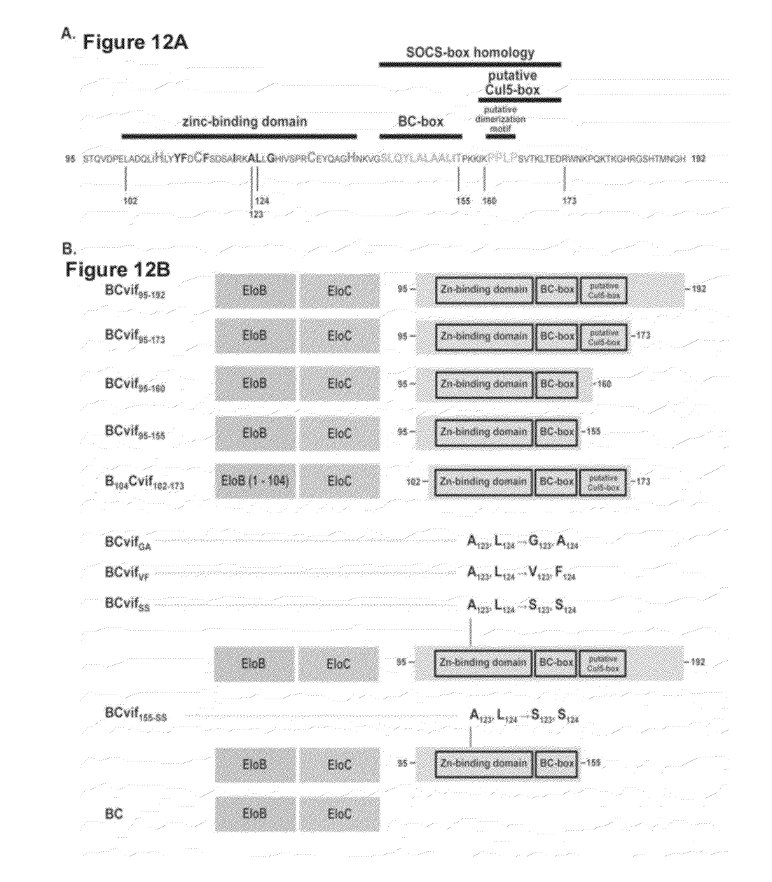

ITC was used to assess the affinity and thermodynamic characteristics of binding between human Cul5(N) and preformed EloBCvif complexes (FIG. 10A-10D). To probe regions of polypeptide chains that are important in formation of the quaternary complex, the length of the vif protein, as well as that of EloB, were truncated at the boundaries reported for various functional motifs (FIG. 12). Here the complex comprising full-length EloB, EloC₁₇₋₁₁₂, and the entire C-terminal domain of vif (vif₉₅₋₁₉₂)—herein called BCvif₉₅₋₁₉₂ was first tested. This complex demonstrated reasonable affinity for Cul5(N), displaying a K_{d} of 327±40 nM (FIG. 10A). At 30° C. the interaction was thermodynamically favorable both enthalpically (ΔH=−5.24±0.41 kcal mol⁻¹) and entropically (ΔS=12.42±1.51 kcal mol⁻¹ K⁻¹). Importantly, ITC titrations with an opposite injection order where BCvif₉₅₋₁₉₂ was titrated into Cul5(N) produced comparable affinity (K_{d} of 420 nM) thermodynamic parameters and binding stoichiometry. The 1:1 binding stoichiometry observed here for the EloBCvif interaction with Cul5 is consistent with the binding stoichiometry of complexes comprising EloBC with cellular SOCS-box proteins and Cul5 (Babon et al., 2009 J Mol Biol 387, 162-174).

### EloBC Binding to Human Cul5(N) in the Absence of a GST Tag

Titration of Cul5(N) into EloBC (without vif) (FIG. 10B) revealed a lower affinity (K_{d}=6349 nM) than when the C-terminal half of vif was present (K_{d}=327±40 nM), affirming the importance of vif in promoting the interaction of EloBC with Cul5. Prior work from the laboratory of Raymod Norton (Babon et al., 2009 J Mol Biol 387, 162-174), as well as results presented herein (FIG. 11F), demonstrated that GST-Cul5(N) was incapable of supporting an interaction with the EloBC heterodimer in the absence of a SOCS-box protein. By contrast, our ITC experiments, using untagged Cul5(N) demonstrated a measurable, albeit low affinity, interaction with EloBC does occur without a bound SOCS-box protein. Conversely, when GST-Cul5(N) was titrated into EloBC, no interaction was observed (FIG. 10B). This result suggests that the presence of GST and/or the linker regions of our constructs can sterically hinder access to BC when covalently bound to the N-terminus of Cul5. These observations may account for why the GST-Cul5 pull-down experiments by Babon et al. (2009 J Mol Biol 387, 162-174) indicated BC cannot bind Cul5 in the absence of a SOCS-box protein.

Vif Truncations to Probe the Cul5-Box Interaction with Cul5(N)

The binding affinity of Cul5(N) to EloBCvif complexes was assessed by truncating the vif C-terminal polypeptide (FIG. 10C). These constructs included vif₉₅₋₁₇₃, vif₉₅₋₁₆₀, and vif₉₅₋₁₅₅. The binding affinity between Cul5(N) and BCvif₉₅₋₁₇₃ exhibited a K_{d} of 337±15 nM, which is not significantly different from that with BCvif₉₅₋₁₉₂. This observation suggests that the last 20 residues of vif do not contribute significantly to binding between BCvif₉₅₋₁₉₂ and Cul5(N). In contrast, slightly higher affinity was observed between Cul5(N) and BCvif₉₅₋₁₆₀ (K_{d}=285±14 nM) relative to BCvif₉₅₋₁₉₂. This apparent enhancement of affinity was interesting since the excluded region of vif from 160 to 173 encompasses most of the putative Cul5-box, which is a key binding determinant of cell-based SOCS-boxes proteins. As such, the entire Cul5-box up to the BC-box of the vif C-terminus was removed, which produced stronger affinity between Cul5(N) and BCvif₉₅₋₁₅₅ (K_{d}=213±17 nM). Importantly, our results demonstrate that the Cul5-box (vif residues 159-173)—which encompasses the ¹⁶¹PPLP¹⁶⁴ motif—is not functionally operative in Cul5 binding. Although the interaction between Cul5(N) and BCvif₉₅₋₁₆₀ (K_{d}=285 nM) was weaker than the interaction between Cul5(N) and BCvif₉₅₋₁₅₅ (K_{d}=213 nM), it was still somewhat stronger than the interaction between Cul5(N) and EloBCvif complexes with longer vif termini (K_{d}=337±15 nM for BCvif₉₅₋₁₇₃ and K_{d}=327±40 nM for BCvif₉₅₋₁₉₂). Perhaps most significantly, this work attributes binding between vif and Cul5(N) to regions primarily within the zinc-binding HCCH domain of vif. While the BC-box of vif is present in all vif constructs, the structural model (PDB ID 3DCG) shows it is buried in an interface with EloC and thus not available for mediating an interaction with Cul5(N).

### The Role of Conserved Residues in the HCCH Motif for Cul5(N) Binding

Since the HCCH region of vif appears sufficient for Cul5(N) binding, experiments were focused on the conserved residues within the HCCH zinc-binding domain: H₁₀₈-x₂-Y₁₁₁F₁₁₂-x-C₁₁₄F₁₁₅-x₄-(I/V)₁₂₀-x₂-A₁₂₃(L/I/V)₁₂₄-x-G₁₂₆-x₆-C₁₃₃-x₅-H₁₃₉. The conserved hydrophobic residues Y₁₁₁F₁₁₂, F₁₁₅, I₁₂₀, and A₁₂₃L₁₂₄ have been implicated in promoting vif interaction with Cul5(N), but their precise roles have not been determined (Xiao et al., 2006 Virology 349, 290-299). The results of co-immunoprecipitation assays demonstrated that the respective vif mutants Y111A/F112A, F115A, I120S, or A123S/L124S completely abrogated Cul5 binding and APOBEC3G degradation, and HIV-1 infectivity. To quantify the contribution of a particular set of conserved residues to Cul5 binding, mutations were incorporated into the conserved tandem residue pair, A123/L124, in the context of a BCvif₉₅₋₁₉₂ complex (FIG. 10D) and examined the effect on Cul5(N) binding using ITC. Site-directed mutants were generated in vif₉₅₋₁₉₂ in the following combinations (vif mutant tripartite complex nomenclature shown in parenthesis): A123V/L124F (BCvif_{VF}), A123G/L124A (BCvif_{GA}), and A123S/L124S (BCvif_{SS}). These mutations were chosen to assess the affect of adding (VF) and removing (GA) bulk from the tandem pair, while the A123S/L123S double mutant was chosen to assess the affect of switching the residue pair from hydrophobic to hydrophilic. The mutant complex, with added bulk, BCvif_{VF} exhibited a K_{d} of 847 nM±202 nM for Cul5(N). This level of binding is reduced 2.5 fold from that of the wild-type complex (K_{d} of 327 nM±40 nM). The less bulky mutant complex BCvif_{GA} produced a K_{d} of 562 nM±95 nM, which is evidence for lower Cul(N) affinity but not as substantial a loss of affinity as the bulkier substitutions in the BCvif_{VF} mutant promoted. The tandem polar serine residue mutant (BCvif_{SS}) had the least impact, resulting in a K_{d} of 397 nM±9 nM, which may have implications for the structural organization of the vif C-terminal region.

Although BCvif_{SS} bound to Cul5 with nearly the same affinity as wildtype, the respective thermodynamic parameters differed. The value of ΔS upon binding Cul5(N) is 12.42 kcal mol⁻¹ K⁻¹ for the BCvif wild-type complex and 5.28 kcal mol⁻¹ K⁻¹ for the BCvif_{SS} complex. This change in ΔS may reflect the entropic difference between unbound wild type vif harboring solvent-exposed hydrophobic A123/L124 residues versus hydrophilic S123/S124 residues. The ΔH values associated with wild type BCvif and BCvif_{SS} binding to Cul5 are −5.24 kcal mol-1 and −7.28 kcal mol⁻¹, respectively. The increased magnitude of ΔH for binding of the BCvif_{SS} mutant to Cul5(N) over that of WT (AL) is consistent with an increase in the number of hydrogen bonds between the hydroxyl moieties of the Ser side-chains and Cul5 residues (O'Brien, R., Ladbury, J. E., and Chowdhry, B. Z. (2001) Isothermal titration calorimetry of biomolecules, in Protein-Ligand Interactions: hydrodynamics and calorimetry (Harding, S. E., and Chowdhry, B. Z., Eds.), pp 263-286, Oxford University Press, Oxford). The ability of both the wild type vif (AL) and mutant vif (SS) to support binding with Cul5 suggests that the binding interface may be flexible and capable of adopting slightly different arrangements. Alternatively, these data could suggest that the conserved A123/L124 residue pair of vif is not the primary determinants of Cul5 binding.

Using the one set of sites binding model, the binding stoichiometries for the Cul5:BCvif_{GA} and Cul5:BCvif_{SS} interactions were ˜1:1 with calculated n values of 0.90 and 1.01 respectively, whereas, the binding stoichiometry for Cul5:BCvif_{VF} was ˜2:1 (n=0.43).

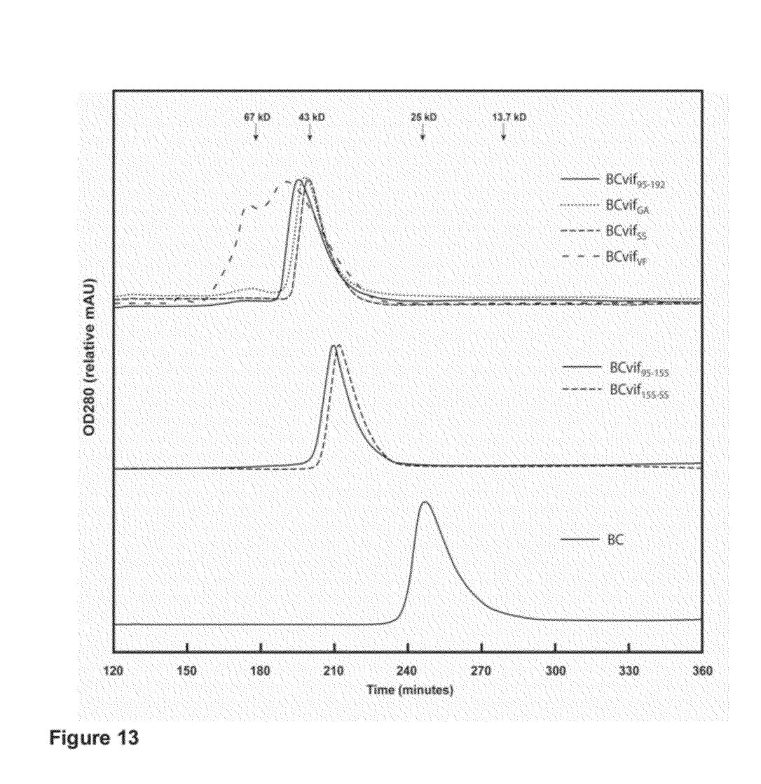

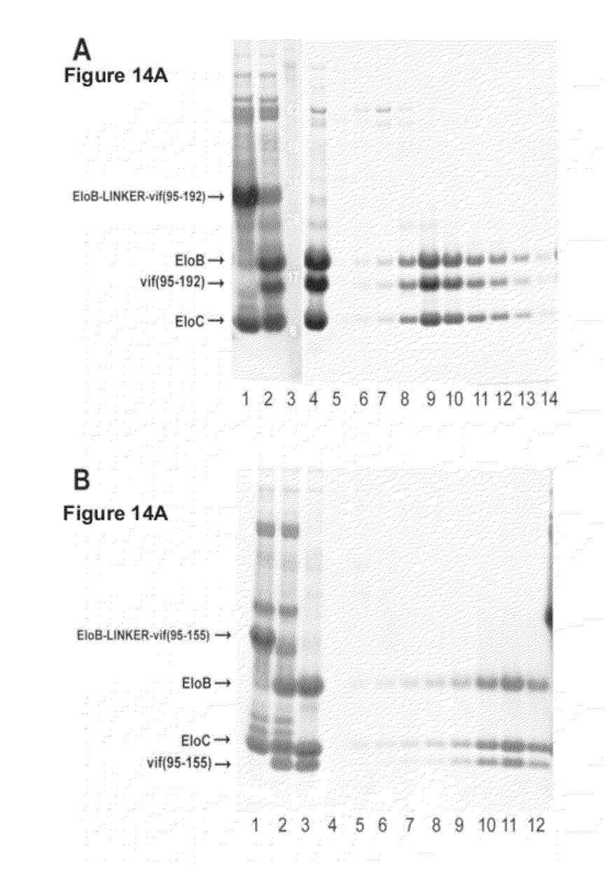

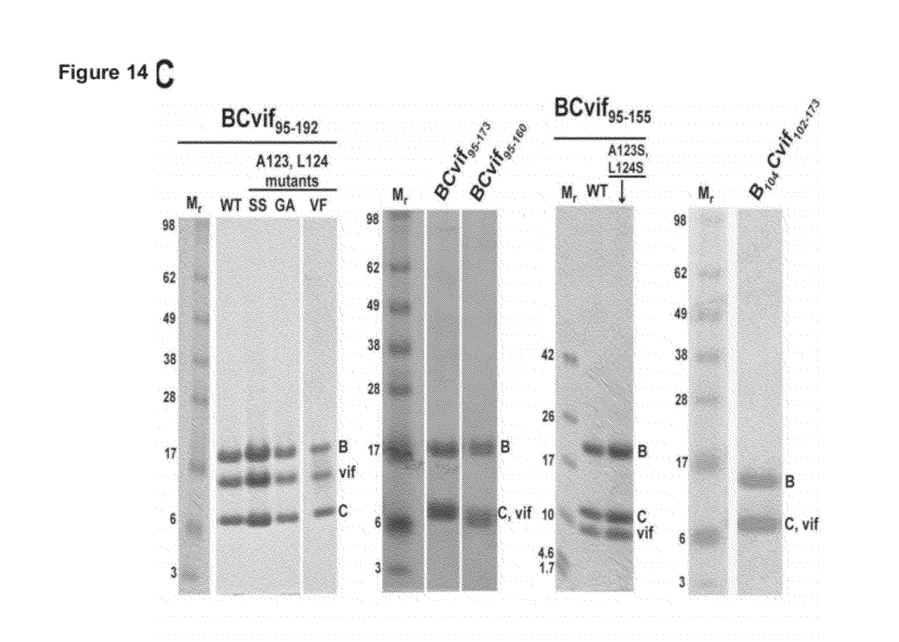

This 2:1 stoichiometry for the Cul5:BCvif_{VF} interaction was unexpected and prompted fitting the data with other binding site models; however, all other models also did not satisfactorily fit the data. Interestingly, the size exclusion chromatography profile of the BCvif_{VF} complex revealed conformational heterogeneity that was not present for wild-type or other mutant vif complexes (FIG. 13). DLS analysis of the mutated BCvif_{VF} complex also revealed that it self-associates to form higher order oligomers in the molecular weight range of 2 to 3×10² kD. These changes are likely due to the increase in hydrophobic bulk of BCvif_{VF} compared to wild-type complex. Thus, one possible explanation for the lower n value is that the aggregation of protein may have rendered some of the BCvif_{VF} complex unavailable for interaction with Cul5(N), thereby lowering the effective protein concentration in solution and artificially lowering the calculated n value. To test this, the data was applied to a one set of sites binding model with the concentration of BCvif_{VF} set to half the actual concentration. As expected, the calculated value of n doubled from 0.451 to 0.901—suggesting a 1:1 binding stoichiometry, while the calculated thermodynamic parameters remained unchanged. However, without a quantitative analysis of the actual distribution of conformations of the BCvif_{VF} complex, it is not possible to accurately fit the binding isotherm for the interaction between BCvif_{VF} complex and Cul5(N). Furthermore, a second possible explanation for the lower n value is that the A123V/L124F mutation caused vif to misfold; however, this possibility seems less likely because (i) no dissociation of the tripartite complex is observed (by SEC), indicating that the core fold of vif is intact and capable of promoting interaction with BC, and (ii) the tripartite mutant complex, BCvif_{VF}, is still able to bind Cul5 with greater affinity than BC alone, suggesting vif elements that mediate the interaction with Cul5 are still poised in the proper orientation.

Role of EloB C-Terminal Residues in the Interaction of EloBCvif with Cul5(N)

At present, the reported crystal structures of EloBC-SOCS-box ternary complexes indicate that the C-terminal-most 14 residues of EloB (105-118) are disordered as indicated by the absence of electron density (Bullock et al. 2006 Proc Natl Acad Sci USA 103, 7637-7642; Babon et al., 2008 J Mol Biol 381, 928-940; Bullock et al., 2007 Structure 15, 1493-1504). This result prompted the exploration of the necessity of the EloB C-terminus in the context of EloBC binding activity. Accordingly, an EloBCvif complex comprising EloB₁₋₁₀₄, EloC₁₇₋₁₁₂, and vif₁₀₂₋₁₇₃ was purified and measured the affinity for Cul5 using ITC. The resulting K_{d} of 288±14 nM was on par with that measured for complexes comprising full length EloB, suggesting that at least the last 14 residues of EloB are dispensable for formation of a complex involving a viral protein (i.e. EloBCvif) and binding of Cul5.

### Size-Exclusion Chromatographic Analysis of EloBCvif Complexes

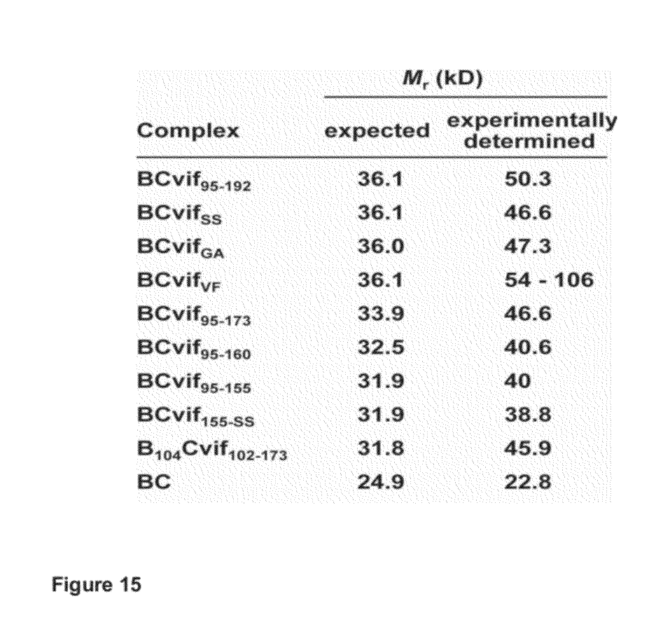

The EloBCvif complexes of this investigation are illustrated in FIG. 12. After S-100 size-exclusion chromatography, the EloBCvif complexes are estimated (by coomassie-stained SDS-PAGE) to be greater than 95% pure (FIG. 14C). Each of the ternary complexes was subjected to size exclusion chromatography producing a single peak (FIG. 13). One exception was the BCV_{VF}, which exhibited a broad elution profile consistent with several high molecular mass species. The aberrantly high mass was likely due to self-association that arose from increased hydrophobicity of the mutant Val and Phe residues relative to wildtype Ala and Leu. No evidence for dissociation of any of the complexes into substituent components was observed, suggesting that vif, as purified here, associates tightly with the EloBC heterodimer. The molecular mass of each EloBCvif complex calculated from the elution profile was slightly greater than expected for 1:1:1 stoichiometry (FIG. 15). By contrast, the BC complex eluted at the expected molecular weight of the 1:1 heterodimer. These observations prompted us to interrogate the oligomeric state of EloBCvif using analytical ultracentrifugation (AUC).

### Vif Oligomerization is Mediated by the Zinc-Binding Motif

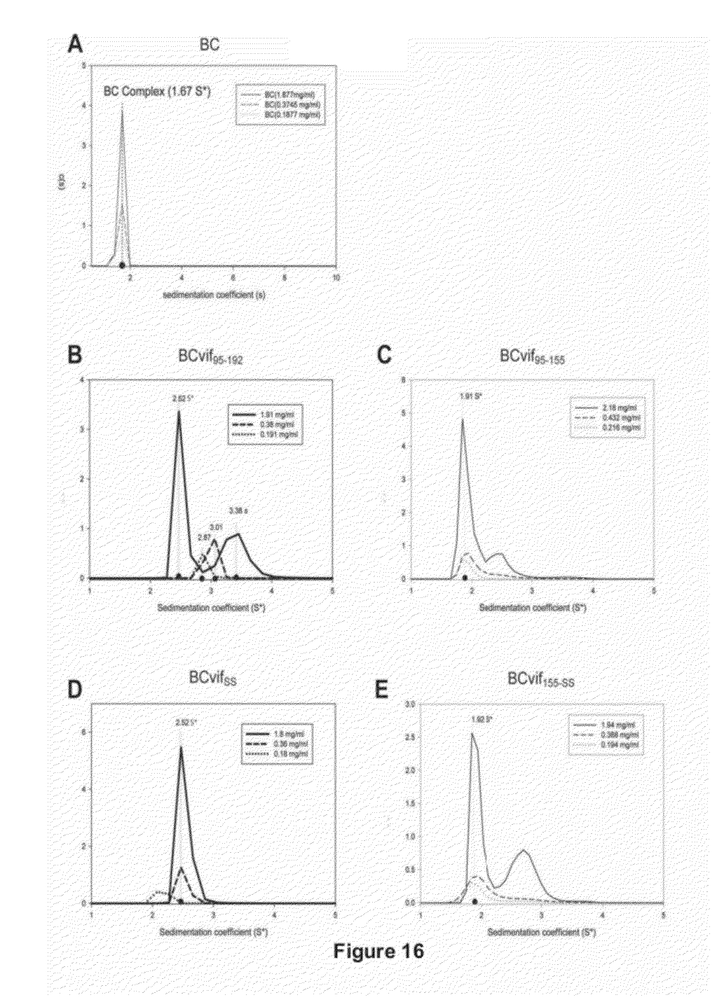

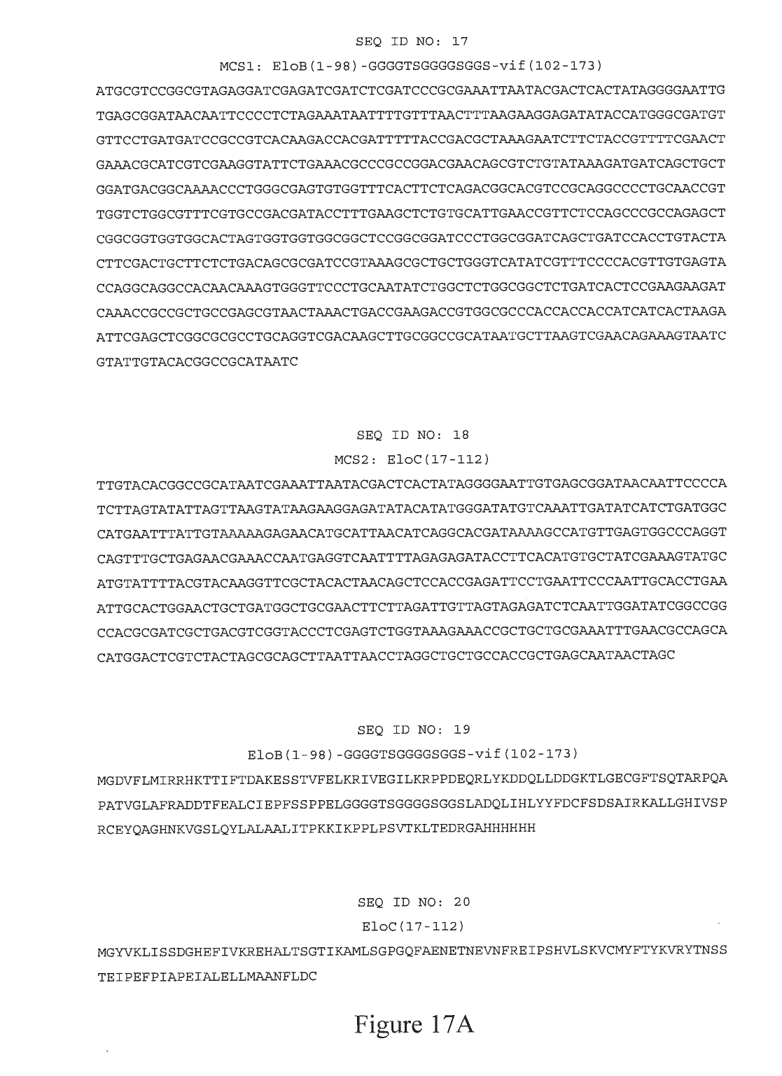

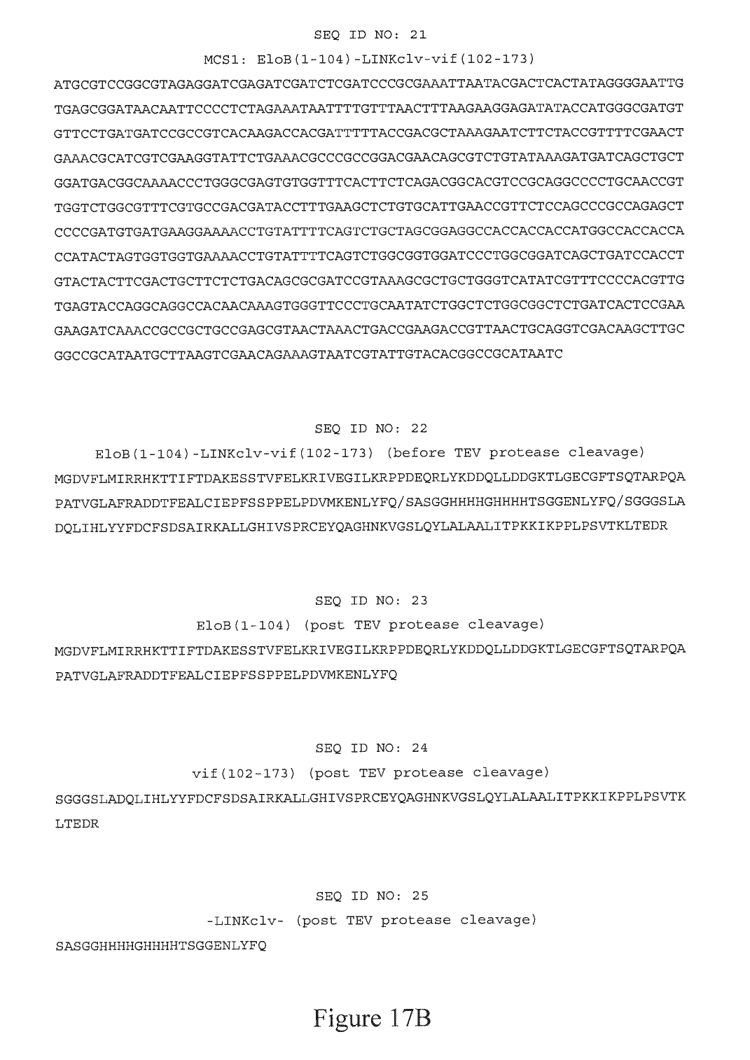

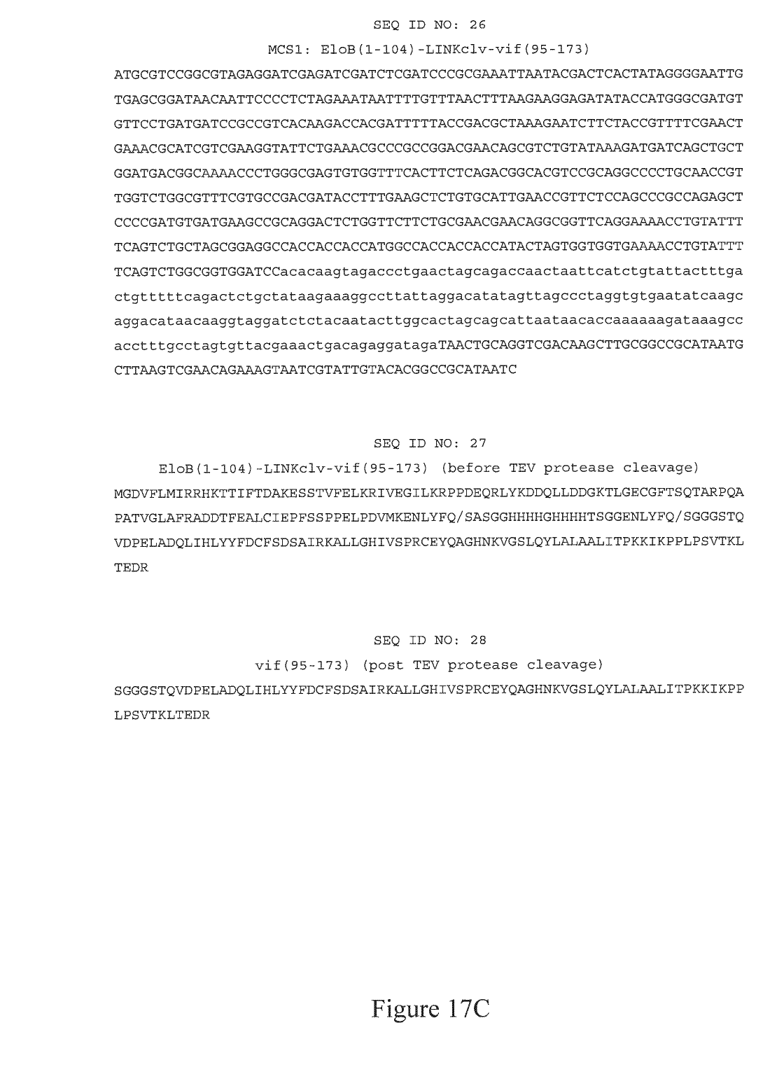

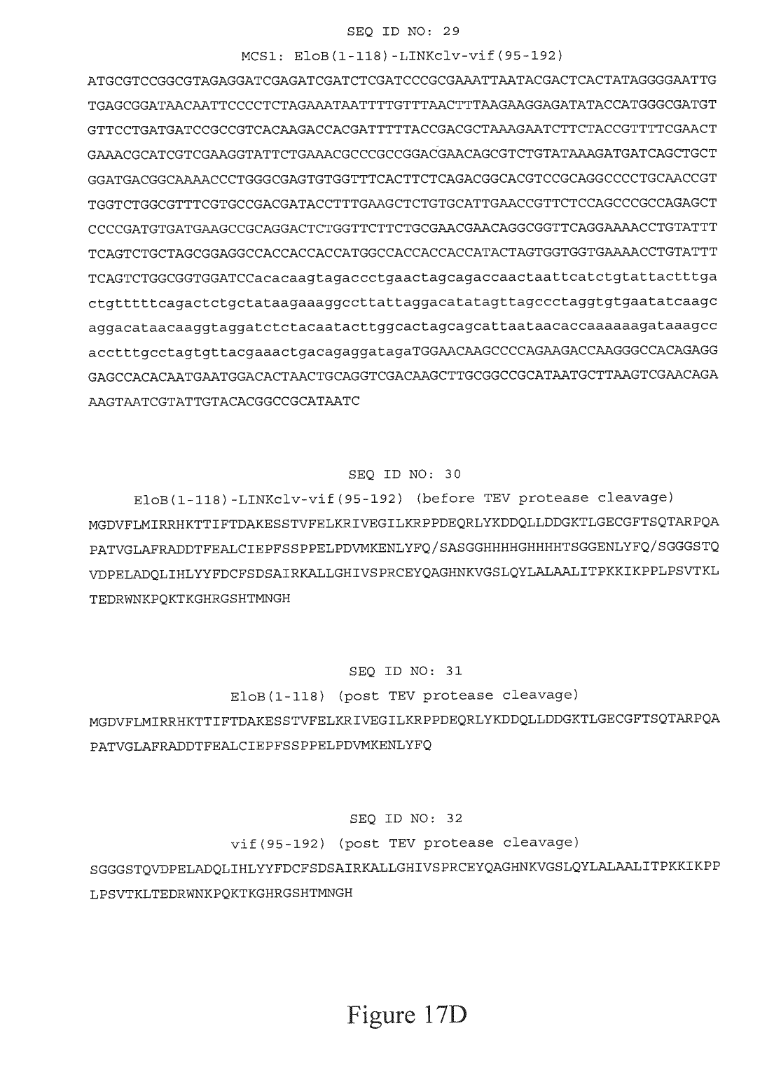

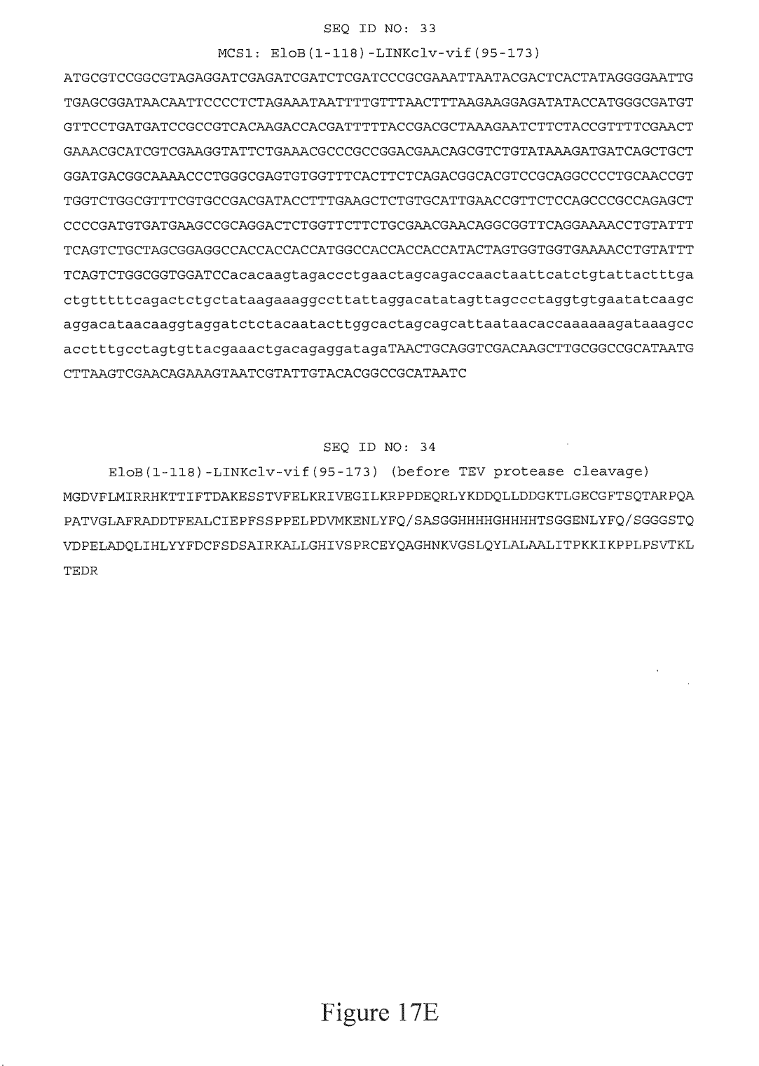

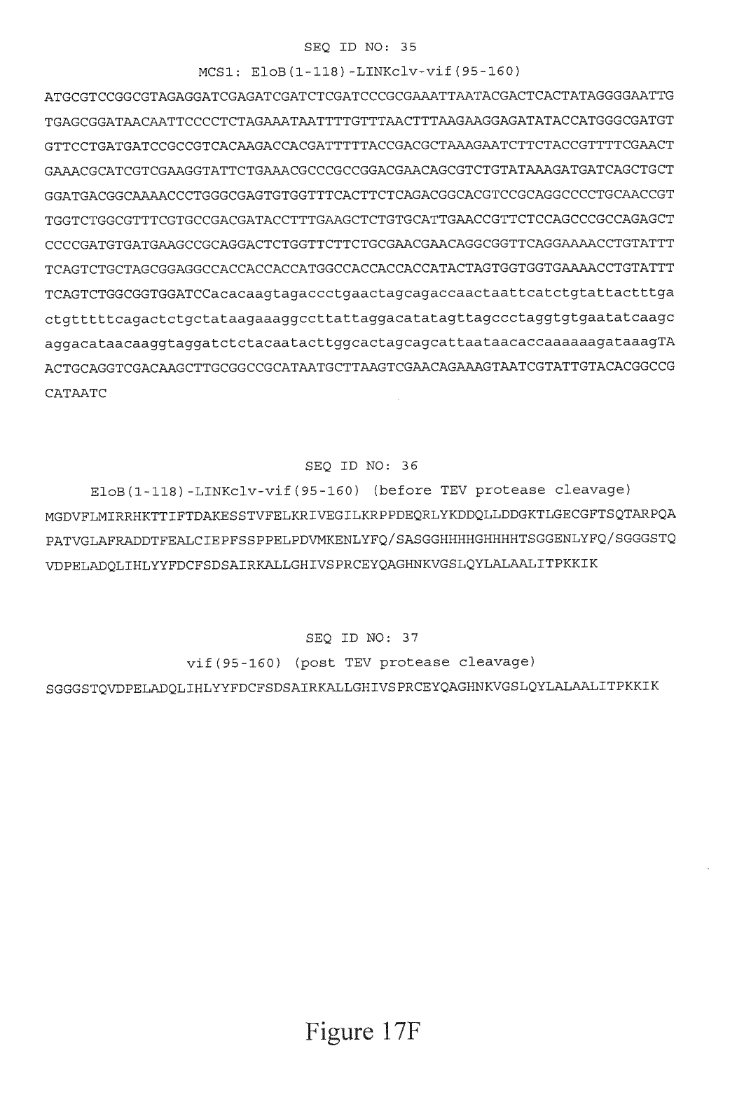

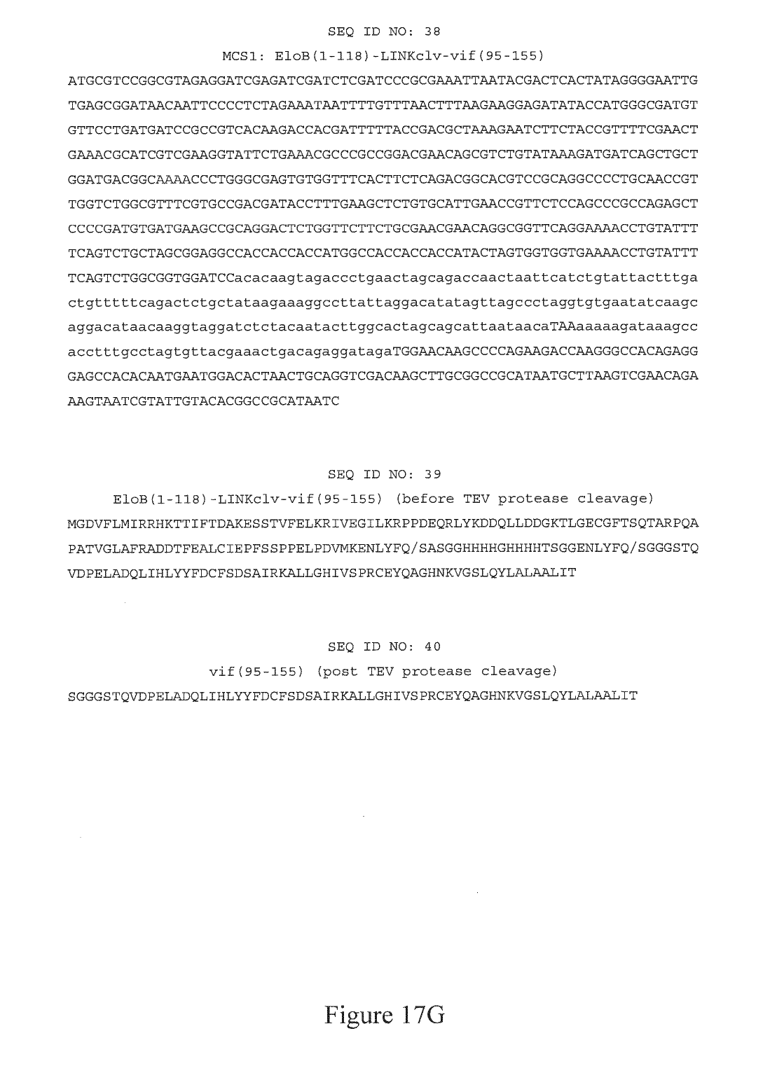

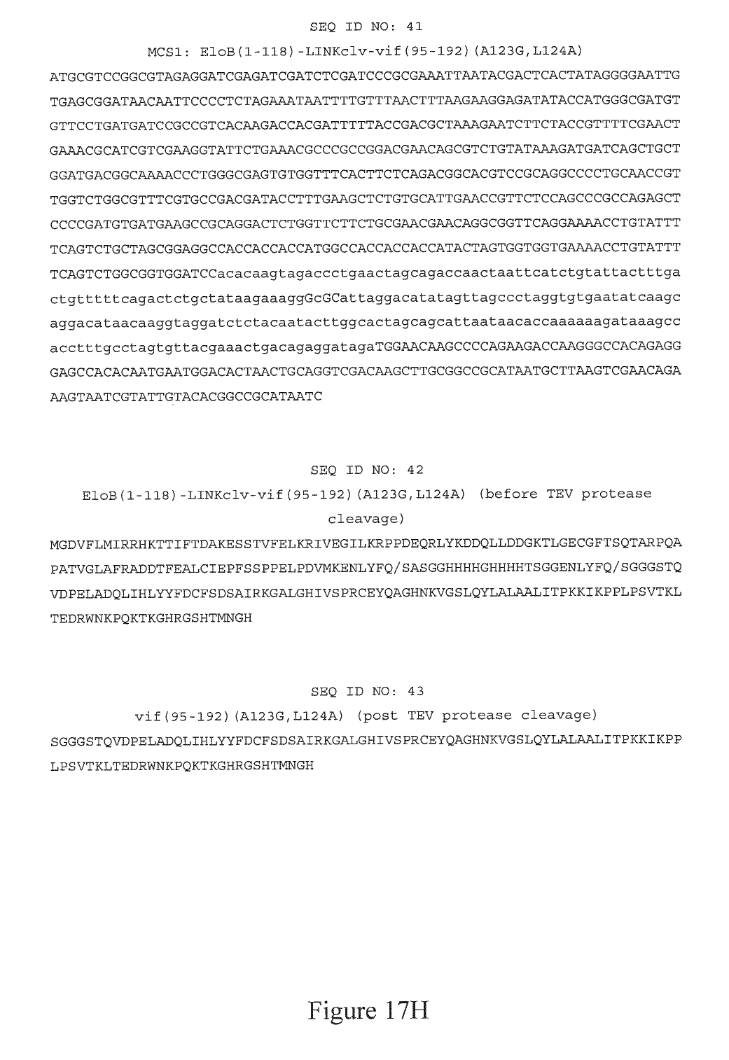

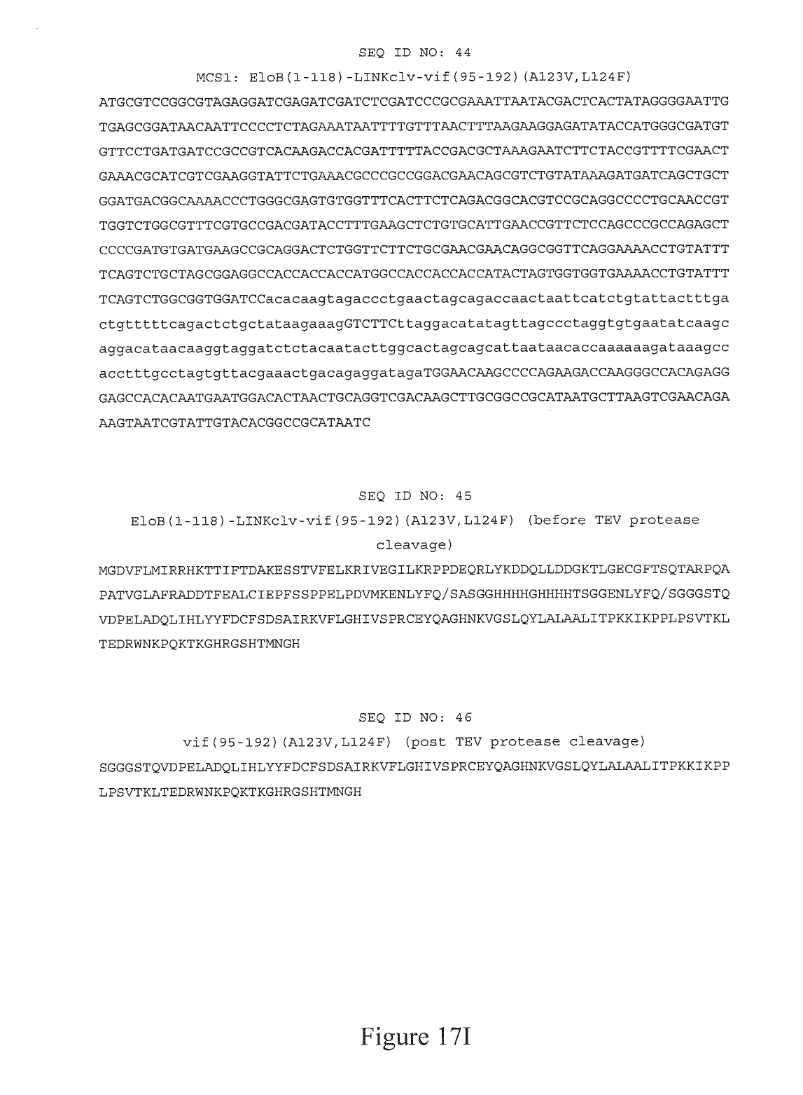

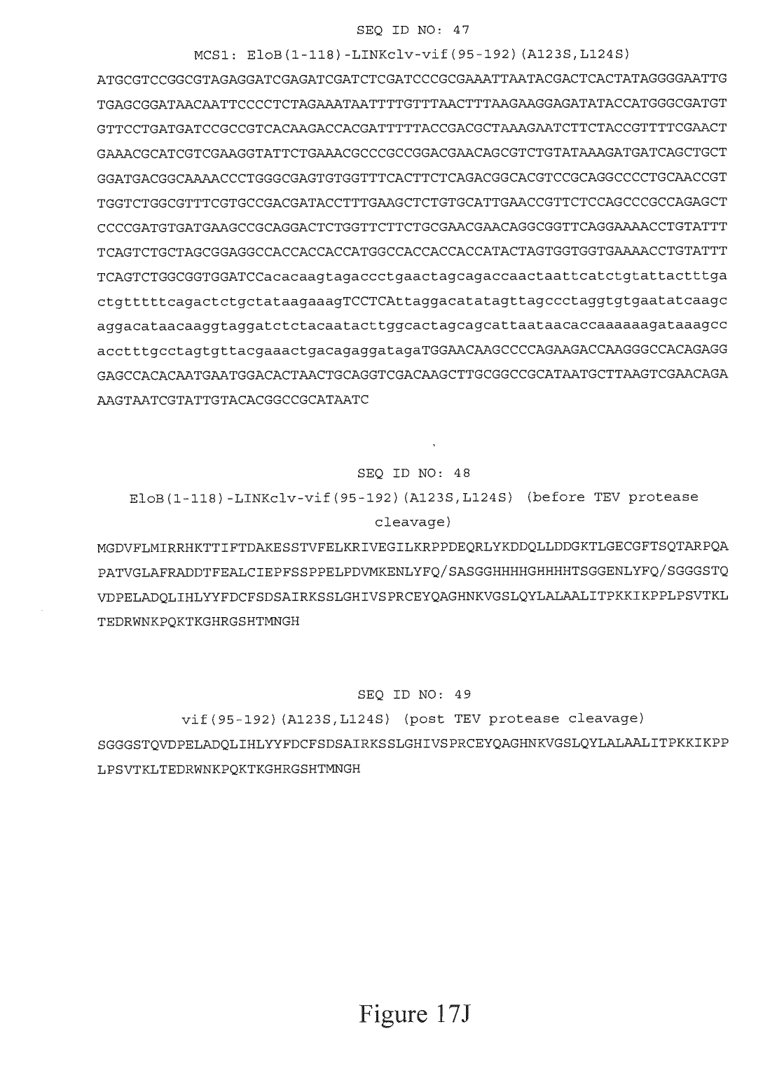

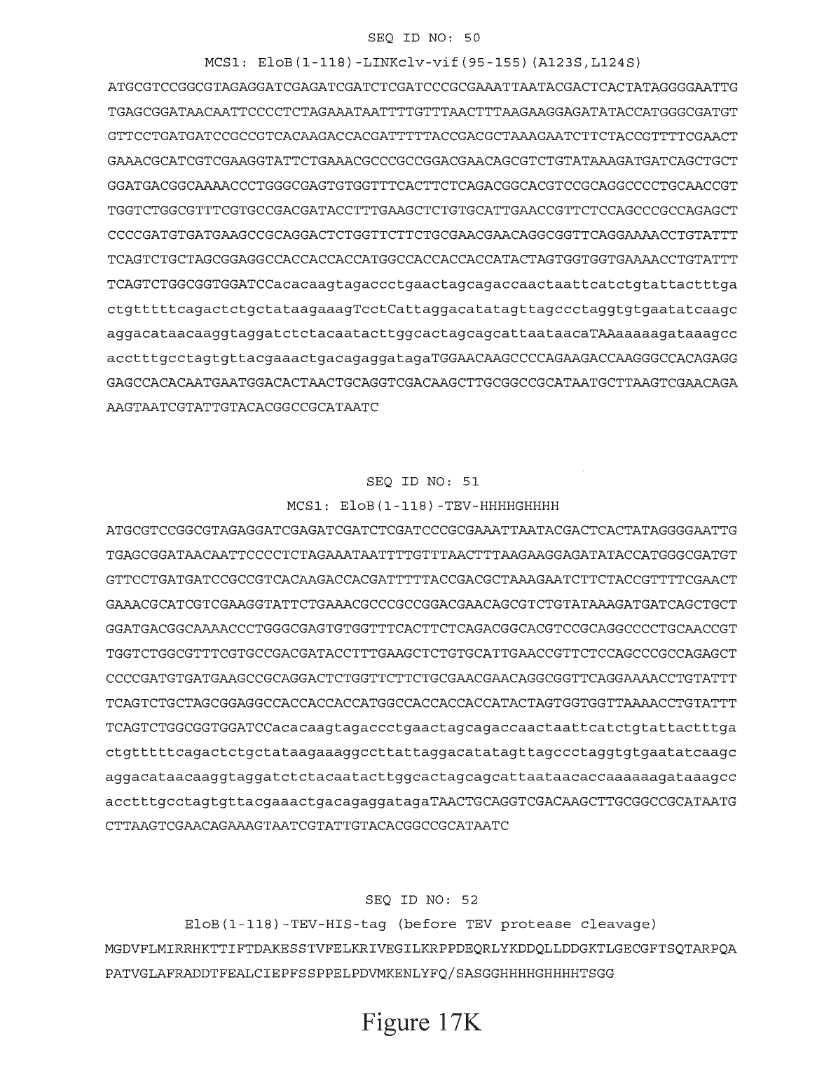

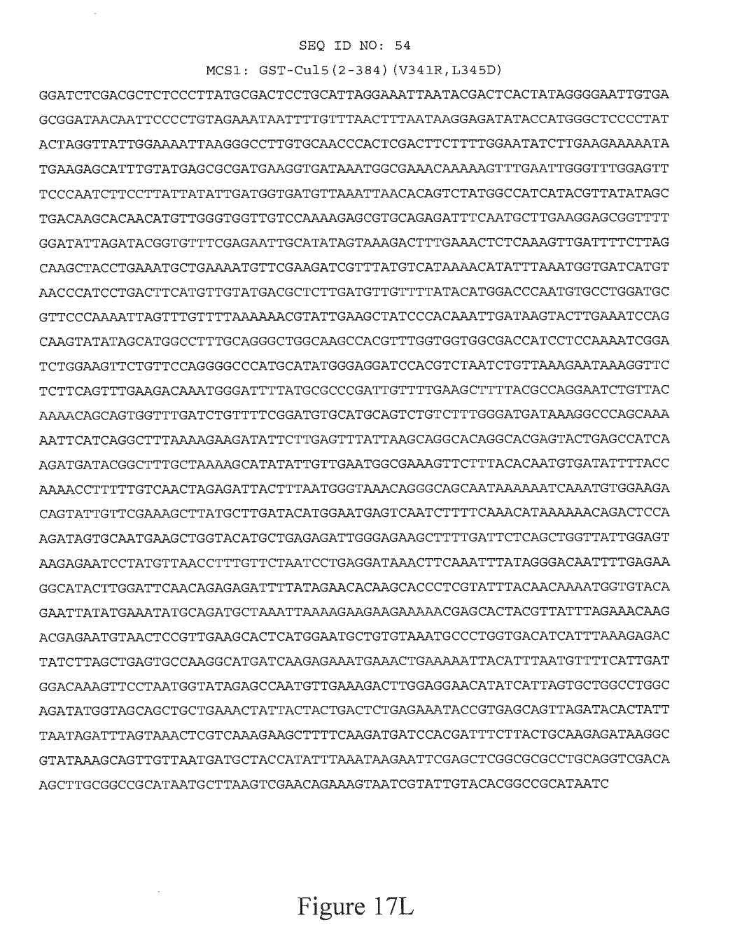

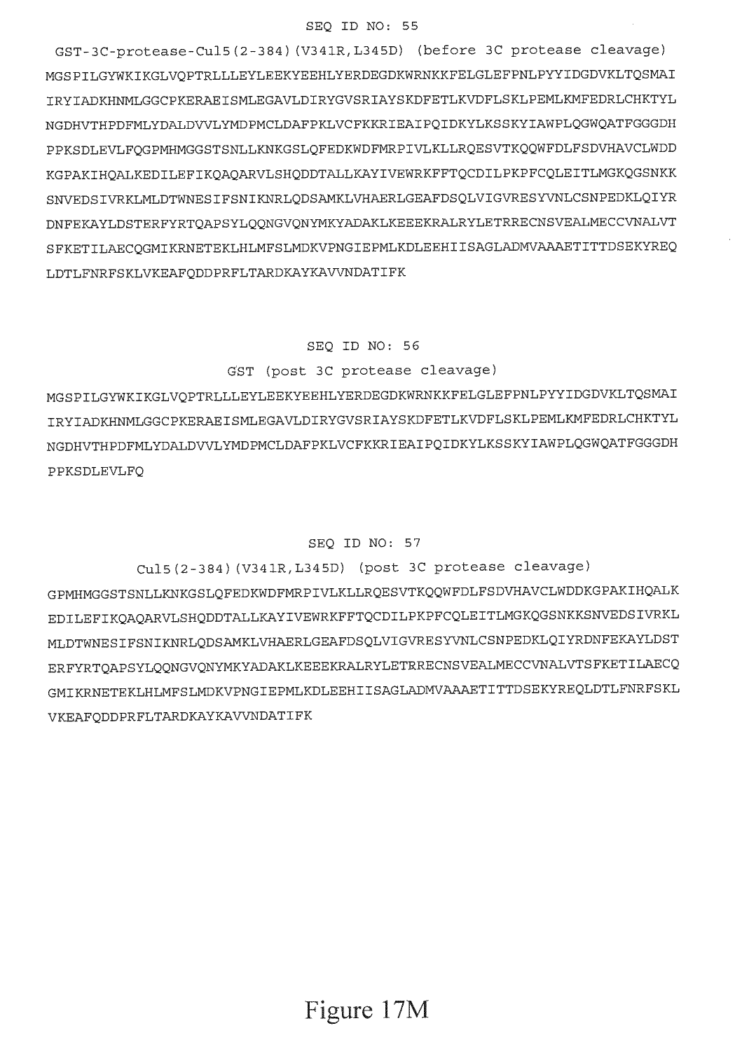

Previous work has demonstrated that vif is a self-interacting protein (Yang et al., 2001 J Biol Chem 276, 4889-4893) and that oligomerization is necessary for vif biological activity (Miller et al., 2007 Retrovirology 4, 81). Phage display analysis identified the ¹⁶¹PPLP¹⁶⁴ sequence, within the putative Cul5-box as a central determinant in vif oligomerization. The vif Cul5-box has been demonstrated herein to be dispensable for human Cul5(N) binding. Absence of clear evidence regarding the oligomeric state of EloBCvif complexes from SEC analyses (FIGS. 14 and 16) compelled us to perform a concentration-dependent analysis of our complexes using sedimentation velocity AUC techniques. The purified heterodimer, BC was first examined (FIG. 16A). As expected, the BC complex was monodisperse and the experimental sedimentation coefficients identical (1.67 s*) from 3 concentrations over a 10-fold range (0.19, 0.37, and 1.88 mg ml⁻¹). To assess vif-mediated oligomerization, the BCvif₉₅₋₁₉₂ complex was examined. The results revealed that BCvif₉₅₋₁₉₂ behaves as a self-associating molecule in which complexes coexist as a mixture of dimers and isolated tripartite complexes (FIG. 16B). The single sedimentation coefficient peaks, 2.87 and 3.01 S*, for the two lowest concentrations (0.19 and 0.38 mg ml⁻¹ respectively) are indicative of a fast interconversion rate between dimers and isolated complexes compared to the timescale of sedimentation. At the highest concentration analyzed (1.91 mg ml⁻¹), distinguished sedimentation coefficient peaks for the dimer (3.38 S*) and isolated complex (2.52 S*) are observed. This is likely due to the enhanced signal-to-noise ratio or to cooperative binding, which could stabilize the oligomers on the timescale of sedimentation. Importantly, no dissolution of the fundamental tripartite complex into isolated EloB, EloC and vif subunits was observed over the concentration range of the experiments. The molecular masses predicted from individual sedimentation coefficient peaks at the highest concentration analyzed agree well with the molecular masses of dimeric and isolated complexes calculated from the protein sequence (personal communication with Michael Cosgrove).

Interestingly, analysis of BCvif₉₅₋₁₅₅ reveals that complexes from which the ¹⁶¹PPLP¹⁶⁴ motif has been removed also exhibit self-associative properties (FIG. 16C). Taken together, these data from BCvif₉₅₋₁₉₂ and BCvif₉₅₋₁₅₅, suggest that a determinant for oligomerization resides in vif outside the ¹⁶¹PPLP¹⁶⁴ motif. Dynamic light scattering from the BCvif_{SS} complex also revealed a lower Hr as compared to the wild-type BCvif complex. Based on this observation and the experimental sedimentation coefficient distribution of BCvi₉₅₋₁₅₅, it is believed that oligomerization of vif is mediated through conserved hydrophobic residues within the zinc-binding domain. To test this hypothesis, sedimentation velocity AUC was conducted on the BCvif_{SS} complex, in which two of the conserved hydrophobic residues of the HCCH motif, A123 and L124, were mutated to Ser (FIG. 12). Unlike the wild-type complex, BCvif₉₅₋₁₉₂, this mutant complex was monodisperse and the sedimentation coefficient (2.52 S*) was consistent with that of an isolated tripartite complex (FIG. 16D). Varying the concentration over a 10-fold range (0.18, 0.36, 1.8 mg ml⁻¹) did not alter the sedimentation coefficient. This observation bolsters the hypothesis that hydrophobic residues of the HCCH motif contribute to oligomerization of vif, while the PPLP motif is dispensable for oligomerization. The ability of this mutant complex to bind Cul5(N) at near wild-type affinity, suggests the global conformation and core fold of vif is unaffected by the this double mutation.

Surprisingly, however, the A123S/L234S double mutant did not have the same affect when introduced into the BCvif₉₅₋₁₅₅ complex. In fact, this mutant, dubbed BCvif_{155-SS} (FIG. 12), exhibited self-associative properties similar to that of the wild-type constructs in which oligomerization occurs.

### Example 6

### Rationale for Expression of Cul5 as an N-Terminal Truncation with the Mutations V341R, L345D

Cul5 was expressed as an N-terminal truncation (residues 2-384) in lieu of the full-length 780 residue protein for several reasons. First, expression of full-length cul5 from E. coli is hindered not only by the large size, but also by the inherent insolubility of the globular C-terminal domain of cullins, including Cul5, when expressed without its cognate binding partner, Rbx1 or Rbx2. Second, the region of Cul5 interacting with an EloBC-vif complex is hypothesized to be confined to the extreme N-terminal region. This hypothesis is drawn from examination of the Cul1-Rbx1-Skp1-Skp2 structure (pdb id 1LDK; (1)). While there is little sequence homology among cullin family members, cullin orthologues are believed to be structurally homologous based on a similar pattern of hydrophobic residues present within the N-terminal domain. Thus, the structure of Cul1-Rbx1-Skp1-Skp2 can serve as a template for modeling other Cullin-RING E3 ubiquitin ligases (CRL). Accordingly, the N-terminal structure of Cul1, comprises 3 5-helix bundle repeats, is expected to be found in Cul5 as well. The binding interface between Cul1 and substrate adaptor, Skp1, and substrate receptor, Skp2, is primarily mediated by Cul1 H2 and H5 helices respectively. While sequence conservation is generally low among cullin paralogs (Cul1, 2, 3, 4a, 4b, 5, and 7), the greatest sequence homology (among orthologs of the paralog families) exists within putative H2 and H5 helices, suggesting that like Cul1, these two putative N-terminal helices are involved in determining substrate adaptor and substrate receptor binding for each cullin paralog, including Cul5. Thus, expression of the N-terminal domain of Cul5 is expected to be sufficient for studying the interaction between Cul5 and substrate adaptor EloBC and substrate receptor, vif.

The two hydrophobic-to-hydrophilic mutations (V341R, L345D) have been introduced to the Cul5 expression vector based on experimental outcomes with expression of the N-terminal region of Cul1 (1). In the full-length structure of Cul1, the hydrophobic residues, V367 and L371, within the third 5-helix bundle of the N-terminal domain pack against the C-terminal domain (pdb 1 LDK). Without the C-terminal domain, these residues are exposed to solvent and were hypothesized to cause the observed insolubility of the Cul1 N-terminal domain when expressed in isolation. This insolubility was overcome by introducing the mutations V367R and L371D.

Based on an homology modeling algorithm, 3D JigSaw (Bates et al., 2001 Proteins Suppl 5, 39-46), it was observed that the hydrophobic residues V341 and L345 aligned with residues V367 and L371 of Cul1. Thus, the analogous mutations, V341R and L345D, were introduced to the Cul5 expression vector.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of producing soluble Vif, said method comprising contacting a cell with an isolated nucleic acid sequence encoding ElonginB, Vif, and ElonginC in a cell; expressing ElonginB, Vif, and ElonginC polypeptide in said cell; and isolating ElonginB, Vif, and ElonginC polypeptide from so expressed said cell, wherein said Vif so isolated is soluble.

2. The method of claim 1, wherein said ElonginB and Vif are expressed as a fusion polypeptide having a protease cleavage site therebetween, and said ElonginC is expressed as an additional polypeptide.

3. The method of claim 2, wherein said fusion polypeptide comprises amino acids 1-98 of said ElonginB and amino acids 102-173 of said Vif, and said additional polypeptide comprises amino acids 17-112 of said ElonginC.

4. A method of producing soluble Cullin5, said method comprising contacting a cell with an isolated nucleic acid sequence encoding Cullin5 having point mutations V341R and L345D; expressing said Cullin5 polypeptide from said cell; and isolating said Cullin5 from said cell, wherein said Cullin5 so isolated is soluble.

5. An isolated protein complex comprising Vif, ElonginB, and ElonginC, wherein said Vif is able to bind Cullin5.

6. An isolated protein complex of claim 5 further comprising Cullin5.

7. The isolated protein complex of claim 5, wherein said isolated protein complex comprises amino acids 1-98 of said ElonginB fused to a protease cleavage site which in turn is fused to amino acids 102-173 of said Vif, and said ElonginC comprises amino acids 17-112.

8. The isolated protein complex of claim 5, wherein said isolated protein complex comprises amino acids 1-104 of said ElonginB fused to a protease cleavage site which in turn is fused to amino acids 102-173 of said Vif, and said ElonginC comprises amino acids 17-112.

9. The isolated protein complex of claim 5, wherein said isolated protein complex comprises amino acids 1-118 of said ElonginB fused to a protease cleavage site which in turn is fused to amino acids 102-173 of said Vif, and said ElonginC comprises amino acids 17-112.

10. The isolated protein complex of claim 5, wherein said isolated protein complex comprises amino acids 1-118 of said ElonginB fused to a protease cleavage site which in turn is fused to amino acids 95-173 of said Vif, and said ElonginC comprises amino acids 17-112.

11. An isolated Cullin5 polypeptide, wherein said polypeptide comprises point mutations V341 and L345D.

12. A method of identifying a compound that binds to Vif, said method comprising contacting Vif with a test compound under conditions that are effective for binding of the compound with Vif; and detecting whether or not the test compound binds to Vif, wherein detection of the test compound bound to Vif identifies a compound that binds to Vif.

13. A method of identifying a compound that inhibits the interaction between Vif and Cullin5, said method comprising contacting a protein complex comprising ElonginB, ElonginC, Vif, and Cullin5 with a test compound under conditions that are effective for binding of Vif to Cullin5; and detecting whether or not the test compound disrupts binding of Vif to Cullin5, wherein when binding is disrupted, the test compound inhibits the interaction between Vif and Cullin5.

14. The method of claim 13, wherein the test compound that disrupts the binding between Vif and Cullin5 is an inhibitor of lentiviral infectivity.

15. The method of claim 13, wherein said method is a high throughput method.

16. The method of claim 15, wherein said high throughput method is Förster quenched resonance energy transfer (FqRET).

17. A compound identified by the method of claim 12.

18. A compound identified by the method of claim 13.

19. A method for inhibiting infectivity of a lentivirus, the method comprising contacting a cell which is producing the virus with an antiviral-effective amount of a compound identified by the method of claim 17, wherein the antiviral-effective amount of the compound does not substantially affect proteins in the cell other than lentivirus Vif.

20. The method of claim 19, wherein the lentivirus expresses Vif.

21. The method of claim 19, wherein the lentivirus is HIV.

22. The method of claim 19, wherein the compound inhibits the interaction of Vif with cellular Cullin5-E3 ubiquitin ligase, thereby preventing the degradation of the viral inhibitor, Apobec3G, and thus allowing the Apobec3G to inhibit viral infectivity.

23. A method for inhibiting Vif protein activity in a cell, said method comprising contacting Vif protein with an inhibitory-effective amount of a compound identified by the method of claim 11, wherein the inhibitory-effective amount of compound does not substantially affect proteins in the cell other than Vif.

24. A vector for coexpression of at least two target polynucleotides, wherein the first target polynucleotide comprises sequences encoding amino acids 1-98 of ElonginB and amino acids 102-173 of Vif having a flexible linker therebetween, and the second target polynucleotide comprises sequences encoding ElonginC, further wherein said linker comprises sequences encoding a protease cleavage site.

25. An isolated nucleic acid molecule comprising sequences encoding amino acids 1-98 of ElonginB and amino acids 102-173 of Vif having a flexible linker sequence therebetween, wherein said linker sequence comprises sequences encoding a protease cleavage site.

26. An isolated nucleic acid molecule comprising sequences encoding Cullin5, wherein said encoded Cullin5 comprises the V341 and L345D point mutations.


SEQUENCE LISTING (53 sequences)

SEQ ID NO: 1 - protein, 194 aa, Artificial
  Synthetic construct
MGDVFLMIRRHKTTIFTDAKESSTVFELKRIVEGILKRPPDEQRLYKDDQLLDDGKTLGECGFTSQTARPQAPATVGLAFRADDTFEALCIEPFSSPPELGGGGTSGGGGSGGSLADQLIHLYYFDCFSDSAIRKALLGHIVSPRCEYQAGHNKVGSLQYLALAALITPKKIKPPLPSVTKLTEDRGAHHHHHH

SEQ ID NO: 2 - protein, 97 aa, Artificial
  Synthetic construct
MGYVKLISSDGHEFIVKREHALTSGTIKAMLSGPGQFAENETNEVNFREIPSHVLSKVCMYFTYKVRYTNSSTEIPEFPIAPEIALELLMAANFLDC

SEQ ID NO: 3 - protein, 35 aa, Artificial
  Synthetic construct
ENLYFQSASGGHHHHGHHHHTSGGENLYFQSGGGS

SEQ ID NO: 4 - protein, 9 aa, Artificial
  Synthetic construct
HHHHGHHHH

SEQ ID NO: 5 - protein, 212 aa, Artificial
  Synthetic construct
MGDVFLMIRRHKTTIFTDAKESSTVFELKRIVEGILKRPPDEQRLYKDDQLLDDGKTLGECGFTSQTARPQAPATVGLAFRADDTFEALCIEPFSSPPELPDVMKENLYFQSASGGHHHHGHHHHTSGGENLYFQSGGGSLADQLIHLYYFDCFSDSAIRKALLGHIVSPRCEYQAGHNKVGSLQYLALAALITPKKIKPPLPSVTKLTEDR

SEQ ID NO: 6 - protein, 226 aa, Artificial
  Synthetic construct
MGDVFLMIRRHKTTIFTDAKESSTVFELKRIVEGILKRPPDEQRLYKDDQLLDDGKTLGECGFTSQTARPQAPATVGLAFRADDTFEALCIEPFSSPPELPDVMKPQDSGSSANEQAVQENLYFQSASGGHHHHGHHHHTSGGENLYFQSGGGSLADQLIHLYYFDCFSDSAIRKALLGHIVSPRCEYQAGHNKVGSLQYLALAALITPKKIKPPLPSVTKLTEDR

SEQ ID NO: 7 - protein, 232 aa, Artificial
  Synthetic construct
MGDVFLMIRRHKTTIFTDAKESSTVFELKRIVEGILKRPPDEQRLYKDDQLLDDGKTLGECGFTSQTARPQAPATVGLAFRADDTFEALCIEPFSSPPELPDVMKPQDSGSSANEQAVQENLYFQSASGGHHHHGHHHHTSGGENLYFQSGGGSTQVDPELADQLIHLYYFDCFSDSAIRKALLGHIVSPRCEYQAGHNKVGSLQYLALAALITPKKIKPPLPSVTKLTEDR

SEQ ID NO: 8 - protein, 617 aa, Artificial
  Synthetic construct
MGSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPMHMGGSTSNLLKNKGSLQFEDKWDFMRPIVLKLLRQESVTKQQWFDLFSDVHAVCLWDDKGPAKIHQALKEDILEFIKQAQARVLSHQDDTALLKAYIVEWRKFFTQCDILPKPFCQLEITLMGKQGSNKKSNVEDSIVRKLMLDTWNESIFSNIKNRLQDSAMKLVHAERLGEAFDSQLVIGVRESYVNLCSNPEDKLQIYRDNFEKAYLDSTERFYRTQAPSYLQQNGVQNYMKYADAKLKEEEKRALRYLETRRECNSVEALMECCVNALVTSFKETILAECQGMIKRNETEKLHLMFSLMDKVPNGIEPMLKDLEEHIISAGLADMVAAAETITTDSEKYREQLDTLFNRFSKLVKEAFQDDPRFLTARDKAYKAVVNDATIFK

SEQ ID NO: 9 - protein, 79 aa, Artificial
  Synthetic construct
STQVDPELADQLIHLYYFDCFSDSAIRKALLGHIVSPRCEYQAGHNKVGSLQYLALAALITPKKIKPPLPSVTKLTEDR

SEQ ID NO: 10 - protein, 119 aa, Artificial
  Synthetic construct
MGDVFLMIRRHKTTIFTDAKESSTVFELKRIVEGILKRPPDEQRLYKDDQLLDDGKTLGECGFTSQTARPQAPATVGLAFRADDTFEALCIEPFSSPPELPDVMKPQDSGSSANEQAVQ

SEQ ID NO: 11 - protein, 389 aa, Artificial
  Synthetic construct
PMHMGGSTSNLLKNKGSLQFEDKWDFMRPIVLKLLRQESVTKQQWFDLFSDVHAVCLWDDKGPAKIHQALKEDILEFIKQAQARVLSHQDDTALLKAYIVEWRKFFTQCDILPKPFCQLEITLMGKQGSNKKSNVEDSIVRKLMLDTWNESIFSNIKNRLQDSAMKLVHAERLGEAFDSQLVIGVRESYVNLCSNPEDKLQIYRDNFEKAYLDSTERFYRTQAPSYLQQNGVQNYMKYADAKLKEEEKRALRYLETRRECNSVEALMECCVNALVTSFKETILAECQGMIKRNETEKLHLMFSLMDKVPNGIEPMLKDLEEHIISAGLADMVAAAETITTDSEKYREQLDTLFNRFSKLVKEAFQDDPRFLTARDKAYKAVVNDATIFK

SEQ ID NO: 12 - protein, 105 aa, Artificial
  Synthetic construct
MGDVFLMIRRHKTTIFTDAKESSTVFELKRIVEGILKRPPDEQRLYKDDQLLDDGKTLGECGFTSQTARPQAPATVGLAFRADDTFEALCIEPFSSPPELPDVMK

SEQ ID NO: 13 - protein, 98 aa, Artificial
  Synthetic construct
MGDVFLMIRRHKTTIFTDAKESSTVFELKRIVEGILKRPPDEQRLYKDDQLLDDGKTLGECGFTSQTARPQAPATVGLAFRADDTFEALCIEPFSSPP

SEQ ID NO: 14 - protein, 72 aa, Artificial
  Synthetic construct
LADQLIHLYYFDCFSDSAIRKALLGHIVSPRCEYQAGHNKVGSLQYLALAALITPKKIKPPLPSVTKLTEDR

SEQ ID NO: 15 - protein, 14 aa, Artificial
  Synthetic construct
GGGGTSGGGGSGGS

SEQ ID NO: 16 - protein, 98 aa, Artificial
  Synthetic construct
STQVDPELADQLIHLYYFDCFSDSAIRKALLGHIVSPRCEYQAGHNKVGSLQYLALAALITPKKIKPPLPSVTKLTEDRWNKPQKTKGHRGSHTMNGH

SEQ ID NO: 17 - DNA, 815 bp, Artificial
  Synthetic construct
atgcgtccggcgtagaggatcgagatcgatctcgatcccgcgaaattaatacgactcactataggggaattgtgagcggataacaattcccctctagaaataattttgtttaactttaagaaggagatataccatgggcgatgtgttcctgatgatccgccgtcacaagaccacgatttttaccgacgctaaagaatcttctaccgttttcgaactgaaacgcatcgtcgaaggtattctgaaacgcccgccggacgaacagcgtctgtataaagatgatcagctgctggatgacggcaaaaccctgggcgagtgtggtttcacttctcagacggcacgtccgcaggcccctgcaaccgttggtctggcgtttcgtgccgacgatacctttgaagctctgtgcattgaaccgttctccagcccgccagagctcggcggtggtggcactagtggtggtggcggctccggcggatccctggcggatcagctgatccacctgtactacttcgactgcttctctgacagcgcgatccgtaaagcgctgctgggtcatatcgtttccccacgttgtgagtaccaggcaggccacaacaaagtgggttccctgcaatatctggctctggcggctctgatcactccgaagaagatcaaaccgccgctgccgagcgtaactaaactgaccgaagaccgtggcgcccaccaccaccatcatcactaagaattcgagctcggcgcgcctgcaggtcgacaagcttgcggccgcataatgcttaagtcgaacagaaagtaatcgtattgtacacggccgcataatc

SEQ ID NO: 18 - DNA, 570 bp, Artificial
  Synthetic construct
ttgtacacggccgcataatcgaaattaatacgactcactataggggaattgtgagcggataacaattccccatcttagtatattagttaagtataagaaggagatatacatatgggatatgtcaaattgatatcatctgatggccatgaatttattgtaaaaagagaacatgcattaacatcaggcacgataaaagccatgttgagtggcccaggtcagtttgctgagaacgaaaccaatgaggtcaattttagagagataccttcacatgtgctatcgaaagtatgcatgtattttacgtacaaggttcgctacactaacagctccaccgagattcctgaattcccaattgcacctgaaattgcactggaactgctgatggctgcgaacttcttagattgttagtagagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagc

SEQ ID NO: 19 - protein, 194 aa, Artificial; identical to SEQ ID NO: 1
  Synthetic construct

SEQ ID NO: 20 - protein, 97 aa, Artificial; identical to SEQ ID NO: 2
  Synthetic construct

SEQ ID NO: 21 - DNA, 850 bp, Artificial
  Synthetic construct
atgcgtccggcgtagaggatcgagatcgatctcgatcccgcgaaattaatacgactcactataggggaattgtgagcggataacaattcccctctagaaataattttgtttaactttaagaaggagatataccatgggcgatgtgttcctgatgatccgccgtcacaagaccacgatttttaccgacgctaaagaatcttctaccgttttcgaactgaaacgcatcgtcgaaggtattctgaaacgcccgccggacgaacagcgtctgtataaagatgatcagctgctggatgacggcaaaaccctgggcgagtgtggtttcacttctcagacggcacgtccgcaggcccctgcaaccgttggtctggcgtttcgtgccgacgatacctttgaagctctgtgcattgaaccgttctccagcccgccagagctccccgatgtgatgaaggaaaacctgtattttcagtctgctagcggaggccaccaccaccatggccaccaccaccatactagtggtggtgaaaacctgtattttcagtctggcggtggatccctggcggatcagctgatccacctgtactacttcgactgcttctctgacagcgcgatccgtaaagcgctgctgggtcatatcgtttccccacgttgtgagtaccaggcaggccacaacaaagtgggttccctgcaatatctggctctggcggctctgatcactccgaagaagatcaaaccgccgctgccgagcgtaactaaactgaccgaagaccgttaactgcaggtcgacaagcttgcggccgcataatgcttaagtcgaacagaaagtaatcgtattgtacacggccgcataatc

SEQ ID NO: 22 - protein, 212 aa, Artificial; identical to SEQ ID NO: 5
  Synthetic construct

SEQ ID NO: 23 - protein, 111 aa, Artificial
  Synthetic construct
MGDVFLMIRRHKTTIFTDAKESSTVFELKRIVEGILKRPPDEQRLYKDDQLLDDGKTLGECGFTSQTARPQAPATVGLAFRADDTFEALCIEPFSSPPELPDVMKENLYFQ

SEQ ID NO: 24 - protein, 77 aa, Artificial
  Synthetic construct
SGGGSLADQLIHLYYFDCFSDSAIRKALLGHIVSPRCEYQAGHNKVGSLQYLALAALITPKKIKPPLPSVTKLTEDR

SEQ ID NO: 25 - protein, 24 aa, Artificial
  Synthetic construct
SASGGHHHHGHHHHTSGGENLYFQ

SEQ ID NO: 26 - DNA, 910 bp, Artificial
  Synthetic construct
atgcgtccggcgtagaggatcgagatcgatctcgatcccgcgaaattaatacgactcactataggggaattgtgagcggataacaattcccctctagaaataattttgtttaactttaagaaggagatataccatgggcgatgtgttcctgatgatccgccgtcacaagaccacgatttttaccgacgctaaagaatcttctaccgttttcgaactgaaacgcatcgtcgaaggtattctgaaacgcccgccggacgaacagcgtctgtataaagatgatcagctgctggatgacggcaaaaccctgggcgagtgtggtttcacttctcagacggcacgtccgcaggcccctgcaaccgttggtctggcgtttcgtgccgacgatacctttgaagctctgtgcattgaaccgttctccagcccgccagagctccccgatgtgatgaagccgcaggactctggttcttctgcgaacgaacaggcggttcaggaaaacctgtattttcagtctgctagcggaggccaccaccaccatggccaccaccaccatactagtggtggtgaaaacctgtattttcagtctggcggtggatccacacaagtagaccctgaactagcagaccaactaattcatctgtattactttgactgtttttcagactctgctataagaaaggccttattaggacatatagttagccctaggtgtgaatatcaagcaggacataacaaggtaggatctctacaatacttggcactagcagcattaataacaccaaaaaagataaagccacctttgcctagtgttacgaaactgacagaggatagataactgcaggtcgacaagcttgcggccgcataatgcttaagtcgaacagaaagtaatcgtattgtacacggccgcataatc

SEQ ID NO: 27 - protein, 218 aa, Artificial
  Synthetic construct
MGDVFLMIRRHKTTIFTDAKESSTVFELKRIVEGILKRPPDEQRLYKDDQLLDDGKTLGECGFTSQTARPQAPATVGLAFRADDTFEALCIEPFSSPPELPDVMKENLYFQSASGGHHHHGHHHHTSGGENLYFQSGGGSTQVDPELADQLIHLYYFDCFSDSAIRKALLGHIVSPRCEYQAGHNKVGSLQYLALAALITPKKIKPPLPSVTKLTEDR

SEQ ID NO: 28 - protein, 83 aa, Artificial
  Synthetic construct
SGGGSTQVDPELADQLIHLYYFDCFSDSAIRKALLGHIVSPRCEYQAGHNKVGSLQYLALAALITPKKIKPPLPSVTKLTEDR

SEQ ID NO: 29 - DNA, 967 bp, Artificial
  Synthetic construct
atgcgtccggcgtagaggatcgagatcgatctcgatcccgcgaaattaatacgactcactataggggaattgtgagcggataacaattcccctctagaaataattttgtttaactttaagaaggagatataccatgggcgatgtgttcctgatgatccgccgtcacaagaccacgatttttaccgacgctaaagaatcttctaccgttttcgaactgaaacgcatcgtcgaaggtattctgaaacgcccgccggacgaacagcgtctgtataaagatgatcagctgctggatgacggcaaaaccctgggcgagtgtggtttcacttctcagacggcacgtccgcaggcccctgcaaccgttggtctggcgtttcgtgccgacgatacctttgaagctctgtgcattgaaccgttctccagcccgccagagctccccgatgtgatgaagccgcaggactctggttcttctgcgaacgaacaggcggttcaggaaaacctgtattttcagtctgctagcggaggccaccaccaccatggccaccaccaccatactagtggtggtgaaaacctgtattttcagtctggcggtggatccacacaagtagaccctgaactagcagaccaactaattcatctgtattactttgactgtttttcagactctgctataagaaaggccttattaggacatatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactaactgcaggtcgacaagcttgcggccgcataatgcttaagtcgaacagaaagtaatcgtattgtacacggccgcataatc

SEQ ID NO: 30 - protein, 237 aa, Artificial
  Synthetic construct
MGDVFLMIRRHKTTIFTDAKESSTVFELKRIVEGILKRPPDEQRLYKDDQLLDDGKTLGECGFTSQTARPQAPATVGLAFRADDTFEALCIEPFSSPPELPDVMKENLYFQSASGGHHHHGHHHHTSGGENLYFQSGGGSTQVDPELADQLIHLYYFDCFSDSAIRKALLGHIVSPRCEYQAGHNKVGSLQYLALAALITPKKIKPPLPSVTKLTEDRWNKPQKTKGHRGSHTMNGH

SEQ ID NO: 31 - protein, 111 aa, Artificial; identical to SEQ ID NO: 23
  Synthetic construct

SEQ ID NO: 32 - protein, 102 aa, Artificial
  Synthetic construct
SGGGSTQVDPELADQLIHLYYFDCFSDSAIRKALLGHIVSPRCEYQAGHNKVGSLQYLALAALITPKKIKPPLPSVTKLTEDRWNKPQKTKGHRGSHTMNGH

SEQ ID NO: 33 - DNA, 910 bp, Artificial; identical to SEQ ID NO: 26
  Synthetic construct

SEQ ID NO: 34 - protein, 218 aa, Artificial; identical to SEQ ID NO: 27
  Synthetic construct

SEQ ID NO: 35 - DNA, 871 bp, Artificial
  Synthetic construct
atgcgtccggcgtagaggatcgagatcgatctcgatcccgcgaaattaatacgactcactataggggaattgtgagcggataacaattcccctctagaaataattttgtttaactttaagaaggagatataccatgggcgatgtgttcctgatgatccgccgtcacaagaccacgatttttaccgacgctaaagaatcttctaccgttttcgaactgaaacgcatcgtcgaaggtattctgaaacgcccgccggacgaacagcgtctgtataaagatgatcagctgctggatgacggcaaaaccctgggcgagtgtggtttcacttctcagacggcacgtccgcaggcccctgcaaccgttggtctggcgtttcgtgccgacgatacctttgaagctctgtgcattgaaccgttctccagcccgccagagctccccgatgtgatgaagccgcaggactctggttcttctgcgaacgaacaggcggttcaggaaaacctgtattttcagtctgctagcggaggccaccaccaccatggccaccaccaccatactagtggtggtgaaaacctgtattttcagtctggcggtggatccacacaagtagaccctgaactagcagaccaactaattcatctgtattactttgactgtttttcagactctgctataagaaaggccttattaggacatatagttagccctaggtgtgaatatcaagcaggacataacaaggtaggatctctacaatacttggcactagcagcattaataacaccaaaaaagataaagtaactgcaggtcgacaagcttgcggccgcataatgcttaagtcgaacagaaagtaatcgtattgtacacggccgcataatc

SEQ ID NO: 36 - protein, 205 aa, Artificial
  Synthetic construct
MGDVFLMIRRHKTTIFTDAKESSTVFELKRIVEGILKRPPDEQRLYKDDQLLDDGKTLGECGFTSQTARPQAPATVGLAFRADDTFEALCIEPFSSPPELPDVMKENLYFQSASGGHHHHGHHHHTSGGENLYFQSGGGSTQVDPELADQLIHLYYFDCFSDSAIRKALLGHIVSPRCEYQAGHNKVGSLQYLALAALITPKKIK

SEQ ID NO: 37 - protein, 70 aa, Artificial
  Synthetic construct
SGGGSTQVDPELADQLIHLYYFDCFSDSAIRKALLGHIVSPRCEYQAGHNKVGSLQYLALAALITPKKIK

SEQ ID NO: 38 - DNA, 967 bp, Artificial
  Synthetic construct
atgcgtccggcgtagaggatcgagatcgatctcgatcccgcgaaattaatacgactcactataggggaattgtgagcggataacaattcccctctagaaataattttgtttaactttaagaaggagatataccatgggcgatgtgttcctgatgatccgccgtcacaagaccacgatttttaccgacgctaaagaatcttctaccgttttcgaactgaaacgcatcgtcgaaggtattctgaaacgcccgccggacgaacagcgtctgtataaagatgatcagctgctggatgacggcaaaaccctgggcgagtgtggtttcacttctcagacggcacgtccgcaggcccctgcaaccgttggtctggcgtttcgtgccgacgatacctttgaagctctgtgcattgaaccgttctccagcccgccagagctccccgatgtgatgaagccgcaggactctggttcttctgcgaacgaacaggcggttcaggaaaacctgtattttcagtctgctagcggaggccaccaccaccatggccaccaccaccatactagtggtggtgaaaacctgtattttcagtctggcggtggatccacacaagtagaccctgaactagcagaccaactaattcatctgtattactttgactgtttttcagactctgctataagaaaggccttattaggacatatagttagccctaggtgtgaatatcaagcaggacataacaaggtaggatctctacaatacttggcactagcagcattaataacataaaaaaagataaagccacctttgcctagtgttacgaaactgacagaggatagatggaacaagccccagaagaccaagggccacagagggagccacacaatgaatggacactaactgcaggtcgacaagcttgcggccgcataatgcttaagtcgaacagaaagtaatcgtattgtacacggccgcataatc

SEQ ID NO: 39 - protein, 200 aa, Artificial
  Synthetic construct
MGDVFLMIRRHKTTIFTDAKESSTVFELKRIVEGILKRPPDEQRLYKDDQLLDDGKTLGECGFTSQTARPQAPATVGLAFRADDTFEALCIEPFSSPPELPDVMKENLYFQSASGGHHHHGHHHHTSGGENLYFQSGGGSTQVDPELADQLIHLYYFDCFSDSAIRKALLGHIVSPRCEYQAGHNKVGSLQYLALAALIT

SEQ ID NO: 40 - protein, 65 aa, Artificial
  Synthetic construct
SGGGSTQVDPELADQLIHLYYFDCFSDSAIRKALLGHIVSPRCEYQAGHNKVGSLQYLALAALIT

SEQ ID NO: 41 - DNA, 967 bp, Artificial
  Synthetic construct
atgcgtccggcgtagaggatcgagatcgatctcgatcccgcgaaattaatacgactcactataggggaattgtgagcggataacaattcccctctagaaataattttgtttaactttaagaaggagatataccatgggcgatgtgttcctgatgatccgccgtcacaagaccacgatttttaccgacgctaaagaatcttctaccgttttcgaactgaaacgcatcgtcgaaggtattctgaaacgcccgccggacgaacagcgtctgtataaagatgatcagctgctggatgacggcaaaaccctgggcgagtgtggtttcacttctcagacggcacgtccgcaggcccctgcaaccgttggtctggcgtttcgtgccgacgatacctttgaagctctgtgcattgaaccgttctccagcccgccagagctccccgatgtgatgaagccgcaggactctggttcttctgcgaacgaacaggcggttcaggaaaacctgtattttcagtctgctagcggaggccaccaccaccatggccaccaccaccatactagtggtggtgaaaacctgtattttcagtctggcggtggatccacacaagtagaccctgaactagcagaccaactaattcatctgtattactttgactgtttttcagactctgctataagaaagggcgcattaggacatatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactaactgcaggtcgacaagcttgcggccgcataatgcttaagtcgaacagaaagtaatcgtattgtacacggccgcataatc

SEQ ID NO: 42 - protein, 237 aa, Artificial
  Synthetic construct
MGDVFLMIRRHKTTIFTDAKESSTVFELKRIVEGILKRPPDEQRLYKDDQLLDDGKTLGECGFTSQTARPQAPATVGLAFRADDTFEALCIEPFSSPPELPDVMKENLYFQSASGGHHHHGHHHHTSGGENLYFQSGGGSTQVDPELADQLIHLYYFDCFSDSAIRKGALGHIVSPRCEYQAGHNKVGSLQYLALAALITPKKIKPPLPSVTKLTEDRWNKPQKTKGHRGSHTMNGH

SEQ ID NO: 43 - protein, 102 aa, Artificial
  Synthetic construct
SGGGSTQVDPELADQLIHLYYFDCFSDSAIRKGALGHIVSPRCEYQAGHNKVGSLQYLALAALITPKKIKPPLPSVTKLTEDRWNKPQKTKGHRGSHTMNGH

SEQ ID NO: 44 - DNA, 967 bp, Artificial
  Synthetic construct
atgcgtccggcgtagaggatcgagatcgatctcgatcccgcgaaattaatacgactcactataggggaattgtgagcggataacaattcccctctagaaataattttgtttaactttaagaaggagatataccatgggcgatgtgttcctgatgatccgccgtcacaagaccacgatttttaccgacgctaaagaatcttctaccgttttcgaactgaaacgcatcgtcgaaggtattctgaaacgcccgccggacgaacagcgtctgtataaagatgatcagctgctggatgacggcaaaaccctgggcgagtgtggtttcacttctcagacggcacgtccgcaggcccctgcaaccgttggtctggcgtttcgtgccgacgatacctttgaagctctgtgcattgaaccgttctccagcccgccagagctccccgatgtgatgaagccgcaggactctggttcttctgcgaacgaacaggcggttcaggaaaacctgtattttcagtctgctagcggaggccaccaccaccatggccaccaccaccatactagtggtggtgaaaacctgtattttcagtctggcggtggatccacacaagtagaccctgaactagcagaccaactaattcatctgtattactttgactgtttttcagactctgctataagaaaggtcttcttaggacatatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactaactgcaggtcgacaagcttgcggccgcataatgcttaagtcgaacagaaagtaatcgtattgtacacggccgcataatc

SEQ ID NO: 45 - protein, 237 aa, Artificial
  Synthetic construct
MGDVFLMIRRHKTTIFTDAKESSTVFELKRIVEGILKRPPDEQRLYKDDQLLDDGKTLGECGFTSQTARPQAPATVGLAFRADDTFEALCIEPFSSPPELPDVMKENLYFQSASGGHHHHGHHHHTSGGENLYFQSGGGSTQVDPELADQLIHLYYFDCFSDSAIRKVFLGHIVSPRCEYQAGHNKVGSLQYLALAALITPKKIKPPLPSVTKLTEDRWNKPQKTKGHRGSHTMNGH

SEQ ID NO: 46 - protein, 102 aa, Artificial
  Synthetic construct
SGGGSTQVDPELADQLIHLYYFDCFSDSAIRKVFLGHIVSPRCEYQAGHNKVGSLQYLALAALITPKKIKPPLPSVTKLTEDRWNKPQKTKGHRGSHTMNGH

SEQ ID NO: 47 - DNA, 967 bp, Artificial
  Synthetic construct
atgcgtccggcgtagaggatcgagatcgatctcgatcccgcgaaattaatacgactcactataggggaattgtgagcggataacaattcccctctagaaataattttgtttaactttaagaaggagatataccatgggcgatgtgttcctgatgatccgccgtcacaagaccacgatttttaccgacgctaaagaatcttctaccgttttcgaactgaaacgcatcgtcgaaggtattctgaaacgcccgccggacgaacagcgtctgtataaagatgatcagctgctggatgacggcaaaaccctgggcgagtgtggtttcacttctcagacggcacgtccgcaggcccctgcaaccgttggtctggcgtttcgtgccgacgatacctttgaagctctgtgcattgaaccgttctccagcccgccagagctccccgatgtgatgaagccgcaggactctggttcttctgcgaacgaacaggcggttcaggaaaacctgtattttcagtctgctagcggaggccaccaccaccatggccaccaccaccatactagtggtggtgaaaacctgtattttcagtctggcggtggatccacacaagtagaccctgaactagcagaccaactaattcatctgtattactttgactgtttttcagactctgctataagaaagtcctcattaggacatatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactaactgcaggtcgacaagcttgcggccgcataatgcttaagtcgaacagaaagtaatcgtattgtacacggccgcataatc

SEQ ID NO: 48 - protein, 237 aa, Artificial
  Synthetic construct
MGDVFLMIRRHKTTIFTDAKESSTVFELKRIVEGILKRPPDEQRLYKDDQLLDDGKTLGECGFTSQTARPQAPATVGLAFRADDTFEALCIEPFSSPPELPDVMKENLYFQSASGGHHHHGHHHHTSGGENLYFQSGGGSTQVDPELADQLIHLYYFDCFSDSAIRKSSLGHIVSPRCEYQAGHNKVGSLQYLALAALITPKKIKPPLPSVTKLTEDRWNKPQKTKGHRGSHTMNGH

SEQ ID NO: 49 - protein, 102 aa, Artificial
  Synthetic construct
SGGGSTQVDPELADQLIHLYYFDCFSDSAIRKSSLGHIVSPRCEYQAGHNKVGSLQYLALAALITPKKIKPPLPSVTKLTEDRWNKPQKTKGHRGSHTMNGH

SEQ ID NO: 50 - DNA, 967 bp, Artificial
  Synthetic construct
atgcgtccggcgtagaggatcgagatcgatctcgatcccgcgaaattaatacgactcactataggggaattgtgagcggataacaattcccctctagaaataattttgtttaactttaagaaggagatataccatgggcgatgtgttcctgatgatccgccgtcacaagaccacgatttttaccgacgctaaagaatcttctaccgttttcgaactgaaacgcatcgtcgaaggtattctgaaacgcccgccggacgaacagcgtctgtataaagatgatcagctgctggatgacggcaaaaccctgggcgagtgtggtttcacttctcagacggcacgtccgcaggcccctgcaaccgttggtctggcgtttcgtgccgacgatacctttgaagctctgtgcattgaaccgttctccagcccgccagagctccccgatgtgatgaagccgcaggactctggttcttctgcgaacgaacaggcggttcaggaaaacctgtattttcagtctgctagcggaggccaccaccaccatggccaccaccaccatactagtggtggtgaaaacctgtattttcagtctggcggtggatccacacaagtagaccctgaactagcagaccaactaattcatctgtattactttgactgtttttcagactctgctataagaaagtcctcattaggacatatagttagccctaggtgtgaatatcaagcaggacataacaaggtaggatctctacaatacttggcactagcagcattaataacataaaaaaagataaagccacctttgcctagtgttacgaaactgacagaggatagatggaacaagccccagaagaccaagggccacagagggagccacacaatgaatggacactaactgcaggtcgacaagcttgcggccgcataatgcttaagtcgaacagaaagtaatcgtattgtacacggccgcataatc

SEQ ID NO: 51 - DNA, 910 bp, Artificial
  Synthetic construct
atgcgtccggcgtagaggatcgagatcgatctcgatcccgcgaaattaatacgactcactataggggaattgtgagcggataacaattcccctctagaaataattttgtttaactttaagaaggagatataccatgggcgatgtgttcctgatgatccgccgtcacaagaccacgatttttaccgacgctaaagaatcttctaccgttttcgaactgaaacgcatcgtcgaaggtattctgaaacgcccgccggacgaacagcgtctgtataaagatgatcagctgctggatgacggcaaaaccctgggcgagtgtggtttcacttctcagacggcacgtccgcaggcccctgcaaccgttggtctggcgtttcgtgccgacgatacctttgaagctctgtgcattgaaccgttctccagcccgccagagctccccgatgtgatgaagccgcaggactctggttcttctgcgaacgaacaggcggttcaggaaaacctgtattttcagtctgctagcggaggccaccaccaccatggccaccaccaccatactagtggtggttaaaacctgtattttcagtctggcggtggatccacacaagtagaccctgaactagcagaccaactaattcatctgtattactttgactgtttttcagactctgctataagaaaggccttattaggacatatagttagccctaggtgtgaatatcaagcaggacataacaaggtaggatctctacaatacttggcactagcagcattaataacaccaaaaaagataaagccacctttgcctagtgttacgaaactgacagaggatagataactgcaggtcgacaagcttgcggccgcataatgcttaagtcgaacagaaagtaatcgtattgtacacggccgcataatc

SEQ ID NO: 52 - protein, 129 aa, Artificial
  Synthetic construct
MGDVFLMIRRHKTTIFTDAKESSTVFELKRIVEGILKRPPDEQRLYKDDQLLDDGKTLGECGFTSQTARPQAPATVGLAFRADDTFEALCIEPFSSPPELPDVMKENLYFQSASGGHHHHGHHHHTSGG

SEQ ID NO: 53 - protein, 18 aa, Artificial
  Synthetic construct
SASGGHHHHGHHHHTSGG